SEQUENCE LISTING (173 sequences)

SEQ ID NO: 1 - RNA, 37 nt, synthetic construct
  source 1..37: mol_type = other RNA
cgtcattgagcagtactcgcagggccacctcaacctc

SEQ ID NO: 2 - RNA, 37 nt, synthetic construct
  source 1..37: mol_type = other RNA
cgtcattgagcagtactcacagggccacctcaacctc

SEQ ID NO: 3 - RNA, 37 nt, synthetic construct
  source 1..37: mol_type = other RNA
gaggttgaggtggccctgcgagtactgctcaatgacg

SEQ ID NO: 4 - RNA, 37 nt, synthetic construct
  source 1..37: mol_type = other RNA
gaggttgaggtggccctgtgagtactgctcaatgacg

SEQ ID NO: 5 - DNA, 59 bp, synthetic construct
  source 1..59: mol_type = other DNA
ccggaacacagggccacctcaaccttcaagacaggttgaggtggccctgtgtttttttg

SEQ ID NO: 6 - DNA, 59 bp, synthetic construct
  source 1..59: mol_type = other DNA
aattcaaaaaaacacagggccacctcaacctgtcttgaaggttgaggtggccctgtgtt

SEQ ID NO: 7 - DNA, 59 bp, synthetic construct
  source 1..59: mol_type = other DNA
ccggaagcagtactcacagggccactcaagacgtggccctgtgagtactgctttttttg

SEQ ID NO: 8 - DNA, 59 bp, synthetic construct
  source 1..59: mol_type = other DNA
aattcaaaaaaagcagtactcacagggccacgtcttgagtggccctgtgagtactgctt

SEQ ID NO: 9 - DNA, 59 bp, synthetic construct
  source 1..59: mol_type = other DNA
ccggaagtactcacagggccacctctcaagacgaggtggccctgtgagtactttttttg

SEQ ID NO: 10 - DNA, 59 bp, synthetic construct
  source 1..59: mol_type = other DNA
aattcaaaaaaagtactcacagggccacctcgtcttgagaggtggccctgtgagtactt

SEQ ID NO: 11 - DNA, 59 bp, synthetic construct
  source 1..59: mol_type = other DNA
ccggaaactcacagggccacctcaatcaagacttgaggtggccctgtgagttttttttg

SEQ ID NO: 12 - DNA, 59 bp, synthetic construct
  source 1..59: mol_type = other DNA
aattcaaaaaaaactcacagggccacctcaagtcttgattgaggtggccctgtgagttt

SEQ ID NO: 13 - DNA, 59 bp, synthetic construct
  source 1..59: mol_type = other DNA
ccggaactcacagggccacctcaactcaagacgttgaggtggccctgtgagtttttttg

SEQ ID NO: 14 - DNA, 59 bp, synthetic construct
  source 1..59: mol_type = other DNA
aattcaaaaaaactcacagggccacctcaacgtcttgagttgaggtggccctgtgagtt

SEQ ID NO: 15 - DNA, 59 bp, synthetic construct
  source 1..59: mol_type = other DNA
ccggaacgcagggccacctcaaccttcaagacaggttgaggtggccctgcgtttttttg

SEQ ID NO: 16 - DNA, 59 bp, synthetic construct
  source 1..59: mol_type = other DNA
aattcaaaaaaacgcagggccacctcaacctgtcttgaaggttgaggtggccctgcgtt

SEQ ID NO: 17 - DNA, 59 bp, synthetic construct
  source 1..59: mol_type = other DNA
ccggaagcagtactcgcagggccactcaagacgtggccctgcgagtactgctttttttg

SEQ ID NO: 18 - DNA, 59 bp, synthetic construct
  source 1..59: mol_type = other DNA
aattcaaaaaaagcagtactcgcagggccacgtcttgagtggccctgcgagtactgctt

SEQ ID NO: 19 - DNA, 59 bp, synthetic construct
  source 1..59: mol_type = other DNA
ccggaacagtactcgcagggccacctcaagacggtggccctgcgagtactgtttttttg

SEQ ID NO: 20 - DNA, 59 bp, synthetic construct
  source 1..59: mol_type = other DNA
aattcaaaaaaacagtactcgcagggccaccgtcttgaggtggccctgcgagtactgtt

SEQ ID NO: 21 - DNA, 59 bp, synthetic construct
  source 1..59: mol_type = other DNA
ccggaaagtactcgcagggccaccttcaagacaggtggccctgcgagtacttttttttg

SEQ ID NO: 22 - DNA, 59 bp, synthetic construct
  source 1..59: mol_type = other DNA
aattcaaaaaaaagtactcgcagggccacctgtcttgaaggtggccctgcgagtacttt

SEQ ID NO: 23 - DNA, 59 bp, synthetic construct
  source 1..59: mol_type = other DNA
ccggaactcgcagggccacctcaactcaagacgttgaggtggccctgcgagtttttttg

SEQ ID NO: 24 - DNA, 59 bp, synthetic construct
  source 1..59: mol_type = other DNA
aattcaaaaaaactcgcagggccacctcaacgtcttgagttgaggtggccctgcgagtt

SEQ ID NO: 25 - DNA, 59 bp, synthetic construct
  source 1..59: mol_type = other DNA
ccggaacagtactcgcaggggcacctcaagacggtgcccctgcgagtactgtttttttg

SEQ ID NO: 26 - DNA, 59 bp, synthetic construct
  source 1..59: mol_type = other DNA
aattcaaaaaaacagtactcgcaggggcaccgtcttgaggtgcccctgcgagtactgtt

SEQ ID NO: 27 - DNA, 59 bp, synthetic construct
  source 1..59: mol_type = other DNA
ccggaacagtactcgcaggcccacctcaagacggtgggcctgcgagtactgtttttttg

SEQ ID NO: 28 - DNA, 59 bp, synthetic construct
  source 1..59: mol_type = other DNA
aattcaaaaaaacagtactcgcaggcccaccgtcttgaggtgggcctgcgagtactgtt

SEQ ID NO: 29 - DNA, 59 bp, synthetic construct
  source 1..59: mol_type = other DNA
ccggaacagtactcgcagcgccacctcaagacggtggcgctgcgagtactgtttttttg

SEQ ID NO: 30 - DNA, 59 bp, synthetic construct
  source 1..59: mol_type = other DNA
aattcaaaaaaacagtactcgcagcgccaccgtcttgaggtggcgctgcgagtactgtt

SEQ ID NO: 31 - DNA, 59 bp, synthetic construct
  source 1..59: mol_type = other DNA
ccggaacagtactcgcagggccatatcaagacggtggccctgcgagtactgtttttttg

SEQ ID NO: 32 - DNA, 59 bp, synthetic construct
  source 1..59: mol_type = other DNA
aattcaaaaaaacagtactcgcagggccaccgtcttgatatggccctgcgagtactgtt

SEQ ID NO: 33 - DNA, 59 bp, synthetic construct
  source 1..59: mol_type = other DNA
ccggaaagtactcgcagggcgaccttcaagacaggtcgccctgcgagtacttttttttg

SEQ ID NO: 34 - DNA, 59 bp, synthetic construct
  source 1..59: mol_type = other DNA
aattcaaaaaaaagtactcgcagggcgacctgtcttgaaggtcgccctgcgagtacttt

SEQ ID NO: 35 - DNA, 59 bp, synthetic construct
  source 1..59: mol_type = other DNA
ccggaaagtactcgcaggggcaccttcaagacaggtgcccctgcgagtacttttttttg

SEQ ID NO: 36 - DNA, 59 bp, synthetic construct
  source 1..59: mol_type = other DNA
aattcaaaaaaaagtactcgcaggggcacctgtcttgaaggtgcccctgcgagtacttt

SEQ ID NO: 37 - DNA, 59 bp, synthetic construct
  source 1..59: mol_type = other DNA
ccggaaagtactcgcaggcccaccttcaagacaggtgggcctgcgagtacttttttttg

SEQ ID NO: 38 - DNA, 59 bp, synthetic construct
  source 1..59: mol_type = other DNA
aattcaaaaaaaagtactcgcaggcccacctgtcttgaaggtgggcctgcgagtacttt

SEQ ID NO: 39 - DNA, 59 bp, synthetic construct
  source 1..59: mol_type = other DNA
ccggaaagtactcgcagggccactatcaagacaggtggccctgcgagtacttttttttg

SEQ ID NO: 40 - DNA, 59 bp, synthetic construct
  source 1..59: mol_type = other DNA
aattcaaaaaaaagtactcgcagggccacctgtcttgatagtggccctgcgagtacttt

SEQ ID NO: 41 - DNA, 59 bp, synthetic construct
  source 1..59: mol_type = other DNA
ccggaacgcagggcctcctcaaccttcaagacaggttgaggaggccctgcgtttttttg

SEQ ID NO: 42 - DNA, 59 bp, synthetic construct
  source 1..59: mol_type = other DNA
aattcaaaaaaacgcagggcctcctcaacctgtcttgaaggttgaggaggccctgcgtt

SEQ ID NO: 43 - DNA, 59 bp, synthetic construct
  source 1..59: mol_type = other DNA
ccggaacgcagggcgacctcaaccttcaagacaggttgaggtcgccctgcgtttttttg

SEQ ID NO: 44 - DNA, 59 bp, synthetic construct
  source 1..59: mol_type = other DNA
aattcaaaaaaacgcagggcgacctcaacctgtcttgaaggttgaggtcgccctgcgtt

SEQ ID NO: 45 - DNA, 59 bp, synthetic construct
  source 1..59: mol_type = other DNA
ccggaacgcagggccacctcaactatcaagacaggttgaggtggccctgcgtttttttg

SEQ ID NO: 46 - DNA, 59 bp, synthetic construct
  source 1..59: mol_type = other DNA
aattcaaaaaaacgcagggccacctcaacctgtcttgatagttgaggtggccctgcgtt

SEQ ID NO: 47 - DNA, 59 bp, synthetic construct
  source 1..59: mol_type = other DNA
ccggaacacagggcgacctcaaccttcaagacaggttgaggtcgccctgtgtttttttg

SEQ ID NO: 48 - DNA, 59 bp, synthetic construct
  source 1..59: mol_type = other DNA
aattcaaaaaaacacagggcgacctcaacctgtcttgaaggttgaggtcgccctgtgtt

SEQ ID NO: 49 - DNA, 59 bp, synthetic construct
  source 1..59: mol_type = other DNA
ccggaacacagggcctcctcaaccttcaagacaggttgaggaggccctgtgtttttttg

SEQ ID NO: 50 - DNA, 59 bp, synthetic construct
  source 1..59: mol_type = other DNA
aattcaaaaaaacacagggcctcctcaacctgtcttgaaggttgaggaggccctgtgtt

SEQ ID NO: 51 - DNA, 59 bp, synthetic construct
  source 1..59: mol_type = other DNA
ccggaacacagggccacctcaactatcaagacaggttgaggtggccctgtgtttttttg

SEQ ID NO: 52 - DNA, 59 bp, synthetic construct
  source 1..59: mol_type = other DNA
aattcaaaaaaacacagggccacctcaacctgtcttgatagttgaggtggccctgtgtt

SEQ ID NO: 53 - DNA, 59 bp, synthetic construct
  source 1..59: mol_type = other DNA
ccggaactgggcattacatcgcatatcaagactatgcgatgtaatgcccagtttttttg

SEQ ID NO: 54 - DNA, 59 bp, synthetic construct
  source 1..59: mol_type = other DNA
aattcaaaaaaactgggcattacatcgcatagtcttgatatgcgatgtaatgcccagtt

SEQ ID NO: 55 - DNA, 59 bp, synthetic construct
  source 1..59: mol_type = other DNA
ccggaagggcattacatcgcatagatcaagactctatgcgatgtaatgccctttttttg

SEQ ID NO: 56 - DNA, 59 bp, synthetic construct
  source 1..59: mol_type = other DNA
aattcaaaaaaagggcattacatcgcatagagtcttgatctatgcgatgtaatgccctt

SEQ ID NO: 57 - DNA, 59 bp, synthetic construct
  source 1..59: mol_type = other DNA
ccggaaggcattacatcgcatagaatcaagacttctatgcgatgtaatgcctttttttg

SEQ ID NO: 58 - DNA, 59 bp, synthetic construct
  source 1..59: mol_type = other DNA
aattcaaaaaaaggcattacatcgcatagaagtcttgattctatgcgatgtaatgcctt

SEQ ID NO: 59 - DNA, 59 bp, synthetic construct
  source 1..59: mol_type = other DNA
ccggaagcattacatcgcatagaaatcaagactttctatgcgatgtaatgctttttttg

SEQ ID NO: 60 - DNA, 59 bp, synthetic construct
  source 1..59: mol_type = other DNA
aattcaaaaaaagcattacatcgcatagaaagtcttgatttctatgcgatgtaatgctt

SEQ ID NO: 61 - DNA, 59 bp, synthetic construct
  source 1..59: mol_type = other DNA
ccggaacattacatcgcatagaaattcaagacatttctatgcgatgtaatgtttttttg

SEQ ID NO: 62 - DNA, 59 bp, synthetic construct
  source 1..59: mol_type = other DNA
aattcaaaaaaacattacatcgcatagaaatgtcttgaatttctatgcgatgtaatgtt

SEQ ID NO: 63 - DNA, 59 bp, synthetic construct
  source 1..59: mol_type = other DNA
ccggaattacatcgcatagaaatcatcaagactgatttctatgcgatgtaatttttttg

SEQ ID NO: 64 - DNA, 59 bp, synthetic construct
  source 1..59: mol_type = other DNA
aattcaaaaaaattacatcgcatagaaatcagtcttgatgatttctatgcgatgtaatt

SEQ ID NO: 65 - DNA, 59 bp, synthetic construct
  source 1..59: mol_type = other DNA
ccggaatacatcgcatagaaatcaatcaagacttgatttctatgcgatgtatttttttg

SEQ ID NO: 66 - DNA, 59 bp, synthetic construct
  source 1..59: mol_type = other DNA
aattcaaaaaaatacatcgcatagaaatcaagtcttgattgatttctatgcgatgtatt

SEQ ID NO: 67 - DNA, 59 bp, synthetic construct
  source 1..59: mol_type = other DNA
ccggaacatcgcatagaaatcaatatcaagactattgatttctatgcgatgtttttttg

SEQ ID NO: 68 - DNA, 59 bp, synthetic construct
  source 1..59: mol_type = other DNA
aattcaaaaaaacatcgcatagaaatcaatagtcttgatattgatttctatgcgatgtt

SEQ ID NO: 69 - DNA, 59 bp, synthetic construct
  source 1..59: mol_type = other DNA
ccggaactgggcattacgtcgcatatcaagactatgcgacgtaatgcccagtttttttg

SEQ ID NO: 70 - DNA, 59 bp, synthetic construct
  source 1..59: mol_type = other DNA
aattcaaaaaaactgggcattacgtcgcatagtcttgatatgcgacgtaatgcccagtt

SEQ ID NO: 71 - DNA, 59 bp, synthetic construct
  source 1..59: mol_type = other DNA
ccggaagggcattacgtcgcatagatcaagactctatgcgacgtaatgccctttttttg

SEQ ID NO: 72 - DNA, 59 bp, synthetic construct
  source 1..59: mol_type = other DNA
aattcaaaaaaagggcattacgtcgcatagagtcttgatctatgcgacgtaatgccctt

SEQ ID NO: 73 - DNA, 59 bp, synthetic construct
  source 1..59: mol_type = other DNA
ccggaaggcattacgtcgcatagaatcaagacttctatgcgacgtaatgcctttttttg

SEQ ID NO: 74 - DNA, 59 bp, synthetic construct
  source 1..59: mol_type = other DNA
aattcaaaaaaaggcattacgtcgcatagaagtcttgattctatgcgacgtaatgcctt

SEQ ID NO: 75 - DNA, 59 bp, synthetic construct
  source 1..59: mol_type = other DNA
ccggaagcattacgtcgcatagaaatcaagactttctatgcgacgtaatgctttttttg

SEQ ID NO: 76 - DNA, 59 bp, synthetic construct
  source 1..59: mol_type = other DNA
aattcaaaaaaagcattacgtcgcatagaaagtcttgatttctatgcgacgtaatgctt

SEQ ID NO: 77 - DNA, 59 bp, synthetic construct
  source 1..59: mol_type = other DNA
ccggaacattacgtcgcatagaaattcaagacatttctatgcgacgtaatgtttttttg

SEQ ID NO: 78 - DNA, 59 bp, synthetic construct
  source 1..59: mol_type = other DNA
aattcaaaaaaacattacgtcgcatagaaatgtcttgaatttctatgcgacgtaatgtt

SEQ ID NO: 79 - DNA, 59 bp, synthetic construct
  source 1..59: mol_type = other DNA
ccggaatacgtcgcatagaaatcaatcaagacttgatttctatgcgacgtatttttttg

SEQ ID NO: 80 - DNA, 59 bp, synthetic construct
  source 1..59: mol_type = other DNA
aattcaaaaaaatacgtcgcatagaaatcaagtcttgattgatttctatgcgacgtatt

SEQ ID NO: 81 - DNA, 59 bp, synthetic construct
  source 1..59: mol_type = other DNA
ccggaacgtcgcatagaaatcaatatcaagactattgatttctatgcgacgtttttttg

SEQ ID NO: 82 - DNA, 59 bp, synthetic construct
  source 1..59: mol_type = other DNA
aattcaaaaaaacgtcgcatagaaatcaatagtcttgatattgatttctatgcgacgtt

SEQ ID NO: 83 - DNA, 19 bp, synthetic construct
  source 1..19: mol_type = other DNA
cgagtactgctcaatgacg

SEQ ID NO: 84 - DNA, 19 bp, synthetic construct
  source 1..19: mol_type = other DNA
gcgagtactgctcaatgac

SEQ ID NO: 85 - DNA, 19 bp, synthetic construct
  source 1..19: mol_type = other DNA
tgcgagtactgctcaatga

SEQ ID NO: 86 - DNA, 19 bp, synthetic construct
  source 1..19: mol_type = other DNA
ctgcgagtactgctcaatg

SEQ ID NO: 87 - DNA, 19 bp, synthetic construct
  source 1..19: mol_type = other DNA
cctgcgagtactgctcaat

SEQ ID NO: 88 - DNA, 19 bp, synthetic construct
  source 1..19: mol_type = other DNA
ccctgcgagtactgctcaa

SEQ ID NO: 89 - DNA, 19 bp, synthetic construct
  source 1..19: mol_type = other DNA
gccctgcgagtactgctca

SEQ ID NO: 90 - DNA, 19 bp, synthetic construct
  source 1..19: mol_type = other DNA
ggccctgcgagtactgctc

SEQ ID NO: 91 - DNA, 19 bp, synthetic construct
  source 1..19: mol_type = other DNA
tggccctgcgagtactgct

SEQ ID NO: 92 - DNA, 19 bp, synthetic construct
  source 1..19: mol_type = other DNA
gtggccctgcgagtactgc

SEQ ID NO: 93 - DNA, 19 bp, synthetic construct
  source 1..19: mol_type = other DNA
ggtggccctgcgagtactg

SEQ ID NO: 94 - DNA, 19 bp, synthetic construct
  source 1..19: mol_type = other DNA
aggtggccctgcgagtact

SEQ ID NO: 95 - DNA, 19 bp, synthetic construct
  source 1..19: mol_type = other DNA
gaggtggccctgcgagtac

SEQ ID NO: 96 - DNA, 19 bp, synthetic construct
  source 1..19: mol_type = other DNA
tgaggtggccctgcgagta

SEQ ID NO: 97 - DNA, 19 bp, synthetic construct
  source 1..19: mol_type = other DNA
ttgaggtggccctgcgagt

SEQ ID NO: 98 - DNA, 19 bp, synthetic construct
  source 1..19: mol_type = other DNA
gttgaggtggccctgcgag

SEQ ID NO: 99 - DNA, 19 bp, synthetic construct
  source 1..19: mol_type = other DNA
ggttgaggtggccctgcga

SEQ ID NO: 100 - DNA, 19 bp, synthetic construct
  source 1..19: mol_type = other DNA
aggttgaggtggccctgcg

SEQ ID NO: 101 - DNA, 19 bp, synthetic construct
  source 1..19: mol_type = other DNA
gaggttgaggtggccctgc

SEQ ID NO: 102 - DNA, 19 bp, synthetic construct
  source 1..19: mol_type = other DNA
tgagtactgctcaatgacg

SEQ ID NO: 103 - DNA, 19 bp, synthetic construct
  source 1..19: mol_type = other DNA
gtgagtactgctcaatgac

SEQ ID NO: 104 - DNA, 19 bp, synthetic construct
  source 1..19: mol_type = other DNA
tgtgagtactgctcaatga

SEQ ID NO: 105 - DNA, 19 bp, synthetic construct
  source 1..19: mol_type = other DNA
ctgtgagtactgctcaatg

SEQ ID NO: 106 - DNA, 19 bp, synthetic construct
  source 1..19: mol_type = other DNA
cctgtgagtactgctcaat

SEQ ID NO: 107 - DNA, 19 bp, synthetic construct
  source 1..19: mol_type = other DNA
ccctgtgagtactgctcaa

SEQ ID NO: 108 - DNA, 19 bp, synthetic construct
  source 1..19: mol_type = other DNA
gccctgtgagtactgctca

SEQ ID NO: 109 - DNA, 19 bp, synthetic construct
  source 1..19: mol_type = other DNA
ggccctgtgagtactgctc

SEQ ID NO: 110 - DNA, 19 bp, synthetic construct
  source 1..19: mol_type = other DNA
tggccctgtgagtactgct

SEQ ID NO: 111 - DNA, 19 bp, synthetic construct
  source 1..19: mol_type = other DNA
gtggccctgtgagtactgc

SEQ ID NO: 112 - DNA, 19 bp, synthetic construct
  source 1..19: mol_type = other DNA
ggtggccctgtgagtactg

SEQ ID NO: 113 - DNA, 19 bp, synthetic construct
  source 1..19: mol_type = other DNA
aggtggccctgtgagtact

SEQ ID NO: 114 - DNA, 19 bp, synthetic construct
  source 1..19: mol_type = other DNA
gaggtggccctgtgagtac

SEQ ID NO: 115 - DNA, 19 bp, synthetic construct
  source 1..19: mol_type = other DNA
tgaggtggccctgtgagta

SEQ ID NO: 116 - DNA, 19 bp, synthetic construct
  source 1..19: mol_type = other DNA
ttgaggtggccctgtgagt

SEQ ID NO: 117 - DNA, 19 bp, synthetic construct
  source 1..19: mol_type = other DNA
gttgaggtggccctgtgag

SEQ ID NO: 118 - DNA, 19 bp, synthetic construct
  source 1..19: mol_type = other DNA
ggttgaggtggccctgtga

SEQ ID NO: 119 - DNA, 19 bp, synthetic construct
  source 1..19: mol_type = other DNA
aggttgaggtggccctgtg

SEQ ID NO: 120 - DNA, 19 bp, synthetic construct
  source 1..19: mol_type = other DNA
gaggttgaggtggccctgt

SEQ ID NO: 121 - DNA, 21 bp, synthetic construct
  source 1..21: mol_type = other DNA
tgaggatgctacacgtcgacc

SEQ ID NO: 122 - DNA, 23 bp, synthetic construct
  source 1..23: mol_type = other DNA
agccgatgtacagggtggttatc

SEQ ID NO: 123 - DNA, 21 bp, synthetic construct
  source 1..21: mol_type = other DNA
tgaggatgctacacgtcgact

SEQ ID NO: 124 - DNA, 23 bp, synthetic construct
  source 1..23: mol_type = other DNA
agccgatgtacagggtggttatc

SEQ ID NO: 125 - DNA, 20 bp, synthetic construct
  source 1..20: mol_type = other DNA
tcctgaggatgctacacgtc

SEQ ID NO: 126 - DNA, 23 bp, synthetic construct
  source 1..23: mol_type = other DNA
agccgatgtacagggtggttatc

SEQ ID NO: 127 - DNA, 22 bp, synthetic construct
  source 1..22: mol_type = other DNA
atcacattcacatgggtggagc

SEQ ID NO: 128 - DNA, 25 bp, synthetic construct
  source 1..25: mol_type = other DNA
acagatacttcaggggattctcagg

SEQ ID NO: 129 - DNA, 20 bp, synthetic construct
  source 1..20: mol_type = other DNA
catccgcctagtcaagcact

SEQ ID NO: 130 - DNA, 21 bp, synthetic construct
  source 1..21: mol_type = other DNA
aagaccgtccgaaatccctgg

SEQ ID NO: 131 - DNA, 21 bp, synthetic construct
  source 1..21: mol_type = other DNA
acccactcctccacctttgac

SEQ ID NO: 132 - DNA, 22 bp, synthetic construct
  source 1..22: mol_type = other DNA
accctgttgctgtagccaaatt

SEQ ID NO: 133 - DNA, 21 bp, synthetic construct
  source 1..21: mol_type = other DNA
tgacactggcaaaacaatgca

SEQ ID NO: 134 - DNA, 21 bp, synthetic construct
  source 1..21: mol_type = other DNA
ggtccttttcaccagcaagct

SEQ ID NO: 135 - DNA, 21 bp, synthetic construct
  source 1..21: mol_type = other DNA
gctcacccaccaacaatttag

SEQ ID NO: 136 - DNA, 22 bp, synthetic construct
  source 1..22: mol_type = other DNA
tctgctctgactttagcacctg

SEQ ID NO: 137 - DNA, 23 bp, synthetic construct
  source 1..23: mol_type = other DNA
agaatagcaatggtgtgacgcag

SEQ ID NO: 138 - DNA, 22 bp, synthetic construct
  source 1..22: mol_type = other DNA
tggatgttctgggtctggttgc

SEQ ID NO: 139 - DNA, 20 bp, synthetic construct
  source 1..20: mol_type = other DNA
tggttggagggaaggtgaag

SEQ ID NO: 140 - DNA, 21 bp, synthetic construct
  source 1..21: mol_type = other DNA
tgtctatctactgtgtcccag

SEQ ID NO: 141 - DNA, 20 bp, synthetic construct
  source 1..20: mol_type = other DNA
tgtactcctcggtccctttc

SEQ ID NO: 142 - DNA, 19 bp, synthetic construct
  source 1..19: mol_type = other DNA
caggtggggtctttcattc

SEQ ID NO: 143 - DNA, 21 bp, synthetic construct
  source 1..21: mol_type = other DNA
accaatcccatccacactcac

SEQ ID NO: 144 - DNA, 22 bp, synthetic construct
  source 1..22: mol_type = other DNA
tctatacaaggtccattccccc

SEQ ID NO: 145 - DNA, 19 bp, synthetic construct
  source 1..19: mol_type = other DNA
atcccagtgtggtggtacg

SEQ ID NO: 146 - DNA, 21 bp, synthetic construct
  source 1..21: mol_type = other DNA
aaggcattcatgggccgcttg

SEQ ID NO: 147 - DNA, 21 bp, synthetic construct
  source 1..21: mol_type = other DNA
acaactgacaccaacacctgc

SEQ ID NO: 148 - DNA, 22 bp, synthetic construct
  source 1..22: mol_type = other DNA
agtccaagttgccagtcacgtc

SEQ ID NO: 149 - DNA, 20 bp, synthetic construct
  source 1..20: mol_type = other DNA
tctatccacgtgcctacagc

SEQ ID NO: 150 - DNA, 21 bp, synthetic construct
  source 1..21: mol_type = other DNA
agaaagtcaggctggctcaag

SEQ ID NO: 151 - DNA, 21 bp, synthetic construct
  source 1..21: mol_type = other DNA
ctgcggcaaaacctacacaaa

SEQ ID NO: 152 - DNA, 22 bp, synthetic construct
  source 1..22: mol_type = other DNA
ttatcgtcgaccactgtgctgg

SEQ ID NO: 153 - DNA, 21 bp, synthetic construct
  source 1..21: mol_type = other DNA
acctgtccaagttccgcaagg

SEQ ID NO: 154 - DNA, 22 bp, synthetic construct
  source 1..22: mol_type = other DNA
ttacaggttggcaagagtgagg

SEQ ID NO: 155 - DNA, 21 bp, synthetic construct
  source 1..21: mol_type = other DNA
acctgtccaagttccgcaagg

SEQ ID NO: 156 - DNA, 19 bp, synthetic construct
  source 1..19: mol_type = other DNA
tttgctggcacctccaggg

SEQ ID NO: 157 - DNA, 35 bp, synthetic construct
  source 1..35: mol_type = other DNA
gtcattgagcagtactcacagggccacctcaacct

SEQ ID NO: 158 - DNA, 35 bp, synthetic construct
  source 1..35: mol_type = other DNA
gtcattgagcagtactcgcagggccacctcaacct

SEQ ID NO: 159 - DNA, 19 bp, synthetic construct
  source 1..19: mol_type = other DNA
aggttgaggtcgccctgtg

SEQ ID NO: 160 - DNA, 19 bp, synthetic construct
  source 1..19: mol_type = other DNA
aggttgaggaggccctgtg

SEQ ID NO: 161 - DNA, 19 bp, synthetic construct
  source 1..19: mol_type = other DNA
aggttgaggtggccctgtg

SEQ ID NO: 162 - DNA, 19 bp, synthetic construct
  source 1..19: mol_type = other DNA
ggtgcccctgcgagtactg

SEQ ID NO: 163 - DNA, 19 bp, synthetic construct
  source 1..19: mol_type = other DNA
ggtgcgcctgcgagtactg

SEQ ID NO: 164 - DNA, 19 bp, synthetic construct
  source 1..19: mol_type = other DNA
gtcatgagcgtcgcggtgg

SEQ ID NO: 165 - DNA, 19 bp, synthetic construct
  source 1..19: mol_type = other DNA
ggtggccctgcgagtactg

SEQ ID NO: 166 - DNA, 19 bp, synthetic construct
  source 1..19: mol_type = other DNA
aggtcgccctgcgagtact

SEQ ID NO: 167 - DNA, 19 bp, synthetic construct
  source 1..19: mol_type = other DNA
aggtgcccctgcgagtact

SEQ ID NO: 168 - DNA, 19 bp, synthetic construct
  source 1..19: mol_type = other DNA
aggtgggcctgcgagtact

SEQ ID NO: 169 - DNA, 19 bp, synthetic construct
  source 1..19: mol_type = other DNA
aggtggccctgcgagtact

SEQ ID NO: 170 - DNA, 19 bp, synthetic construct
  source 1..19: mol_type = other DNA
aggttgaggaggccctgcg

SEQ ID NO: 171 - DNA, 19 bp, synthetic construct
  source 1..19: mol_type = other DNA
aggttgaggtcgccctgcg

SEQ ID NO: 172 - DNA, 19 bp, synthetic construct
  source 1..19: mol_type = other DNA
aggttgaggtggccctgcg

SEQ ID NO: 173 - DNA, 12 bp, synthetic construct
  source 1..12: mol_type = other DNA
aattcaaaaaaa

(19) United States
(12) Patent Application Publication
(10) Pub. No.: US 2025/0215436 A1
(43) Pub. Date: Jul. 3, 2025

(54) Antisense Nucleic Acids for Use in the Treatment for KCNQ1 Mutation Carriers

(71) Applicant: Stichting Amsterdam UMC, Amsterdam (NL)

(72) Inventors: Yigal-Martin Pinto, Utrecht (NL); Anke Johanna Marina Tijsen, Kockengen (NL); Lucia Cocera Ortega, Utrecht (NL)

(21) Appl. No.: 18/851,933

(22) PCT Filed: Mar. 30, 2023
(86) PCT No.: PCT/NL2023/050167

§ 371 Date: Sep. 27, 2024

(30) Foreign Application Priority Data
Mar. 30, 2022 (EP) 22165681.2

(51) Int. Cl. C12N 15/113

(52) U.S. Cl. CPC C12N 15/1138; C12N 2310/11; C12N 2310/531

(57) ABSTRACT

The invention relates to isolated antisense molecule capable of inhibiting the expression of the KCNQ1 gene in a mammalian cell, wherein said antisense molecule comprises an anti-sense nucleic acid strand which is substantially complementary to a target region of a transcript encoded by the KCNQ1 gene, wherein said antisense nucleic acid strand is at least complementary to SNP rs1057128, rs8234 or rs17215465 in said target region.

### FIELD

The invention is in the technical field of treatment of heart diseases. In particular the invention relates to antisense molecules suitable for the treatment of heart diseases. More in particular, the invention relates to siRNAs and shRNAs suitable for use in the treatment of cardiac channelopathies, in particular of the Congenital long QT syndrome (LQTS) and the congenital short QT syndrome, in particular of the Long QT syndrome type 1 and short QT syndrome type 2.

### BACKGROUND OF THE INVENTION

Congenital long QT syndrome (LQTS) is the most common cardiac channelopathy with a prevalence of 1:2500 healthy live births [1]. LQTS is characterized by a prolonged ventricular action potential duration (APD) at the cellular level and a prolonged QTc interval on the electrocardiogram (ECG). LQTS can result in life-threatening arrhythmias as a result of the impaired ventricular repolarization.

Congenital LQTS is caused by mutations in genes encoding ion-channel proteins and membrane adaptor proteins. Pathogenic mutations in KCNQ1, KCNH2, and SCN5A cause long-QT syndrome types 1, 2 and 3 (LQT1, LQT2, and LQT3) respectively, and account for 97% of patients with genetically confirmed LQTS [2]. The most prevalent form is LQT1, with an occurrence of 40-55% of genetically confirmed LQTS [3]. LQT1 is caused by mutations in the KCNQ1-encoded α-subunit of the Kv7.1 potassium channel responsible for the slow component of the delayed rectifier current (IKs) during the repolarization phase of the working myocardial action potential [4].

LQT1 is effectively treated with β-blockers, even in patients with a genetic diagnosis but normal QTc [5,6]. However, intolerance and refractoriness to therapy or noncompliance to the treatments remain an issue and life-threatening arrhythmias might still occur. When β-blocker medication fails or is ill tolerated, invasive measures such as implantation of cardioverter-defibrillators (ICDs) and/or Left Cardiac Sympathetic Denervation (LCSD) are indicated [7]. This highlights the need for other, more effective therapeutic approaches, which may be found in more directly targeting the mutant protein itself.

### SUMMARY OF THE INVENTION

The invention provides an isolated antisense molecule capable of reducing or inhibiting the expression of the KCNQ1 gene, preferably an allele of the KCNQ1 gene, in a mammalian cell, said antisense molecule comprises an antisense nucleic acid strand which is substantially complementary to a target region of a transcript encoded by the KCNQ1 gene, wherein said antisense nucleic acid strand is at least complementary to SNP rs1057128, rs8234 or rs17215465 in said target region.

In a preferred embodiment, the isolated antisense molecule is selected from:
a. a double stranded nucleic acid (dsNA) or a chemically modified version thereof, or
b. an antisense oligonucleotide (ASO)

Preferably, said dsNA is RNA, more preferably a short hairpin (shRNA) or a short interfering RNA (siRNA). Preferably, said dsNA is dsRNA.

In a preferred embodiment of the isolated antisense molecule according to the invention, said antisense nucleic acid strand is of 15-30 nucleotides in length. Preferably, said antisense nucleic acid strand is complementary to at least 7, more preferably 8, 9, 10, 11, 12, 13, 14, 15, 16, 17, 18, 19, 20 or 21 nucleotides in said target region. In some embodiments there may be one or two mismatches between the antisense nucleic acid strand and the target region. Preferably, said antisense nucleic acid strand is complementary to at least 7, more preferably 8, 9, 10, 11 or 12 consecutive nucleotides. Preferably, said target region comprises at least 15, 16, 17, 18 or 19 nucleotides in said target region.

In a preferred embodiment of the isolated antisense molecule according to the invention, said target region comprises the nucleic acid sequence selected from the group consisting of: CGUCAUUGAGCAGUACUCGCAGGGCCACCUCAACCUC (SEQ ID NO:1), and CGUCAUUGAGCAGUACUCACAGGGCCACCUCAACCUC (SEQ ID NO:2), or a sequence being at least 80% homologous thereto. More preferably, at least 81, 82, 83, 84, 85, 86, 87, 88, 89, 90, 91, 92, 93, 94, 95, 96, 97, 98, 99% homologous thereto.

In a preferred embodiment of the isolated antisense molecule according to the invention, said antisense nucleic acid strand comprises a nucleic acid sequence having a consecutive strand of at least 12, 13, 14, 15 16, 17, 18 or 19 nucleotides selected from SEQ ID NO:3 or a nucleic acid analogue sequence thereof and SEQ ID NO: 4 or a nucleic acid analogue sequence thereof.

In a preferred embodiment, the antisense molecule comprises an antisense nucleic acid strand having the nucleic acid sequence according to any of SEQ ID NO: 83-120 or a nucleic acid analogue sequence thereof. More preferably the antisense molecule comprises an antisense nucleic acid strand having the nucleic acid sequence according to of SEQ ID NO: 93 and 119 or a nucleic acid analogue sequence thereof. An advantage thereof is that these antisense molecules are capable of reducing or even inhibiting the expression of only one allele of the KCNQ1 gene.

In a preferred embodiment of the isolated double stranded nucleic acid (dsNA) according to the invention, the dsNA comprises a sense nucleic acid strand of 15-30 nucleotides in length; and the sense nucleic acid strand is complementary to said antisense nucleic acid strand and the sense and antisense nucleic acid strands form a duplex region. Preferably, said dsNA is a shRNA containing an antisense nucleic acid strand of RNA of 15 to 30 nucleotides, preferably 19 nucleotides in length, having a 5′ end and a 3′ end, wherein the antisense nucleic acid strand is preferably complementary to at least 15 nucleotides of said target region, and wherein preferably the 5′ end of the sense strand of RNA is operably linked to a G nucleotide to form a first segment of RNA, and a sense strand of RNA of 15 to 30 nucleotides in length having a 5′ end and a 3′ end, wherein preferably at least 12 nucleotides of the antisense and sense strands are complementary to each other and preferably form a small interfering RNA (siRNA) duplex under physiological conditions.

Preferably, the antisense strand and the sense strand are operably linked by means of an RNA loop strand to form a hairpin structure comprising a duplex structure and a loop structure. Preferably, the loop structure preferably contains from 4 to 13, more preferably between 4 and 10 nucleotides. In an embodiment, said loop structure contains 4, 5 or 6 nucleotides. Preferably said loop contains the sequence UCAAGAC.

The duplex formed by the two strands of RNA may be between 15 and 25 base pairs in length, preferably 19 base pairs in length. The antisense strand preferably is 19 nucleotides in length. The sense strand preferably is 19 nucleotides in length. The shRNA may further contain an overhang region. Such an overhang may be a 3′ overhang region or a 5′ overhang region. The overhang region may be, for example, from 1 to 6 nucleotides in length.

The invention further provides a nucleic acid encoding the isolated antisense molecule of the invention. In a preferred embodiment, said shRNA of the invention is encoded by an oligonucleotide having the following structure: forward 5′-CCGGAA-19 bp sense strand-TCAAGAC-19 bp antisense strand-TTTTTTTG-3′ and/or reverse 5′-AATTCAAAAAAA-19 bp sense strand-GTCTTGA-19 bp antisense strand-TT-3′. Herein the sense strand has the same sequence as the targeted sequence in the mRNA and the antisense strand has its reverse complementary sequence that will eventually bind the mRNA and induce its breakdown and/or inhibition. In a preferred embodiment, said nucleic acid encoding the isolated antisense molecule of the invention comprises the nucleic acid sequence according to any of SEQ ID NO: 5-52.

The invention further provides an expression cassette comprising a nucleic acid encoding said antisense nucleic acid strand of the invention. The expression cassette may further contain a promoter. Such promoters can be regulatable promoters or constitutive promoters. Examples of suitable promoters include a CMV, RSV, pol II or pol III promoter. The expression cassette may further contain a polyadenylation signal, such as a synthetic minimal polyadenylation signal. The expression cassette may further contain a marker gene.

The invention further provides an expression vector comprising the expression cassette of the invention. Further, the vector may contain two expression cassettes, a first expression cassette containing a nucleic acid encoding the antisense strand of the RNA duplex and a second expression cassette containing a nucleic acid encoding a sense strand of the RNA duplex. Examples of appropriate vectors include adenoviral, lentiviral, adeno-associated viral (AAV), poliovirus, HSV, or murine Maloney-based viral vectors. In a preferred embodiment, the vector is a lentiviral vector. In another preferred embodiment, the vector is an AAV. Preferably said expression cassette encodes any of the antisense molecules as described above.

The invention further provides a cell containing an expression cassette according to the invention. Preferably said cell is a mammalian cell, preferably a human cell. Preferably, said cell is a cardiomyocyte. In certain embodiments, said cell is a Human induced pluripotent stem cell-derived cardiomyocyte (hiPSC-CM).

The invention further provides a pharmaceutical composition comprising the antisense molecule according to the invention, the isolated antisense molecule of the invention, or the nucleic acid encoding said isolated antisense molecule of the invention, and a pharmaceutically acceptable carrier.

The invention further provides the isolated antisense molecule according to the invention, the nucleic acid encoding the isolated antisense molecule, the expression cassette of the invention, the expression vector of the invention, or the pharmaceutical composition according to the invention for use in a medical treatment.

The invention further provides the isolated antisense molecule according to the invention, the nucleic acid encoding the isolated antisense molecule of the invention, the expression cassette of the invention, or the expression vector of the invention, the pharmaceutical composition according to the invention for use in the treatment of a cardiac arrhythmia. In a preferred embodiment said treatment is of 20 long QT syndrome or short QT syndrome, preferably of long QT syndrome type 1.

The invention further provides a method for selecting an antisense molecule which is suitable for the medical treatment of a disease caused by an autosomal dominant mutation present on a mutant allele of a gene, said method comprising:
a. Providing a nucleic acid sample of a subject suffering from a disease which is caused by an autosomal dominant mutation present on a mutant allele of a gene,
b. Determining the heterozygosity of the mutant allele of said gene which is responsible for said disease,
c. Determining the presence of an heterozygous SNP in said gene,
d. Determining the nucleic acid sequence of a target region comprising the heterozygous SNP of the mutant allele of said gene,
e. Selecting an isolated antisense molecule which is complementary to said target nucleic acid strand.

In a preferred embodiment, said antisense molecule is as described herein. Preferably, said gene is the KCNQ1 gene. Preferably, said SNP is selected from rs1057128, rs8234 and rs17215465. Preferably, said disease is a heart failure, preferably a cardiac arrhythmia, more preferably of long QT syndrome, more preferably of long QT syndrome type 1.

The invention further provides an in vitro method for inhibiting the expression of the KCNQ1 gene in a cell, comprising the following steps:
introducing into the cell an antisense molecule of the invention which, upon contact with a cell expressing a KCNQ1 mutant, inhibits expression of the KCNQ1 mutant allele by at least 20%; and
maintaining the cell produced in step (a) for a time sufficient to obtain degradation of the mRNA transcript of the KCNQ1 mutant allele, thereby inhibiting expression of the KCNQ1 mutant allele in the cell.

### BRIEF DESCRIPTION OF THE FIGURES

FIG. 1 shows allele specific downregulation of KCNQ1 expression in hiPSC-CMs by shRNAs targeting SNP rs1057128. 1A) Schematic representation of the shRNAs targeting the A allele of rs1057128 on the mutant KCNQ1 allele in this hiPSC line. 1B) Schematic representation of the siRNA sequences targeting the A allele of rs1057128 with the mismatch positions indicated in bold. Middle, allele-specific relative mRNA expression of the wild-type (1C) and mutant allele (1D) and total KCNQ1 (1E) in hiPSC-CM of Line 1 (n=6). Bottom, allele-specific relative mRNA expression of the wild-type (1F) and mutant allele (1G) and total KCNQ1 (1H) in hiPSC-CM of Line 2 (n=6). 1I) Schematic representation of the shRNAs targeting the G allele of SNP rs1057128 on the wild-type KCNQ1 allele in this hiPSC line. 1J) Schematic representation of the siRNA sequences targeting the G allele of rs1057128 with the mismatch positions indicated in bold. Middle, allele-specific relative mRNA expression of the wild-type (1K) and mutant (1L) allele and total KCNQ1 (1M) in hiPSC-CM of Line 1 (n=12). Bottom, allele-specific relative mRNA expression of the wild-type (1N) and mutant (10) allele and total KCNQ1 (1P) in hiPSC-CM of Line 2 (n=6). *P<0.05; **P<0.025; ***P<0.001 compared to shSCR negative control shRNA; error bars indicate SEM.

FIG. 2 shows the allelic imbalance induced by allele-specific shRNAs targeting SNP rs10571287. 2A) Schematic representation of the shRNAs targeting A allele of SNP rs1057128 on the mutant KCNQ1 allele in hiPSC-CMs of this line with the corresponding siRNA sequences in 2B. Below, allelic expression of wild-type and mutant KCNQ1 allele presented as % of total KCNQ1 expression for Line 1 (2C; n=6) and Line 2 (2D; n=6). 2E) Schematic representation of the shRNAs targeting the G allele of rs1057128 on the wild-type KCNQ1 allele in these hiPSC-lines with the corresponding siRNA sequences in 2F. Below, allelic expression of wild-type and mutant KCNQ1 allele presented as % of total KCNQ1 expression for Line 1 (2G; n=12) and Line 2 (2H; n=6). *P<0.05; **P<0.025; ***P<0.001 compared to allelic expression in the shSCR negative control shRNA; error bars indicate SEM.

FIG. 3 shows that the action potential duration is affected by shifts in allelic balance. 3A) schematic representation of SNP rs1057128 and mutation in KCNQ1 in the hiPSC-lines used in this study. Furthermore, typical recordings of optical action potentials derived from ArcLight fluorescence changes in hiPSC-CMs from Line 1 (3B) or Line 2 (3F) treated with either negative control shSCR, shA18 targeting the mutant KCNQ1 allele and shG11 targeting the wild-type KCNQ1 allele stimulated at 1 Hz Right, action potential duration at 20 (3C/G), 50 (3D/H) or 80% (3E/I) of repolarization (APD20, APD50, and APD80, respectively) of optical action potentials of Line 1 (C/D/E) or Line 2 (G/H/I). *P<0.05; **P<0.025; ***P<0.001 compared to shSCR negative control treated hiPSC-CMs; error bars indicate SEM.

FIG. 4 shows that the occurrence of arrhythmic events is affected by allele-specific downregulation of mutant or wild-type KCNQ1 alleles. a) Typical examples of ArcLight traces showing the changes in fluorescence over time of hiPSC-CMs displaying arrhythmic events (arrows); b) Percentage of cells with arrhythmic events in hiPSC-CM of Line 1 (shSCR: n=28, 2 with events; shA18: n=19, no events; shG11: n=34, 19 with events); and c) in hiPSC-CM Line 2 (shSCR: n=43, 5 with events; shA18: n=47, 4 with events; shG11: n=41, 21 with events).

FIG. 5 shows that the introduction of additional mismatches does not improve allele-specificity of shRNAs targeting the A allele of SNP rs1057128 on the mutant KCNQ1 allele. 6A/B) Relative Mrna expression of interferon response genes STAT1 and OAS1 for the most efficient shRNAs targeting the A allele of rs1057128 in hiPSC-CM of Line 1 (6A) and Line 2 (6B). C) Schematic representation of the shRNA targeting the A allele of rs1057128 on the mutant KCNQ1 allele in these hiPSC lines with the corresponding siRNA sequences in 6D with the mismatch positions indicated. Furthermore, allele-specific relative mRNA expression of the wild-type (E/H) and mutant (F/I) KCNQ1 alleles and the allelic expression of the wild-type and mutant KCNQ1 alleles presented as % of total KCNQ1 expression (G/J). hiPSC-CM of Line 1 in E/F/G (n=3-9) and Line 2 in H/I/J (n=3-6). *P<0.05; **P<0.025; ***P<0.001 compared to shSCR negative control shRNA. Indicated are mean and sem.

FIG. 6 shows that introduction of additional mismatches does not improve allele-specificity of shRNAs targeting the G allele of SNP rs1057128 on the wild-type KCNQ1 allele. A/B) Relative mRNA expression of interferon response genes STAT1 and OAS1 for the most efficient shRNAs targeting the G allele of rs1057128 in hiPSC-CM of Line 1 (A) and Line 2 (B). C/K/S) Schematic representation of the shRNAs targeting the G allele of rs1057128 on the wild-type KCNQ1 allele with the corresponding siRNA sequences with mismatch positions indicated in D/L/T. Graphs show allele-specific relative mRNA expression of the wild-type (E/H/M/P/U/X) and mutant (F/I/N/Q/V/Y) KCNQ1 alleles and the allelic expression of the wild-type and mutant KCNQ1 alleles presented as % of total KCNQ1 expression (G/J/O/R/W/Z) for shG11 with the mismatches (C-J), shG12 with the mismatches (K-R) and shG18 with mismatches (S-Z). hiPSC-CM of Line 1 in E/F/G/M/N/O/U/V/W) (n=3-9) and hiPSC-CM of Line 2 in H/I/J/P/Q/R/X/Y/Z (n=3-6). *P<0.05; **P<0.025; ***P<0.001 compared to shSCR negative control; error bars indicate SEM.

FIG. 7 shows allele specific shRNAs targeting SNP rs8234 in the 3′UTR of KCNQ1 in line 2. A/E) Representation of the targeting of the A allele of rs8234 on the mutant KCNQ1 allele in hiPSC-CM of Line 2. B-D) relative allele-specific expression of the wild-type (B) and mutant (C) KCNQ1 allele and total (D) KCNQ1 (n=6). E) Representation of the targeting of the G allele of rs8234 on the wild-type KCNQ1 allele. F-H) relative allele-specific expression of the wild-type (F) and mutant (G) KCNQ1 allele and total (H) KCNQ1 measured by RT-qPCR (n=3). *P<0.05; **P<0.025; ***P<0.001 compared to shSCR negative control; error bars indicate SEM.

### DETAILED DESCRIPTION OF THE INVENTION

The present invention is based on the surprising finding that by targeting a common SNP in the KCNQ1 gene we succeeded in selectively silencing a LQT1-causing mutant allele. By targeting a common SNP, we avoid the need to make shRNAs against every single mutation and we can apply our developed shRNAs independent of the actual disease-causing mutation as long as the patient is heterozygous for this SNP. This only requires to determine which variant of the SNP resides on the mutant KCNQ1 allele. Specifically, we designed allele-specific shRNAs to selectively target SNP rs1057128 in KCNQ1 with a MAF of 16.6% and a corresponding heterozygosity of 27%. We validated these shRNAs in hiPSC-CMs from two LQT1 patients carrying an R243C mutation in KCNQ1 and we achieved a downregulation of the targeted allele up to 60%. Furthermore, we show that this specific inhibition of the mutant KCNQ1 allele decreased the occurrence of arrhythmic events in hiPSC-CMs while inhibition of the wild-type allele had the opposite effect and increased the occurrence of arrhythmic events. This underlines that our approach allows to intentionally shift the balance between expression of the targeted and non-targeted allele as desired. In addition, computer simulations assuming a heterozygous E160K mutation in KCNQ1 show that a 60% reduction of the mutant KCNQ1 allele may substantially increase the remaining IKs and shorten the prolonged APD. Together, these data indicate the applicability of our allele-specific antisense molecules to act independent of the individual LQT1-causing mutation.

Patients heterozygous for a LQT1-causing mutation combine the translated products from normal and mutant alleles to form tetrameric channels. When both alleles are expressed at similar levels, only 6.25% of the channels will consist of purely wild-type KCNQ1 subunits. When the mutant KCNQ1 allele is downregulated, the percentage of channels consisting purely of wild-type KCNQ1 subunits will increase (a), improving repolarization capacity of the cells. This is supported by our previous study [11], where we showed that suppressive SNPs on the mutant KCNQ1 allele reduce the prolonged QTc duration and the occurrence of symptoms. This indicates that a shift in allelic balance towards less expression of a particular LQT1-causing mutant KCNQ1 protein prevents the arrhythmic substrate and life-threatening events [11]. Generally, common SNPs have a small effect on the occurrence of diseases. However, in our previous study a small effect caused by common SNPs was enough to explain part of the LQT1 disease variability and to protect mutation carriers against life-threatening events. This suggests that a stronger downregulation of the disease-causing allele could result in stronger protection against the occurrence of these life-threatening arrhythmias.

Indeed, we showed that allele-specific silencing of the mutant KCNQ1 allele targeting rs1057128 by 40-60% in hiPSC-CMs shifted the allelic imbalance towards a lower expression of the mutant KCNQ1 allele and decreased the occurrence of arrhythmic events.

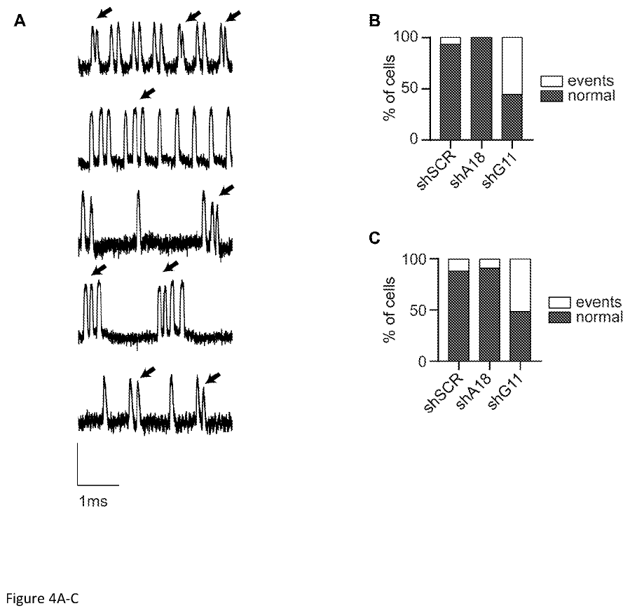

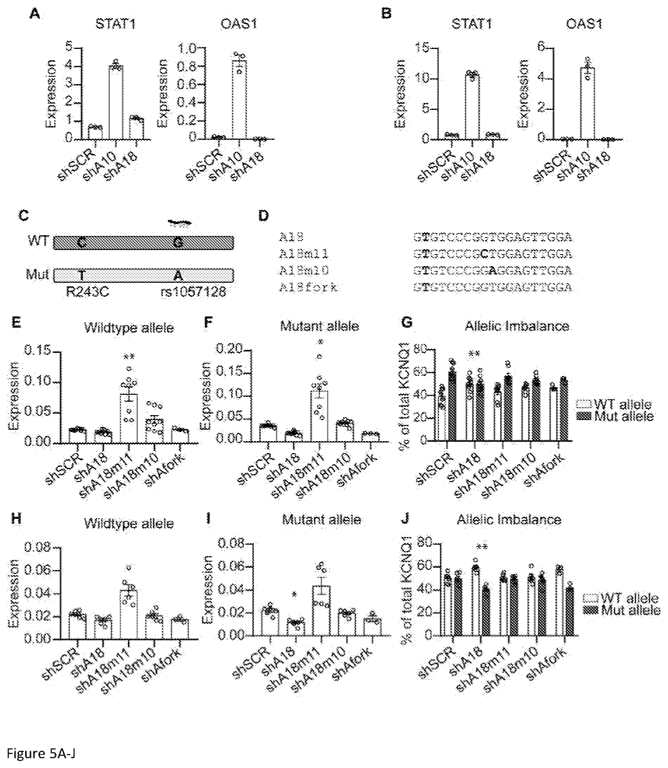

Previous studies with allele-specific siRNAs targeting mutations have shown that complete silencing of mutant mRNA is not necessary to achieve therapeutic effects. In a mouse model of hypertrophic cardiomyopathy caused by a Myh6 mutation, a reduction of 28.5% of mutant Myh6 was enough to prevent the development of HCM [24]. Furthermore, in a mouse model of autosomal dominant centronuclear myopathy (AD-CNM) treatment with allele-specific siRNAs at advanced stages of the disease reduced around 40% of the mutant allele expression and partially rescued the muscle force impairment and morphologic abnormalities [25]. We found that a reduction of 60% of mutant KCNQ1, thus increasing the number of channels consisting of only wild-type subunits from 6.25% to 26%, was enough to substantially shorten the prolonged APD in an adult human cardiomyocyte computer model of a LQT1-causing mutation in KCNQ1. We also observed that a reduction of about 60% of the mutant KCNQ1 allele was enough to decrease the number of arrhythmic events, while a reduction of about 40% of the wild-type KCNQ1 allele was enough to elicit a high number of arrhythmic events in hiPSC-CMs carrying the R243C mutation in KCNQ1 (FIG. 4). These results suggest that an allele-specific reduction of 40% is enough to functionally shift the allelic balance of wild-type and mutant KCNQ1 alleles in LQT1.

Surprisingly, we only observed an effect of the allelic imbalance on the occurrence of arrhythmias and not on the APD in our hiPSC-CMs. For this study, we used two hiPSC lines harboring the R243C mutation in KCNQ1. Especially the patient where Line 1 was derived from only had a borderline [6] prolonged QTc of 434 ms on the ECG. Furthermore, hiPSC-CMs of both lines do not display a long APD compared to hiPSC-CMs of previously published LQT1 lines with different mutations [17,27,28]. Effects on APD may be in the present study obscured due to the huge APD variability between hiPSC-CMs lines but could also be mutation dependent (29). In addition, hiPSC-CMs are characterized by immaturity and they lack the inward rectifier potassium current (IK1), which results in a more depolarized resting membrane potential than adult cardiomyocytes [30]. In such situation a reduction in repolarizing currents can further depolarize the resting membrane potential as has been found for reduction of the rapid delayed rectifier potassium current (IKr) reduction [31].

Despite the lack of APD changes in hiPSC-CMs, which could be explained by the immaturity of these cells, we might also detect an effect on the occurrence of arrhythmias as a result of the allelic imbalance which is independent of the effect on APD. Indeed, we observed in our previous study that an allelic imbalance by suppressive 3′UTR SNPs affected the occurrence of symptoms even when the analysis was corrected for QTc duration, which indicates that the allelic imbalance still affects the occurrence of symptoms via other mechanisms independent of the QTc duration. Furthermore, the QTc duration is not always useful for predicting serious arrhythmic events in carriers of LQTS mutations [32]. This means that patients could still benefit from inducing an allelic imbalance by allele specific shRNAs even though we do not detect shortening of the APD when we suppress the mutant KCNQ1 allele.

We targeted a common SNP to specifically inhibit the mutant KCNQ1 allele and thereby expand the applicability of our allele-specific shRNAs to a wider number of patients independent of their specific LQT1-causing mutation. There have been more than 600 mutations described in KCNQ1, which are associated with either LQT1 or much less frequently short-QT syndrome type 2 (SQT2) [33]. It would be a tremendous task to design and optimize allele-specific shRNAs for every single mutation.

Furthermore, it is questionable whether drug regulatory agencies will allow patient/family-specific shRNAs without large clinical trials, which would be impossible for patient-specific mutations. In addition, allele specificity is highly dependent on the sequence to be targeted, which could mean that some mutations will not be suitable at all for allele-specific targeting [34]. Targeting a common SNP instead of a specific mutation increases the number of patients that can be treated with a single shRNA [35,36] and thus overcomes the necessity to design hundreds of shRNAs for LQT1. Specifically, SNP rs1057128 has a heterozygosity of 27%, which means that 27% of patients (LQT1 and SQT2) could potentially be treated with our allele specific-shRNAs. Therefore, we developed allele-specific shRNAs for each allele of the SNP (G/A) and in patients heterozygous for this SNP it will only be necessary to determine which allele of the SNP resides on their mutant KCNQ1 allele. In addition, since SQT2 is caused by gain of function mutations in KCNQ1, allele-specific shRNAs targeting common SNPs in KCNQ1 could also be used to downregulate the mutant KCNQ1 protein in SQT2, which further expands the potential use of this system.

Functional siRNA duplexes can be injected as naked siRNAs with or without chemical modifications for stability. Delivery of these siRNAs to the heart could be enhanced via nanoparticle delivery and/or conjugation to peptides. Gene therapy delivery of shRNAs may be considered in targeting the human heart. The preferred vector for delivery of gene therapy to humans are the adeno-associated viruses (AAV), due to their safety profile. Currently, there have been 51 clinical trials with proven efficacy using AAV vectors [39]. The advantage of our shRNAs of 45 bp in length is that their small transgene size ensures high AAV production titers. These high titers might be needed to ensure that a high percentage of cells are hit, which is especially an important requirement for the treatment of LQT1. Targeting a low percentage of cardiomyocytes could result in heterogeneous cardiac repolarization between cells, which could also induce cardiac arrhythmias, in particular if this percentage varies throughout the ventricular tissue [40].

### I. Definitions

The term “antisense molecule” refers to an oligonucleotide molecule that contains sequence complementarity to target RNA molecules, such as mRNA, viral RNA, or other RNA species, and that inhibits the function of their target RNA after sequence-specific binding.

The term “nucleic acid” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., (1991). Nucleic Acids Res 19:5081; Ohtsuka et al., (1985). J Biol Chem 260:2605-2608; Rossolini et al., (1994). Mol Cell Probes 8:91-98).

A “nucleic acid fragment” is a portion of a given nucleic acid molecule.

The term “nucleotide sequence” refers to a polymer of DNA or RNA which can be single- or double-stranded, optionally containing synthetic, non-natural or altered nucleotide bases capable of incorporation into DNA or RNA polymers.

The terms “nucleic acid”, “nucleic acid molecule”, “nucleic acid fragment”, “nucleic acid sequence or segment”, or “polynucleotide” are used interchangeably and may also be used interchangeably with gene, cDNA, DNA and RNA encoded by a gene.

The term “nucleic acid analogue” refers to compounds which are analogous (structurally similar) to naturally occurring RNA and DNA. For example, RNA is a naturally occurring nucleic acid analogue of DNA. Nucleic acids are chains of nucleotides, which are composed of three parts: a phosphate backbone, a pentose sugar, either ribose or deoxyribose, and one of four nucleobases. An analogue may have any of these altered. Typically the analogue nucleobases confer, among other things, different base pairing and base stacking properties. Examples include universal bases, which can pair with all four canonical bases, and phosphate-sugar backbone analogues such as PNA. Artificial nucleic acids include peptide nucleic acid (PNA), Morpholino and locked nucleic acid (LNA), as well as glycol nucleic acid (GNA), threose nucleic acid (TNA) and hexitol nucleic acids (HNA). Each of these is distinguished from naturally occurring DNA or RNA by changes to the backbone of the molecule.

The term “nucleic acid analogue sequence of a nucleic acid sequence of a certain SEQ ID NO” refers to a nucleic acid sequence which is analogue to said SEQ ID NO. For instance SEQ ID NO: 83 refers to DNA sequence CGAGTACTGCTCAATGACG. The RNA analogue nucleic acid thereof has the Thymidine exchanged for Uracil. Another example of a nucleic acid analogue sequence of a nucleic acid sequence of SEQ ID NO 83 is a PNA nucleic acid sequence having the bases CGAGTACTGCTCAATGACG.

The term “chemically modified version of a nucleic acid” refers to a chemical modification of one or more nucleotides in a nucleic acid which does not deteriorate the binding of said nucleic acid to its complementary strand. A lengthy description of many known chemical modifications are set out in published PCT patent application WO 200370918 and will not be repeated here. Suitable modifications include, but are not limited to modifications to the sugar moiety (i.e. the 2′ position of the sugar moiety, such as for instance 2′-O-(2-methoxyethyl) or 2′-MOE) (Martin, Helv. Chim. Acta, 1995, 78, 486-504) i.e., an alkoxyalkoxy group) or the base moiety (i.e. a non-natural or modified base which maintains ability to pair with another specific base in an alternate nucleotide chain). Other modifications include so-called ‘backbone’ modifications including, but not limited to, replacing the phosphoester group (connecting adjacent ribonucleotides with for instance phosphorothioates, chiral phosphorothioates or phosphorodithioates).

The antisense molecules or dsNA molecules of the invention can be comprised of naturally occurring nucleotides or can be comprised of at least one modified nucleotide, such as a 2′-O-methyl modified nucleotide, a nucleotide comprising a 5′-phosphorothioate group, and a terminal nucleotide linked to a cholesteryl derivative or dodecanoic acid bisdecylamide group. Alternatively, the modified nucleotide may be chosen from the group of: a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide.

The terms “isolated and/or purified” refer to in vitro isolation of a nucleic acid, e.g., a DNA or RNA molecule from its natural cellular environment, and from association with other components of the cell, such as nucleic acid or polypeptide, so that it can be sequenced, replicated, and/or expressed. For example, “isolated nucleic acid” may be a DNA molecule containing less than 31 sequential nucleotides that is transcribed into an siRNA. Such an isolated siRNA may, for example, form a hairpin structure with a duplex 21 base pairs in length that is complementary or hybridizes to a sequence in a gene of interest, and remains stably bound under stringent conditions. Thus, the RNA or DNA is “isolated” in that it is free from at least one contaminating nucleic acid with which it is normally associated in the natural source of the RNA or DNA and is preferably substantially free of any other mammalian RNA or DNA. The phrase “free from at least one contaminating source nucleic acid with which it is normally associated” includes the case where the nucleic acid is reintroduced into the source or natural cell but is in a different chromosomal location or is otherwise flanked by nucleic acid sequences not normally found in the source cell, e.g., in a vector or plasmid.

Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein.

The term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include nonexpressed DNA segments that, for example, form recognition sequences for other proteins. “Genes” can be obtained from a variety of sources, including cloning from a source of interest or synthesizing from known or predicted sequence information, and may include sequences designed to have desired parameters.

An “allele” is one of several alternative forms of a gene occupying a given locus on a chromosome.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the invention will have at least 40%, 50%, 60%, at least 70%, e.g., 71%, 72%, 73%, 74%, 75%, 76%, 77%, 78%, 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“Conservatively modified variations” of a particular nucleic acid sequence refers to those nucleic acid sequences that encode identical or essentially identical amino acid sequences. Because of the degeneracy of the genetic code, a large number of functionally identical nucleic acids encode any given polypeptide. For instance, the codons CGT, CGC, CGA, CGG, AGA and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein that encodes a polypeptide also describes every possible silent variation, except where otherwise noted. One of skill in the art will recognize that each codon in a nucleic acid (except ATG, which is ordinarily the only codon for methionine) can be modified to yield a functionally identical molecule by standard techniques. Accordingly, each “silent variation” of a nucleic acid that encodes a polypeptide is implicit in each described sequence.

“Recombinant DNA molecule” is a combination of DNA sequences that are joined together using recombinant DNA technology and procedures used to join together DNA sequences as described, for example, in Sambrook and Russell (2001), Molecular Cloning: A Laboratory Manual. Cold Spring Harbor Laboratory Press, New York.

A “homologous” DNA or RNA sequence is a sequence that is naturally associated with a host cell into which it is introduced.

A “vector” is defined to include, inter alia, any viral vector, as well as any plasmid, cosmid, phage or binary vector in double or single stranded linear or circular form that may or may not be self-transmissible or mobilizable, and that can transform prokaryotic or eukaryotic host either by integration into the cellular genome or exist extrachromosomally (e.g., autonomous replicating plasmid with an origin of replication).

“Expression cassette” as used herein means a nucleic acid sequence capable of directing expression of a particular nucleotide sequence in an appropriate host cell, which may include a promoter operably linked to the nucleotide sequence of interest that may be operably linked to termination signals. It also may include sequences required for proper translation of the nucleotide sequence. The coding region usually codes for a protein of interest but may also code for a functional RNA of interest, for example an antisense molecule according to the invention, an antisense RNA, a nontranslated RNA in the sense or antisense direction, or a siRNA. The expression cassette including the nucleotide sequence of interest may be chimeric. The expression cassette may also be one that is naturally occurring but has been obtained in a recombinant form useful for heterologous expression. The expression of the nucleotide sequence in the expression cassette may be under the control of a constitutive promoter or of an regulatable promoter that initiates transcription only when the host cell is exposed to some particular stimulus. In the case of a multicellular organism, the promoter can also be specific to a particular tissue or organ or stage of development.

“Gene silencing” refers to the suppression of gene expression, e.g., transgene, heterologous gene and/or endogenous gene expression. Gene silencing may be mediated through processes that affect transcription and/or through processes that affect post-transcriptional mechanisms. In some embodiments, gene silencing occurs when siRNA initiates the degradation of the mRNA of a gene of interest in a sequence-specific manner via RNA interference. In some embodiments, gene silencing may be allele-specific. “Allele-specific” gene silencing refers to the specific silencing of one allele of a gene.

“RNA interference (RNAi)” is the process of sequence-specific, post-transcriptional gene silencing initiated by siRNA. RNAi is seen in a number of organisms such as Drosophila, nematodes, fungi and plants, and is believed to be involved in anti-viral defense, modulation of transposon activity, and regulation of gene expression. During RNAi, siRNA induces degradation of target mRNA with consequent sequence-specific inhibition of gene expression.

“siRNA” or “small-interfering ribonucleic acid” according to the invention has the meanings known in the art, including the following aspects. The siRNA consists of two strands of ribonucleotides which hybridize along a complementary region under physiological conditions. The strands are separate but they may be joined by a molecular linker in certain embodiments. The individual ribonucleotides may be unmodified naturally occurring ribonucleotides, unmodified naturally occurring deoxyribonucleotides or they may be chemically modified or synthetic as described elsewhere herein.

A “RNA duplex” or “dsRNA” refers to the structure formed by the complementary pairing between two regions of a RNA molecule. siRNA is “targeted” to a gene in that the nucleotide sequence of the duplex portion of the siRNA is complementary to a nucleotide sequence of the KCNQ1 gene. In some embodiments, the length of the duplex of siRNAs is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12 or 13 nucleotides in length. The hairpin structure can also contain 3′ or 5′ overhang portions. In some embodiments, the overhang is a 3′ or a 5′ overhang 0, 1, 2, 3, 4 or 5 nucleotides in length. The siRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal.

As used herein, a “nucleotide overhang” refers to the unpaired nucleotide or nucleotides that protrude from the duplex structure of a dsRNA when a 3′-end of one strand of the dsRNA extends beyond the 5′-end of the other strand, or vice versa. “Blunt” or “blunt end” means that there are no unpaired nucleotides at that end of the dsRNA, i.e., no nucleotide overhang. A “blunt ended” dsRNA is a dsRNA that is double-stranded over its entire length, i.e., no nucleotide overhang at either end of the molecule.

The term “antisense strand” refers to the strand of an antisense molecule which includes a region that is substantially complementary to a target sequence.

As used herein, the term “region of complementarity” refers to the region on the antisense strand that is substantially complementary to a sequence, for example a target sequence, as defined herein.

The term “target region” as used herein refers to a region of complementarity in a strand that is substantially complementary to a region of the antisense strand.

The term “sense strand,” as used herein, refers to the strand of a dsRNA that includes a region that is substantially complementary to a region of the antisense strand.

“Treating” as used herein refers to ameliorating at least one symptom of, curing and/or preventing the development of a disease or a condition.

As used herein the term “encoded by” means that the DNA sequence in a gene or the SEQ ID NO is transcribed into the RNA of interest.

The term “capable of inhibiting the expression of the KCNQ1 gene” refers to a process of substantial silencing of the KCNQ1 gene through RNA interference. RNA interference can be triggered by an antisense molecule, including by a double stranded nucleic acid (dsNA) or by a single stranded antisense oligonucleotide (ASO). In a preferred embodiment one allele of the KCNQ1 gene is silenced. An advantage thereof is that this enables silencing the mutant allele which alleviates the disease phenotype of many heritable forms of cardiac arrhythmias.

The term “mutant KCNQ1 allele” or “mutant KCNQ1 gene” as used herein refers to a variant of the KCNQ1 allele or gene which has a negative effect on the function of the wild type KCNQ1 allele or gene. Preferably the mutant KCNQ1 allele has a dominant negative effect of the wild type KCNQ1 gene.

As used herein, the term “KCNQ1 mutation” refers to a mutation in the KCNQ1 gene which has a negative effect, preferably a dominant negative effect and preferably causes an arrhythmia, more preferably Congenital long QT syndrome (LQTS) or congenital short QT syndrome (SQTS), more preferably long QT syndrome type 1 or short QT syndrome type 2.

The terms “antisense oligomer” or “antisense compound” or “antisense oligonucleotide” or “oligonucleotide” are used interchangeably and refer to a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid: oligomer heteroduplex within the target sequence. The cyclic subunits may be based on ribose or another pentose sugar or, in certain embodiments, a morpholino group. Also contemplated are peptide nucleic acids (PNAs), locked nucleic acids (LNAs), and 2′-O-Methyl oligonucleotides, and other antisense agents known in the art.

Such an antisense oligomer can be designed to block or inhibit translation of mRNA or to inhibit natural pre-mRNA splice processing, or induce degradation of targeted mRNAs, and may be said to be “directed to” or “targeted against” a target sequence with which it hybridizes.

As used herein “antisense oligonucleotides” (ASOs) mean agents that are unmodified or chemically modified single-stranded nucleic acid molecules (usually 15-30 nt in length), which can selectively hybridize to their target complementary sequence within mRNA through Watson-Crick base pairing. Formation of an ASO-mRNA heteroduplexes induces the effects as follows: 1) activates RNase H endonuclease or as in bacteria endoribonucleases—RNase III and RNase E—leading to degradation of the bound mRNA, and leaving the ASO intact; 2) causes translational arrest by steric hindrance of ribosomal activity; 3) inhibits mRNA splicing; 4) destabilizes pre-mRNA. Indeed, what effect will occur depends on the ASO chemical composition and location of hybridization, but the subsequent result is specific down-regulation of the target gene and protein expression.

As used herein the term “rs1057128” refers to an SNP located on chr11:2776007 (GRCh38.p13). As used herein, it also refers to the corresponding nucleotide polymorphism when it is present in the transcript as encoded by the KCNQ1 gene. See https://www.ncbi.nlm.nih.gov/snp/rs1057128?horizontal_tab=true released Apr. 9, 2021.

Disclosed herein is a strategy that results in substantial silencing of targeted genes or alleles via RNA interference. Use of this strategy results in markedly diminished in vitro and in vivo expression of targeted genes or alleles. This strategy is useful in reducing expression of targeted genes or alleles in order to model biological processes or to provide therapy for human diseases.

As used herein the term “substantial silencing” in the context of silencing an allele means that the mRNA of the targeted allele is downmodulated, preferably inhibited and/or degraded, by the presence of the introduced isolated antisense molecule, such that expression of the targeted allele is reduced by about 10% to 100% as compared to the level of expression seen when the isolated antisense molecule is not present.

Generally, when an allele is substantially silenced, it will have at least 40%, 50%, 60%, 70%, e.g., 71%, 72%, 73%, 74%, 75%, 76%, 77%, 78%, 79%, generally at least 80%, e.g., 81%-84%, at least 85%, e.g., 86%, 87%, 88%, 89%, 90%, 91%, 92%, 93%, 94%, 95%, 96%, 97%, 98%, 99% or even 100% reduction expression as compared to when the isolated antisense molecule is not present.

As used herein the term “substantially normal activity” means the level of expression of an allele or gene when the isolated antisense molecule of the invention has not been introduced to a cell.

The invention provides an isolated antisense molecule capable of reducing or inhibiting the expression of the KCNQ1 gene in a mammalian cell, said antisense molecule comprising an antisense nucleic acid strand which is substantially complementary to a target region of a transcript encoded by the KCNQ1 gene, wherein said antisense nucleic acid strand is at least complementary to SNP rs1057128, rs8234 or rs17215465 in said target region. As a result of the fact that the isolated antisense molecule contains an antisense strand which is due to its complementarity to a target region which contains SNP rs1057128 in a transcript encoded by the KCNQ1 gene, the antisense molecule is capable of inhibiting the expression of multiple variants of the KCNQ1 gene. Preferably said antisense molecule targets only one allele of the KCNQ1 gene in a cell. An advantage thereof is that the allelic imbalance may be changed by using said antisense molecule.

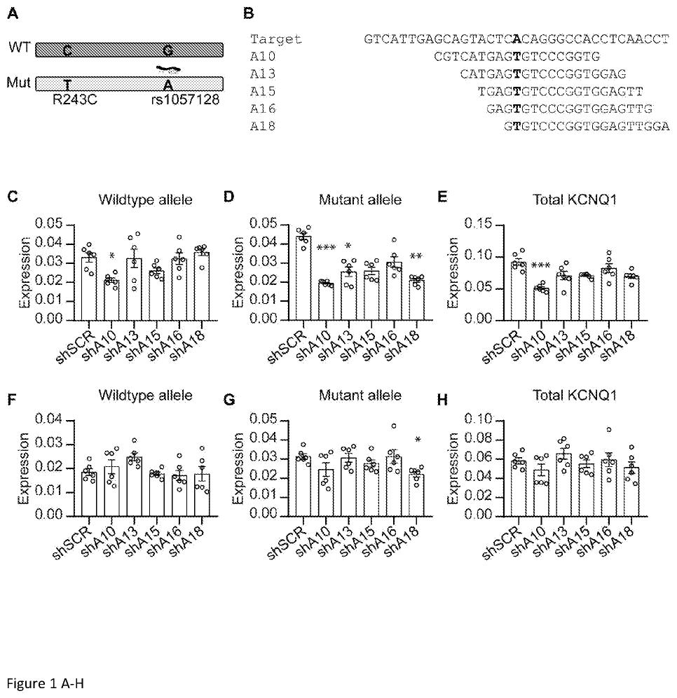

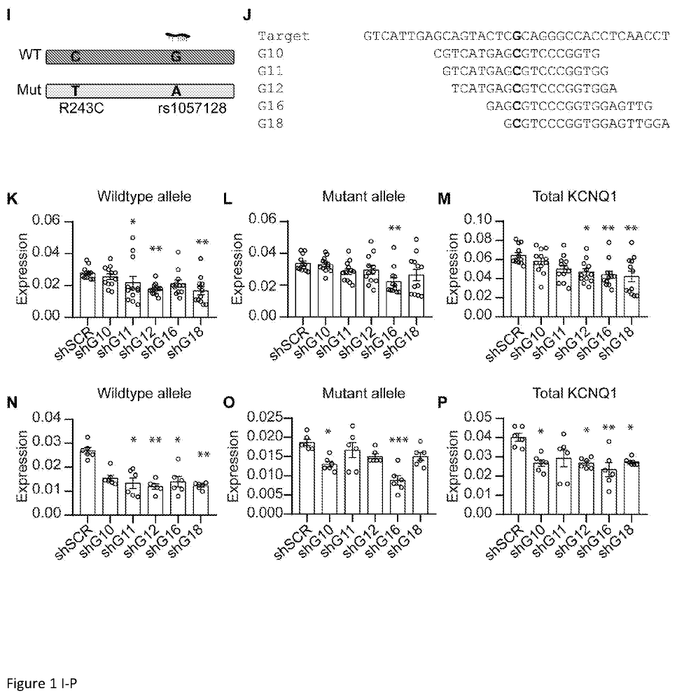

As outlined in the strategy in FIG. 1, the inventors developed siRNAs that specifically eliminate the production of the α-subunit of the Kv7.1 potassium channel. In certain preferred embodiments, these siRNAs specifically inhibit the expression of the mutant allele. By exploiting base pair differences between wild type and mutant alleles, the inventors successfully silenced expression of the mutant α-subunit of the Kv7.1 potassium channel without interfering with expression of the wild type mRNA/α-subunit of the Kv7.1 potassium channel. Because the α-subunit of the Kv7.1 potassium channel is an essential protein, silencing or inhibiting the mutant allele has obvious therapeutic potential for the treatment of diseases caused by KCNQ1 mutations, in particular cardiac channelopathies, including Congenital long QT syndrome (LQTS) and congenital short QT syndrome.

In a preferred embodiment of the isolated antisense molecule according to the invention, said antisense nucleic acid strand is of 15-30 nucleotides in length. Preferably, said antisense nucleic acid strand is complementary to at least 7, more preferably 8, 9, 10, 11, 12, 13, 14, 15, 16, 17, 18, 19, 20 or 21 nucleotides in said target region. In some embodiments there may be one or two mismatches between the antisense nucleic acid strand and the target region. Preferably, there are no more than 2 mismatches, more preferably no more than 1 mismatch. In certain preferred embodiments there is no mismatch in complementarity. Suitably, a skilled person can design mismatch positions with the highest discrimination potential depending on the type of nucleotide changes. Preferably, said antisense nucleic acid strand is complementary to at least 7, more preferably 8, 9, 10, 11 or 12 consecutive nucleotides. Preferably, said target region comprises at least 15, 16, 17, 18 or 19 nucleotides.

In a preferred embodiment, the isolated antisense molecule is selected from:
a. a double stranded nucleic acid (dsNA) or a chemically modified version thereof, or
b. an antisense oligonucleotide (ASO).

Preferably, said dsNA is RNA, more preferably a short hairpin (shRNA) or a short interfering RNA (siRNA). Preferably, said dsNA is dsRNA. Preferred embodiments of dsNA are synthetic double stranded small interfering RNA (siRNA) and vector driven short hairpin RNA (shRNA). Both siRNA and vector driven shRNA have been demonstrated to be effective in in vitro and in vivo applications, each with their respective advantages. Most siRNA are structurally designed to promote efficient incorporation into the Ago2 containing RISC, the RNase III containing Dicer-substrate design improves the efficiency of siRNA at least 10-fold by initial association and processing at the pre-RISC.

Vector driven shRNA utilizes the host microRNA biogenesis pathway, which appears to be more efficient. siRNA is more readily chemically modified while shRNA expression can be modulated and regulated by specific promoters.

In an embodiment, to accomplish intracellular expression of the therapeutic siRNA, preferably an RNA molecule is constructed containing two complementary strands or a hairpin sequence (such as a 21-bp hairpin) representing sequences directed against the target region encoded by a part of the KCNQ1 gene comprising SNP rs1057128, rs8234 or rs17215465. The siRNA, or a nucleic acid encoding the siRNA, is introduced to the target cell, such as a diseased heart cell. The siRNA reduces target mRNA and protein expression.

In an embodiment, the dsRNA of the invention is chemically modified to enhance stability. The nucleic acids of the invention may be synthesized and/or modified by methods well established in the art, such as those described in “Current protocols in nucleic acid chemistry”, Beaucage, S. L. et al. (Edrs.), John Wiley & Sons, Inc., New York, NY, USA, which is hereby incorporated herein by reference. Chemical modifications may include, but are not limited to 2′ modifications, introduction of non-natural bases, covalent attachment to a ligand, and replacement of phosphate linkages with thiophosphate linkages. In this embodiment, the integrity of the duplex structure is strengthened by at least one, and preferably two, chemical linkages. Chemical linking may be achieved by any of a variety of well-known techniques, for example by introducing covalent, ionic or hydrogen bonds; hydrophobic interactions, van der Waals or stacking interactions; by means of metal-ion coordination, or through use of purine analogues. Preferably, the chemical groups that can be used to modify the dsRNA include, without limitation, methylene blue; bifunctional groups, preferably bis-(2-chloroethyl)amine; N-acetyl-N′-(p-glyoxylbenzoyl)cystamine; 4-thiouracil; and psoralen.

In one preferred embodiment, the linker is a hexaethylene glycol linker. In this case, the dsRNA are produced by solid phase synthesis and the hexa-ethylene glycol linker is incorporated according to standard methods (e.g., Williams, D. J., and K. B. Hall, Biochem. (1996) 35:14665-14670).

In a particular embodiment, the 5′-end of the antisense strand and the 3′-end of the sense strand are chemically linked via a hexaethylene glycol linker. In another embodiment, at least one nucleotide of the dsRNA comprises a phosphorothioate or phosphorodithioate group. The chemical bond at the ends of the dsRNA is preferably formed by triple-helix bonds.

The construct encoding the therapeutic siRNA is configured such that the one or more strands of the siRNA are encoded by a nucleic acid that is immediately contiguous to a promoter. The construct is introduced into the target cell, such as by injection, allowing for diminished target-gene expression in the cell.

The present invention provides an expression cassette comprising a nucleic acid encoding at least said antisense nucleic acid strand. In some embodiments, said expression cassette comprises a nucleic acid encoding the complete isolated antisense molecule according to the invention.

In an embodiment, the siRNA of the invention may form a hairpin structure that contains a duplex structure and a loop structure. The loop structure may contain from 4 to 13, more preferably 4-10 nucleotides, such as 4, 5 or 6 nucleotides. Preferably said loop contains the sequence UCAAGAC. The duplex is less than 30 nucleotides in length, such as from 19 to 25 nucleotides. The siRNA may further contain an overhang region. Such an overhang may be a 3′ overhang region or a 5′ overhang region. The overhang region may be, for example, from 1 to 6 nucleotides in length.

The present invention also provides an expression cassette containing an isolated nucleic acid sequence encoding a first segment, a second segment located immediately 3′ of the first segment, and a third segment located immediately 3′ of the second segment, wherein the first and third segments are each less than 30 base pairs in length and each more than 10 base pairs in length, and wherein the sequence of the third segment is the complement of the sequence of the first segment, and wherein the isolated nucleic acid sequence functions as a small interfering RNA molecule (siRNA) targeted against a gene or allele of interest. The expression cassette may be contained in a vector, such as a viral vector. The expression cassette may further contain a pol II promoter, as described herein. Examples of pol II promoters include regulatable promoters and constitutive promoters. For example, the promoter may be a CMV or RSV promoter. The expression cassette may further contain a polyadenylation signal, such as a synthetic minimal polyadenylation signal. The nucleic acid sequence may further contain a marker gene. The expression cassette may be contained in a viral vector. An appropriate viral vector for use in the present invention may be an adenoviral, lentiviral, adeno-associated viral (AAV), poliovirus, herpes simplex virus (HSV) or murine Maloney-based viral vector.

The present invention provides a method of reducing the expression of a gene product, preferably an allele-specific gene product, in a cell by contacting a cell with an expression cassette described above. It also provides a method of treating a patient by administering to the patient a composition of the expression cassette described above.

The present invention further provides a method of reducing the expression of a gene product, preferably an allele-specific gene product, in a cell by contacting a cell with an expression cassette as described above.

The present invention also provides a method of treating a patient, by administering to the patient a composition containing an expression cassette according to the invention.

### II. Nucleic Acid Molecules of the Invention

Sources of nucleotide sequences from which the present nucleic acid molecules can be obtained include any vertebrate, preferably mammalian, cellular source. In addition to a DNA sequence encoding a siRNA, the nucleic acid molecules of the invention include single and double-stranded interfering RNA molecules, which are also useful to inhibit expression of a target gene, or allele thereof.

Oligonucleotide-mediated mutagenesis is a method for preparing substitution variants. This technique is known in the art. Briefly, nucleic acid encoding a siRNA can be altered by hybridizing an oligonucleotide encoding the desired mutation to a DNA template, where the template is the single-stranded form of a plasmid or bacteriophage containing the unaltered or native gene sequence. After hybridization, a DNA polymerase is used to synthesize an entire second complementary strand of the template that will thus incorporate the oligonucleotide primer, and will code for the selected alteration in the nucleic acid encoding siRNA. Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art.

The DNA template can be generated by those vectors that are either derived from bacteriophage M13 vectors (the commercially available M13 mp 18 and M13 mp 19 vectors are suitable), or those vectors that contain a single-stranded phage origin of replication. Thus, the DNA that is to be mutated may be inserted into one of these vectors to generate single-stranded template. Alternatively, single-stranded DNA template may be generated by denaturing double-stranded plasmid (or other) DNA using standard techniques.

For alteration of the native DNA sequence (to generate amino acid sequence variants, for example), the oligonucleotide is hybridized to the single-stranded template under suitable hybridization conditions. A DNA polymerizing enzyme, usually the Klenow fragment of DNA polymerase I, is then added to synthesize the complementary strand of the template using the oligonucleotide as a primer for synthesis. A heteroduplex molecule is thus formed such that one strand of DNA encodes the mutated form of the DNA, and the other strand (the original template) encodes the native, unaltered sequence of the DNA. This heteroduplex molecule is then transformed into a suitable host cell, usually a prokaryote such as E. coli JM101. After the cells are grown, they are plated onto agarose plates and screened using the oligonucleotide primer radiolabeled with 32-phosphate to identify the bacterial colonies that contain the mutated DNA. The mutated region is then removed and placed in an appropriate vector, generally an expression vector of the type typically employed for transformation of an appropriate host.

The method described immediately above may be modified such that a homoduplex molecule is created wherein both strands of the plasmid contain the mutations(s). The modifications are as follows: The single-stranded oligonucleotide is annealed to the single-stranded template as described above. A mixture of three deoxyribonucleotides, deoxyriboadenosine (dATP), deoxyriboguanosine (dGTP), and deoxyribothymidine (dTTP), is combined with a modified thiodeoxyribocytosine called dCTP-(*S) (which can be obtained from the Amersham Corporation). This mixture is added to the template-oligonucleotide complex. Upon addition of DNA polymerase to this mixture, a strand of DNA identical to the template except for the mutated bases is generated. In addition, this new strand of DNA will contain dCTP-(*S) instead of dCTP, which serves to protect it from restriction endonuclease digestion.

After the template strand of the double-stranded heteroduplex is nicked with an appropriate restriction enzyme, the template strand can be digested with ExoIII nuclease or another appropriate nuclease past the region that contains the site(s) to be mutagenized. The reaction is then stopped to leave a molecule that is only partially single-stranded. A complete double-stranded DNA homoduplex is then formed using DNA polymerase in the presence of all four deoxyribonucleotide triphosphates, ATP, and DNA ligase. This homoduplex molecule can then be transformed into a suitable host cell such as E. coli JM101.

### III. Expression Cassettes of the Invention

To prepare expression cassettes, the recombinant DNA sequence or segment may be circular or linear, double-stranded or single-stranded. Generally, the DNA sequence or segment is in the form of chimeric DNA, such as plasmid DNA or a vector that can also contain coding regions flanked by control sequences that promote the expression of the recombinant DNA present in the resultant transformed cell.

A “chimeric” vector or expression cassette, as used herein, means a vector or cassette including nucleic acid sequences from at least two different species, or has a nucleic acid sequence from the same species that is linked or associated in a manner that does not occur in the “native” or wild type of the species.

Aside from recombinant DNA sequences that serve as transcription units for an RNA transcript, or portions thereof, a portion of the recombinant DNA may be untranscribed, serving a regulatory or a structural function. For example, the recombinant DNA may have a promoter that is active in mammalian cells. Other elements functional in the host cells, such as introns, enhancers, polyadenylation sequences and the like, may also be a part of the recombinant DNA. Such elements may or may not be necessary for the function of the DNA, but may provide improved expression of the DNA by affecting transcription, stability of the siRNA, or the like. Such elements may be included in the DNA as desired to obtain the optimal performance of the siRNA in the cell.

Control sequences are DNA sequences necessary for the expression of an operably linked coding sequence in a particular host organism. The control sequences that are suitable for prokaryotic cells, for example, include a promoter, and optionally an operator sequence, and a ribosome binding site.

Eukaryotic cells are known to utilize promoters, polyadenylation signals, and enhancers. Operably linked nucleic acids are nucleic acids placed in a functional relationship with another nucleic acid sequence. For example, a promoter or enhancer is operably linked to a sequence such as a coding sequence if it affects the transcription of the sequence; or a ribosome binding site is operably linked to a coding sequence if it is positioned so as to facilitate translation. Generally, operably linked DNA sequences are DNA sequences that are linked are contiguous. However, enhancers do not have to be contiguous. Linking is accomplished by ligation at convenient restriction sites. If such sites do not exist, the synthetic oligonucleotide adaptors or linkers are used in accord with conventional practice.

The recombinant DNA to be introduced into the cells may contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. For example, reporter genes include the chloramphenicol acetyl transferase gene (cat) from Tn9 of E. coli and the luciferase gene from firefly Photinus pyralis. Expression of the reporter gene is assayed at a suitable time after the DNA has been introduced into the recipient cells.

The general methods for constructing recombinant DNA that can transfect target cells are well known to those skilled in the art, and the same compositions and methods of construction may be utilized to produce the DNA useful herein. For example, Sambrook and Russell, infra, provides suitable methods of construction. The recombinant DNA can be readily introduced into the host cells, e.g., mammalian, bacterial, yeast or insect cells by transfection with an expression vector composed of DNA encoding the siRNA by any procedure useful for the introduction into a particular cell, e.g., physical or biological methods, to yield a cell having the recombinant DNA stably integrated into its genome or existing as a episomal element, so that the DNA molecules, or sequences of the present invention are expressed by the host cell. Preferably, the DNA is introduced into host cells via a vector. The host cell is preferably of eukaryotic origin, e.g., plant, mammalian, insect, yeast or fungal sources, but host cells of non-eukaryotic origin may also be employed.

Physical methods to introduce a preselected DNA into a host cell include calcium phosphate precipitation, lipofection, particle bombardment, microinjection, electroporation, and the like. Biological methods to introduce the DNA of interest into a host cell include the use of DNA and RNA viral vectors. For mammalian gene therapy, as described hereinbelow, it is desirable to use an efficient means of inserting a copy gene into the host genome. Viral vectors, and especially retroviral vectors, have become the most widely used method for inserting genes into mammalian, e.g., human cells. Other viral vectors can be derived from poxviruses, herpes simplex virus I, adenoviruses and adeno-associated viruses, and the like.

As discussed above, a “transfected”, or “transduced” host cell or cell line is one in which the genome has been altered or augmented by the presence of at least one heterologous or recombinant nucleic acid sequence. The host cells of the present invention are typically produced by transfection with a DNA sequence in a plasmid expression vector, a viral expression vector, or as an isolated linear DNA sequence. The transfected DNA can become a chromosomally integrated recombinant DNA sequence, which is composed of sequence encoding the siRNA.

To confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

To detect and quantitate RNA produced from introduced recombinant DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species. While Southern blotting and PCR may be used to detect the recombinant DNA segment in question, they do not provide information as to whether the preselected DNA segment is being expressed. Expression may be evaluated by specifically identifying the peptide products of the introduced recombinant DNA sequences or evaluating the phenotypic changes brought about by the expression of the introduced recombinant DNA segment in the host cell.

The instant invention provides a cell expression system for expressing exogenous nucleic acid material in a mammalian recipient. The expression system, also referred to as a “genetically modified cell”, comprises a cell and an expression vector for expressing the exogenous nucleic acid material. The genetically modified cells are suitable for administration to a mammalian recipient, where they replace the endogenous cells of the recipient. Thus, the preferred genetically modified cells are non-immortalized and are non-tumorigenic.

According to one embodiment, the cells are transfected or otherwise genetically modified ex vivo. The cells are isolated from a mammal (preferably a human), nucleic acid introduced (i.e., transduced or transfected in vitro) with a vector for expressing a heterologous (e.g., recombinant) gene encoding the therapeutic agent, and then administered to a mammalian recipient for delivery of the therapeutic agent in situ. The mammalian recipient may be a human and the cells to be modified are autologous cells, i.e., the cells are isolated from the mammalian recipient.

According to another embodiment, the cells are transfected or transduced or otherwise genetically modified in vivo. The cells from the mammalian recipient are transduced or transfected in vivo with a vector containing exogenous nucleic acid material for expressing a heterologous (e.g., recombinant) gene encoding a therapeutic agent and the therapeutic agent is delivered in situ.

### VI. Delivery Vehicles for the Expression Cassettes of the Invention

The selection and optimization of a particular expression vector for expressing a specific siRNA in a cell can be accomplished by obtaining the nucleic acid sequence of the siRNA, possibly with one or more appropriate control regions (e.g., promoter, insertion sequence); preparing a vector construct comprising the vector into which is inserted the nucleic acid sequence encoding the siRNA; transfecting or transducing cultured cells in vitro with the vector construct; and determining whether the siRNA is present in the cultured cells.

Vectors for cell gene therapy include viruses, such as replication-deficient viruses. Exemplary viral vectors are derived from Harvey Sarcoma virus, ROUS Sarcoma virus, (MPSV), Moloney murine leukemia virus and DNA viruses (e.g., adenovirus).

Replication-deficient retroviruses are capable of directing synthesis of all virion proteins, but are incapable of making infectious particles. Accordingly, these genetically altered retroviral expression vectors have general utility for high-efficiency transduction of nucleic acid sequences in cultured cells, and specific utility for use in the method of the present invention. Such retroviruses further have utility for the efficient transduction of nucleic acid sequences into cells in vivo. Retroviruses have been used extensively for transferring nucleic acid material into cells. An advantage of using retroviruses for gene therapy is that the viruses insert the nucleic acid sequence encoding the siRNA into the host cell genome, thereby permitting the nucleic acid sequence encoding the siRNA to be passed on to the progeny of the cell when it divides. Promoter sequences in the LTR region have been reported to enhance expression of an inserted coding sequence in a variety of cell types. Some disadvantages of using a retrovirus expression vector are (1) insertional mutagenesis, i.e., the insertion of the nucleic acid sequence encoding the siRNA into an undesirable position in the target cell genome which, for example, leads to unregulated cell growth and (2) the need for target cell proliferation in order for the nucleic acid sequence encoding the siRNA carried by the vector to be integrated into the target genome.

Another viral candidate useful as an expression vector for transformation of cells is the adenovirus, a double-stranded DNA virus. The adenovirus is infective in a wide range of cell types, including, for example, muscle and endothelial cells. The adenovirus also has been used as an expression vector in muscle cells in vivo. Adenoviruses (Ad) are double-stranded linear DNA viruses with a 36 kb genome. Several features of adenovirus have made them useful as transgene delivery vehicles for therapeutic applications, such as facilitating in vivo gene delivery. Recombinant adenovirus vectors have been shown to be capable of efficient in situ gene transfer to parenchymal cells of various organs, including the lung, brain, pancreas, gallbladder, and liver. This has allowed the use of these vectors in methods for treating inherited genetic diseases, such as cystic fibrosis, where vectors may be delivered to a target organ. In addition, the ability of the adenovirus vector to accomplish in situ tumor transduction has allowed the development of a variety of anticancer gene therapy methods for non-disseminated disease. In these methods, vector containment favors tumor cell-specific transduction. Like the retrovirus, the adenovirus genome is adaptable for use as an expression vector for gene therapy, i.e., by removing the genetic information that controls production of the virus itself. Because the adenovirus functions in an extrachromosomal fashion, the recombinant adenovirus does not have the theoretical problem of insertional mutagenesis.

Several approaches traditionally have been used to generate the recombinant adenoviruses. One approach involves direct ligation of restriction endonuclease fragments containing a nucleic acid sequence of interest to portions of the adenoviral genome. Alternatively, the nucleic acid sequence of interest may be inserted into a defective adenovirus by homologous recombination. The desired recombinants are identified by screening individual plaques generated in a lawn of complementation cells.

Most adenovirus vectors are based on the adenovirus type 5 (Ad5) backbone in which an expression cassette containing the nucleic acid sequence of interest has been introduced in place of the early region 1 (E1) or early region 3 (E3). Viruses in which E1 has been deleted are defective for replication and are propagated in human complementation cells (e.g., 293 or 911 cells), which supply the missing gene E1 and pIX in trans.

Thus, as will be apparent to one of ordinary skill in the art, a variety of suitable viral expression vectors are available for transferring exogenous nucleic acid material into cells. The selection of an appropriate expression vector to express a therapeutic agent for a particular condition amenable to gene silencing therapy or allele-specific silencing therapy, and the optimization of the conditions for insertion of the selected expression vector into the cell, are within the scope of one of ordinary skill in the art without the need for undue experimentation.

In another embodiment, the expression vector is in the form of a plasmid, which is transferred into the target cells by one of a variety of methods: physical, electroporation, scrape loading, microparticle bombardment or by cellular uptake as a chemical complex (e.g., calcium or strontium co-precipitation, complexation with lipid, complexation with ligand)). Several commercial products are available for cationic liposome complexation including Lipofectin™ (Gibco-BRL, Gaithersburg, Md.) and Transfectam™ (ProMega, Madison, Wis.). However, the efficiency of transfection by these methods is highly dependent on the nature of the target cell and accordingly, the conditions for optimal transfection of nucleic acids into cells using the above-mentioned procedures must be optimized. Such optimization is within the scope of one of ordinary skill in the art without the need for undue experimentation.

### VII Methods for Selecting an Antisense Molecule

In diseases caused by an autosomal dominant mutation, one of the alleles of a gene contains a mutation which can have a dominant negative effect. In this case it is desirable to inhibit such mutation present in the affected allele while allowing the unaffected allele of said gene to be expressed. In order to select a suitable antisense molecule which is capable of selectively inhibiting the affected allele, a nucleic acid sample is provided from a patient suffering from a disease caused by an autosomal dominant, dominant-negative mutation. Many such diseases are known. A non-limiting list of examples of such disease include hypertrophic cardiomyopathy, centronuclear myopathy and Marfan syndrome. In principle, any such disease may be treated by the antisense molecule selected according to the invention. The method of the invention requires a nucleic acid, preferably a DNA sample of the patient. Any biological sample from the patient which contains DNA may be used, including but not limited to a blood sample, saliva, cheek mucosa etc.

In certain embodiments RNA may be used. DNA may be isolated from said sample according to methods known in the art. Subsequently, the presence of an autosomal dominant, dominant-negative mutation is determined by determining the nucleic acid sequence of said gene of both alleles present. Subsequently, the alleles are screened for the presence of a heterozygous SNP in the alleles of said gene. In a preferred embodiment a heterozygous SNP is determined by selecting a synonymous SNP, as synonymous SNPs are expected to have the highest minor allele frequencies (MAF). In a step, it is determined which variant of the SNP resides on the mutated allele of the gene.

Then the nucleic acid sequence of the sense strand of said variant of the SNP of said gene is determined using methods known in the art. Subsequently, an isolated antisense molecule is selected which comprises an antisense nucleic acid strand which is substantially complementary to a target region of a transcript encoded by said gene, wherein said antisense nucleic acid strand is at least complementary to said variant of the SNP in said target region.

In a preferred embodiment, said method of selecting an antisense molecule of the invention further comprises a step wherein the selected antisense molecule is tested in a cell to determine the effect on inhibition of expression of said gene. Preferably said method comprises determining the effect of inhibition of expression of the mutant and/or the unmutated allele of said gene. Preferably said method comprises a step of determining a change in allelic imbalance of said gene. Preferably, said step is performed on a cell from the patient.

In a preferred embodiment, the isolated antisense molecule is selected from:
a. a double stranded nucleic acid (dsNA) or a chemically modified version thereof, or
b. an antisense oligonucleotide (ASO).

Preferably, said dsNA is RNA, more preferably a short hairpin (shRNA) or a short interfering RNA (siRNA). Preferably, said dsNA is dsRNA.

In a preferred embodiment of the isolated antisense molecule according to the invention, said antisense nucleic acid strand is of 15-30 nucleotides in length. Preferably, said antisense nucleic acid strand is complementary to at least 7, more preferably 8, 9, 10, 11, 12, 13, 14, 15, 16, 17, 18, 19, 20 or 21 nucleotides in said target region. In some embodiments there may be one or two mismatches between the antisense nucleic acid strand and the target region. Preferably, said antisense nucleic acid strand is complementary to at least 7, more preferably 8, 9, 10, 11 or 12 consecutive nucleotides. Preferably, said target region comprises at least 15, 16, 17, 18 or 19 nucleotides in said target region.

In a preferred embodiment of the isolated double stranded nucleic acid (dsNA) according to the invention, the dsNA comprises a sense nucleic acid strand of 15-30 nucleotides in length; and the sense nucleic acid strand is complementary to said antisense nucleic acid strand and the sense and antisense nucleic acid strands form a duplex region. Preferably, said dsNA is a shRNA containing an antisense nucleic acid strand of RNA of 15 to 30 nucleotides, preferably 19 nucleotides in length having a 5′ end and a 3′ end, wherein the antisense nucleic acid strand is preferably complementary to at least 15 nucleotides of said target region, and wherein preferably the 5′ end of the sense strand of RNA is operably linked to a G nucleotide to form a first segment of RNA, and a sense strand of RNA of 15 to 30 nucleotides in length having a 5′ end and a 3′ end, wherein preferably at least 12 nucleotides of the antisense and sense strands are complementary to each other and preferably form a small interfering RNA (siRNA) duplex under physiological conditions.

Preferably, the antisense strand and the sense strand are operably linked by means of an RNA loop strand to form a hairpin structure comprising a duplex structure and a loop structure. Preferably, the loop structure preferably contains from 4 to 13, more preferably between 4 and 10 nucleotides. In an embodiment, said loop structure contains 4, 5 or 6 nucleotides. Preferably said loop contains the sequence UCAAGAC. The duplex formed by the two strands of RNA may be between 15 and 25 base pairs in length, preferably 19 base pairs in length. The antisense strand preferably is 19 nucleotides in length. The sense strand preferably is 19 nucleotides in length. The shRNA may further contain an overhang region. Such an overhang may be a 3′ overhang region or a 5′ overhang region. The overhang region may be, for example, from 1 to 6 nucleotides in length.

The invention further provides a nucleic acid encoding the isolated antisense molecule of the invention. In a preferred embodiment, said shRNA of the invention is encoded by an oligonucleotide having the following structure: forward 5′-CCGGAA-19 bp sense strand-TCAAGAC-19 bp antisense strand-TTTTTTTG-3′ and/or reverse 5′-AATTCAAAAAAA-19 bp sense strand-GTCTTGA-19 bp antisense strand-TT-3′. Herein the sense strand has the same sequence as the targeted sequence in the mRNA and the antisense strand its reverse complementary sequence that will eventually bind the mRNA and induce its breakdown and/or inhibition.

Preferably, said gene is selected from the KCNQ1, NF1, FBN1, BRCA1, BRCA2, TSC1 and TSC2 gene.

Preferably, said SNP is selected from rs1057128, rs8234 and rs17215465. Preferably, said disease is a cardiac arrhythmia, tuberous sclerosis, breast cancer, neurofibromitosis Type I, Huntington disease, hypertrophic cardiomyopathy, polycystic kidney disease, osteogenesis imperfecta, chondrodysplasia, centronuclear myopathy and Marfan syndrome. Preferably said cardiac arrhythmia is long or short QT syndrome, more preferably of long QT syndrome type 1 or short QT syndrome type 2.

In a preferred embodiment, said antisense molecule is as described herein. Preferably, said gene is the KCNQ1 gene. Preferably, said SNP is selected from rs8234, rs17215465 and rs1057128.

The invention will now be illustrated by the following non-limiting Example.

### EXAMPLE

### Example 1. Allele-Specific Targeting of Common SNPs in KCNQ1

To allow allele-specific downregulation of KCNQ1 by targeting common SNPs, we searched the coding region of KCNQ1 for synonymous SNPs because these were expected to have the highest minor allele frequencies (MAF). Consequently, the highest amount of patients will be heterozygous for these SNPs, which allows allele-specific targeting. We found 2 synonymous SNPs in KCNQ1 with a MAF in the Genome Aggregation Database (gnomAD) population of 1.6% (rs17215465) and 16.6% (rs1057128), which translates to a heterozygosity of 3 and 27%, respectively. We selected rs1057128 in exon 13 for allele-specific targeting, as this SNP would allow to treat 27% of patients independent of the causal mutation. Because both alleles of the SNP can reside on the mutated allele in different patients, we designed shRNAs to target both alleles of rs1057128. We selected shRNAs targeting the A allele with the mismatch at position 10, 13, 15, 16, and 18 and shRNAs targeting the G allele with the mismatch at position 10, 11, 12, 16, and 18.

To test these shRNAs we used two hiPSC-CM lines, Line 1 and Line 2, derived from two brothers with an R243C mutation in exon 5 of KCNQ1. These brothers were 42 and 40 years old when dermal fibroblasts were obtained, they were asymptomatic and had at rest a QTc interval on their ECG of 434 ms (age 36) and 509 ms (age 34), respectively. Both brothers are heterozygous for SNP rs1057128 and carry the A allele on the mutant KCNQ1 allele.

We lentivirally transduced hiPSC-CMs of both lines with the shRNAs targeting the A or G allele of the SNP and compared by allele-specific qRT-PCR expression levels of mutant and wild-type KCNQ1 alleles to expression levels in cells transduced with a scrambled negative control shRNA (shSCR). Targeting the A allele of rs1057128, which resides in these hiPSC lines on the mutant KCNQ1 allele, revealed one shRNA, shA18, which downregulated the mutant KCNQ1 allele without affecting the wild-type allele in both hiPSC lines (FIG. 1A-H). ShA10 also downregulated the mutant allele in Line 1, yet, it was not allele-specific and expression of the wild-type allele was partially lost.

We introduced additional mismatches to improve the allele-specific downregulation. We only introduced the additional mismatches to shA18 since shA10 strongly induced an interferon response (FIG. 5A/B), a possible response described before upon introduction of foreign RNA [20]. None of these modifications improved allele-specific downregulation of the mutant KCNQ1 allele by targeting the A allele of rs1057128 (FIG. 5C-J).

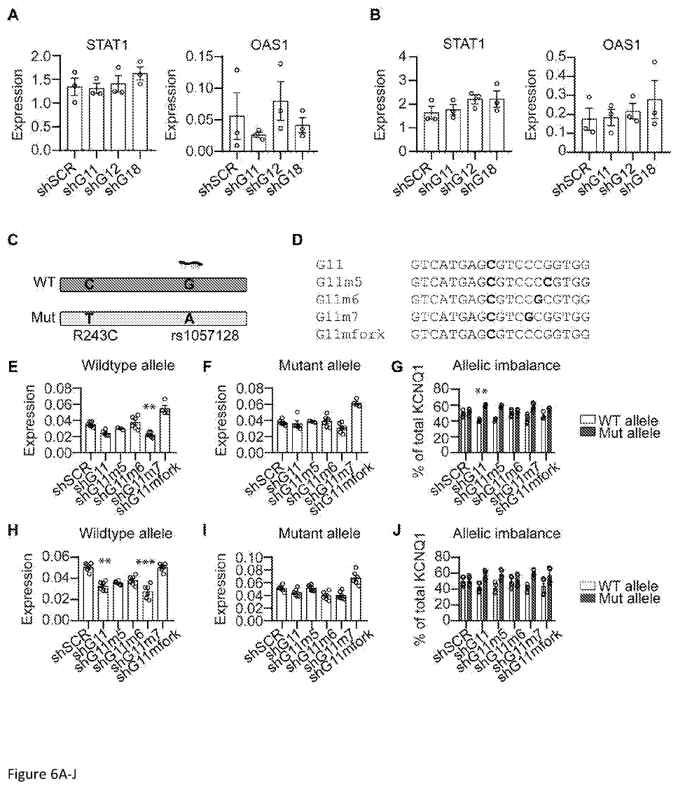

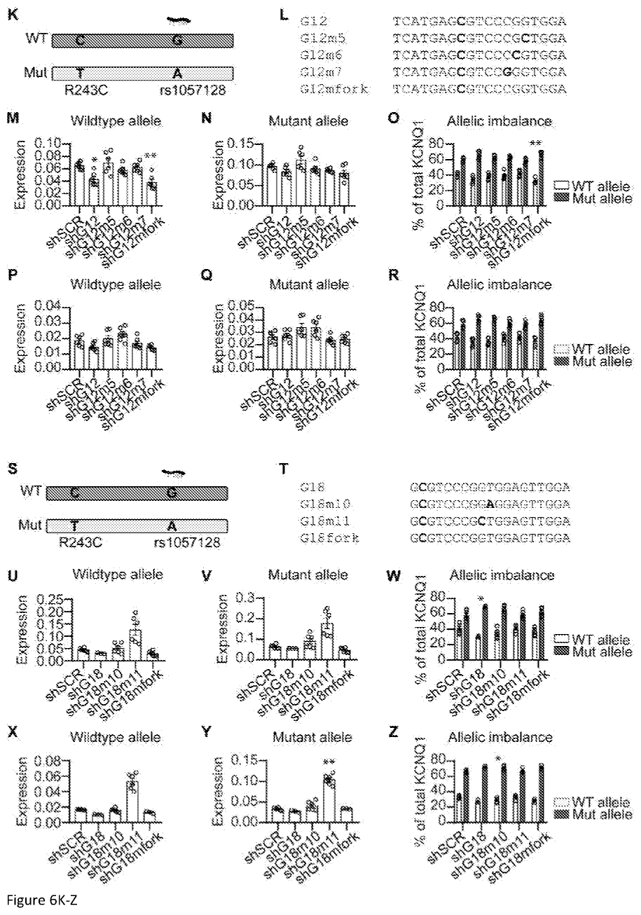

Targeting the G allele of SNP rs1057128, which resides in these hiPSC lines on the wild-type KCNQ1 allele, revealed that 3 shRNAs, i.e. shG11, shG12 and shG18, downregulated in both hiPSC lines the wild-type KCNQ1 allele without affecting the mutant allele (FIG. 1I-P). The two other shRNAs, shG10 and shG16 were not allele-specific and also downregulated expression of the mutant KCNQ1 allele. Because shG11, shG12 and shG18 did not trigger an interferon response (FIG. 6A/B) we introduced additional mismatches and created fork shRNAs for these shRNAs. However, also for these shRNAs targeting the G allele of SNP rs1057128 these modifications did not improve allele-specificity (FIG. 6C-Z).

### Allele-Specific Targeting of SNPs in the 3′UTR of KCNQ1

In our search for common SNPs in the coding region of KCNQ1, we noticed several SNPs with a high MAF in the 3′ UTR of KCNQ1. One of these SNPs is rs8234 with a MAF of 38.5% in the gnomAD population and a corresponding heterozygosity of 47%. This means that targeting this SNP would further increase applicability of allele-specific shRNAs to 47% of the patients. The youngest brother described above (hiPSC Line 2) is also heterozygous for this SNP, and he carries the A allele on the mutant KCNQ1 allele. Therefore, we tested in this line whether this SNP also allowed allele-specific targeting.

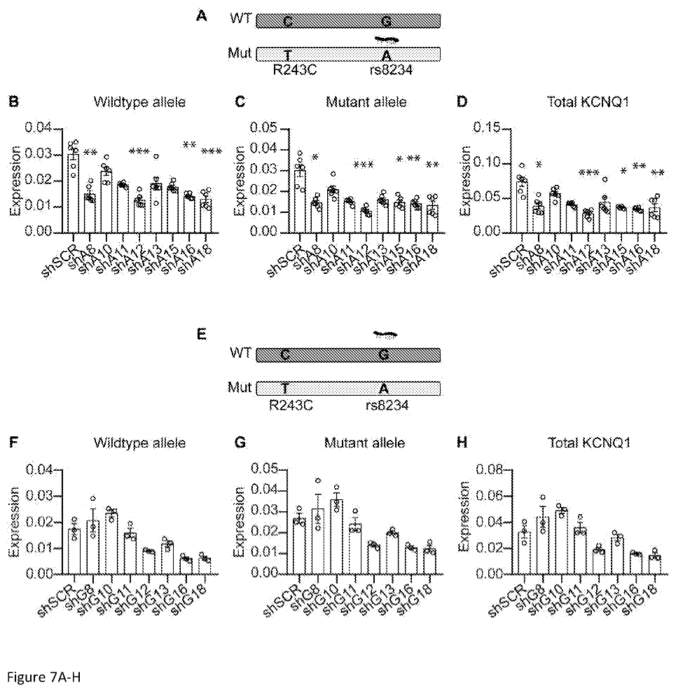

We designed 7 shRNAs to target the A allele of rs8234. Although all were able to downregulate expression of the mutant KCNQ1 allele, none was allele-specific (FIG. 7A-D). We also designed 8 shRNAs to target the G allele of rs8234 on the wild-type KCNQ1 allele. These shRNAs either did not induce a downregulation of any allele or, although not significant, downregulated both alleles (FIG. 7E-H).

Allele-Specific shRNAs Affect the Allelic Balance in hiPSC-CM

Mutations in KCNQ1 often act as dominant-negative mutations [9]. Kv7.1 channels are formed by the assembly of four KCNQ1-encoded Kv7.1 α-subunits, and patients heterozygous for an LQT1-causing mutation combine wild-type and mutant subunits in their channels. The balance between the expression of wild-type and mutant subunits will determine the proportion of mutated subunits in the Kv7.1 channels and thus also the number of fully functional channels formed solely by wild-type subunits. Therefore, we assessed to what extent the allele-specific downregulation by shRNAs affected the balance between wild-type and mutant allele expression in hiPSC-CMs of our two lines.

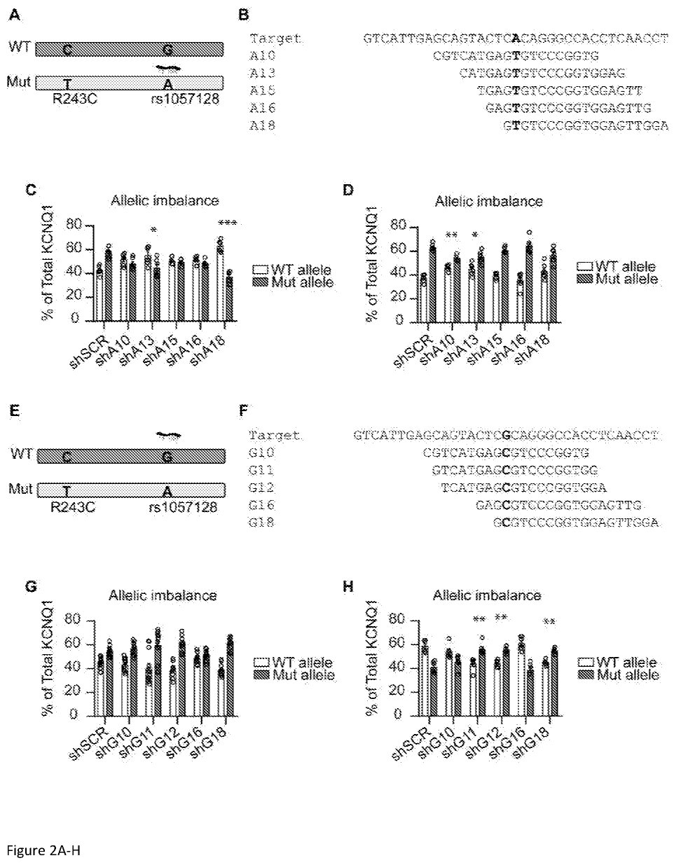

As discussed above, two of our shRNAs targeting the A allele of rs1057128 on the mutant KCNQ1 allele, downregulated the mutant allele in Line 1. Of these two, shA18 was allele-specific while shA10 downregulated both mutant and wild-type alleles. In Line 2, shA18 downregulated the mutant allele significantly. In Line 1, shA18 shifted the allelic balance from wild-type:mutant 43:57% to 63:37% (FIG. 2C). Strikingly, despite that shA10 was not allele-specific in Line 2, this shRNA induced the largest shift in allelic balance in this line as it shifted the wild-type:mutant ratio from 37:63% to 46:54% (FIG. 2D), while shA18, which was allele-specific, shifted the balance to 43:57%. These shifts in allelic imbalance in both lines, together with the induction of an interferon response by shA10 (FIG. 5A/B) made us select shA18 for further functional experiments.

Of our shRNAs targeting the G allele of rs1057128 on the wild-type KCNQ1 allele, 3 shRNAs were able to allele-specifically downregulate the wild-type KCNQ1 allele. In line 1, these shRNAs shifted the allelic balance between wild-type to mutant allele from 45:55% to 41:59% for shG11, 38:62% for shG12, and 39:61% for G18 (FIG. 2G). In line 2, these shRNAs shifted the balance between wild-type to mutant allele from 59:41% to 44:56% for shG11, 44:56% for shG12, and 45:55% for G18 (FIG. 2H). We selected shG11 for further functional experiments based on these shifts in allelic balance combined with the observation that shG11 was the most allele-specific of these three shRNAs (FIG. 1K-P).

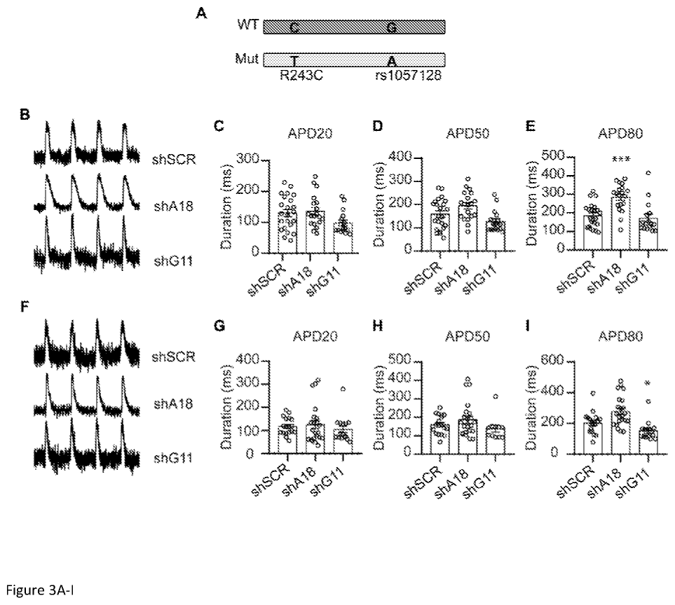

Specific downregulation of the mutant KCNQ1 allele prevents the occurrence of arrhythmic events To evaluate the functional effects of shA18 and shG11 in hiPSC-CMs we used the fluorescence voltage indicator ArcLight A242. ArcLight is a voltage-sensitive fluorescent protein that changes its conformation and thereby its fluorescence levels in prompt response to the voltage dynamics of the action potential. ArcLight-expressing hiPSC-CMs show a reduction in fluorescence intensity in response to depolarization of the cell and an increase in fluorescence in response to the subsequent repolarization [21]. We lentivirally transduced hiPSC-CMs with the shRNAs and a dsRED marker to allow selection of transduced cells. We recorded optical action potentials as fluorescence changes in ArcLight while cells were paced at 1 or 2 Hz to eliminate spontaneous beating and associated effects of beating rate on APD. Because LQT1-causing mutations often act via dominant-negative mechanisms, which reduce the IKs current and compromise repolarization resulting in a prolongation of the APD, we expected that the APD of our LQT1 hiPSC-CM would be shortened by inhibition of the mutant KCNQ1 allele as this would result in more fully wild-type functional tetramers. Surprisingly, we did not find a shortening of the APD at 50 or 80% of repolarization when the mutant KCNQ1 allele was downregulated by shA18, but we actually detected a prolongation of APD80 both at 1 and 2 Hz (FIG. 3E/I). On the contrary, downregulation of the wild-type KCNQ1 allele by shG11 shortened APD80 at 1 Hz, and APD20 and APD50 at 2 Hz stimulation (FIG. 3E/I). These results are in contrast to what we expected from a shift towards less expression of the wild-type KCNQ1 allele, where less expression of the wild-type allele would further decrease the amount of fully functional Kv7.1 channels and therefore decrease the repolarizing IKs. These results might indicate that the loss of functional IKs also affects other ion channels and thereby the electrophysiological characteristics of hiPSC-CMs with a reduced functional IKs.

QTc is often used to diagnose LQTS. However, QTc duration in LQT1 patients is not always directly related to life-threatening arrhythmias and these arrhythmias may also occur in patients with a marginally prolonged QTc, although to a much lesser extent [22]. This means that our shRNAs, although they surprisingly did not affect the APD as expected, still might affect the occurrence of arrhythmic events. Therefore, we evaluated the occurrence of arrhythmic events in shRNA treated hiPSC-CMs. In cells treated with the negative control shRNA, we detected arrhythmic events in 7 and 12% of hiPSC-CMs of Lines 1 and 2, respectively. Downregulation of the mutant KCNQ1 allele by shA18 completely abolished the occurrence of arrhythmic events in Line 1 and reduced them to 9% in Line 2 (FIG. 4, chi-square compared to control 0.234 and 0.622 respectively). This indicates that the reduction of the mutant KCNQ1 allele and the resulting shift in allelic imbalance towards the wild-type KCNQ1 allele, does improve the LQT1 phenotype even though no effect on APD was observed. Further in line with these findings, downregulation of the wild-type KCNQ1 allele by shG11 increased the number of hiPSC-CM with arrhythmic events to 56% in Line 1 and 51% in Line 2 (FIG. 4, chi-square compared to control <0.001 for both lines).

Computer simulations demonstrate the applicability of allele-specific inhibition in an adult human cardiomyocyte model. To investigate the applicability of the allele-specific shRNAs in treating LQT1 patients beyond the effects shown for the R243C mutation present in our hiPSC lines, we performed computer simulations in an adult human cardiomyocyte model. In this model, we first assumed that presence of a single mutant subunit in the tetrameric channel fully abrogates the channel function. Because both shRNAs targeting the A and G allele of rs1057128 downregulate the targeted allele by 40-60%, we investigated the effect of a 60% reduction of mutant channels. In a situation where both mutant and wild-type KCNQ1 alleles are equally expressed and co-assemble randomly, only 1 out of 16 of the IKs channels (6.25%) will consist of only wild-type subunits. Reduction of the mutant KCNQ1 allele by 60% would increase this number to 26%. In a situation where only IKs channels entirely built of wild-type subunits are conductive, this might increase IKs approximately four times. This was indeed the case and was accompanied by a 30% decrease in APD90 in both the epicardial and endocardial simulations and 12% in the mid-myocardial ones.

In a second simulation experiment, we included characteristics of heterotetrameric channels formed by mutant subunits carrying an E160K mutation in KCNQ1 and wild-type subunits. Again, we simulated what would be the result of a 60% reduction in the amount of mutant subunits.

Restoration of IKs through a 60% suppression of the E160K mutant subunits expression is not very effective if IKs channels with 1-4 mutant subunits contribute equally to the heterotetrameric IKs, which was simulated by a 68.4% reduction in IKs conductance and a +7.9 mV shift in the IKs steady-state activation curve. This is because the beneficial effects of the increased fraction of channels with only wild-type subunits is largely counteracted by the reduced total number of channels due to the reduced expression of subunits simulations labelled ‘Suppression 1’. However, if only channels with a single mutant KCNQ1 subunit are conductive and channels with 2-4 mutant subunits are not, 60% suppression of E160K mutant subunits is effective simulations labelled ‘Suppression 2’ because the fraction of channels with only one mutant subunit increases. This assumption seems reasonable for the E160K mutation, considering that the peak amplitude of the heterozygous WT/E160K IKs measured by Vanoye et al. was 31.6±5.9% (mean±SEM, n=34) of the homozygous wild-type control and the fraction of IKs channels with three or four wild-type subunits (so maximally one mutant subunit) accounts for 31% of the channels (25 and 6% respectively) when both alleles are equally expressed.

### Methods

Human iPSC Generation

All studies conform to the declaration of Helsinki and were approved by the Medical Ethics Committee of the Amsterdam UMC, Amsterdam. The skin biopsies were obtained after individual permission using standard informed consent procedures. Dermal fibroblasts were obtained from two brothers of 42 and 40 years of age with a diagnosis of familial LQT1 due to an R243C missense mutation in KCNQ1. Fibroblasts were retrovirally reprogrammed with the transcription factors OCT4, SOX2, and KLF4 with addition of valproic acid as described previously [41]. Human iPSC culture hiPSCs were cultured in mTeSR-1 (StemCell Technologies; 85850) on plates coated with 1:500 diluted growth factor-reduced Matrigel (Corning; FAL356231). Cells were passaged every 4-6 days via dissociation with 0.5 mM EDTA (Invitrogen; 15575-038) and seeded in mTeSR-1 supplemented with 2 μM Thiazovivin (Selleck Chemicals; S1459). Between passages mTeSR-1 medium was replaced every day, except for the first day after passaging.

### Karyotype Analysis

Karyotypes were determined using G-banding chromosome analysis according to standard procedures by the institutional cytogenetic laboratory of the Rambam Medical Center, Haifa.

Cardiac Differentiation of hiPSC

Differentiation towards cardiomyocytes was performed following a previously published protocol with slight adaptations [42]. Differentiation was induced 4 days after passaging by changing to CDM3 medium (RPMI-1640, Gibco 21875; 500 μg/ml human serum albumin, Sigma A9731; 213 g/ml L-ascorbic acid 2-phosphate, Sigma A8960; 1% penicillin/streptomycin) supplemented with 6 μM CHIR99021 (Stemgent; 04-0004-10) for two days, followed by CDM3 supplemented with 2 μM Wnt-C59 (Selleck Chemicals; S7037) for two days. From day 4 to day 10, medium was changed every other day for RPMI/B27 medium (RPMI-1640; 2% B27 supplement minus insulin, Gibco A1895601; 1% penicillin/streptomycin). Spontaneous hiPSC-CM contractions could be identified from day 8 onwards. From day 10 onwards, metabolic cardiomyocyte selection was performed by replacing the hiPSC-CM medium once every week with CDM3 medium without glucose (RPMI-1640 without glucose, Gibco 11879) supplemented with 20 mM sodium-lactate (Sigma-Aldrich; L7022; dissolved in 1 M HEPES-solution) for at least 2 weeks [43]. After selection medium was replaced once a week with CDM3 medium with glucose. hiPSC-CM were either dissociated by TrypLE Express (Gibco; 12604) or TrypLE Select (Gibco; A1217701) with an incubation of 15 minutes and plated on matrigel coated plates or coverslips in RPMI/B27 medium with 2 μM Thiazovivin for shRNA selection experiments and basal characterization or in CDM3 medium containing lentivirus supplemented with 2 μM Thiazovivin for electrophysiology experiments. All experiments were conducted on hiPSC-CMs 40-60 days after start of differentiation and each observation was replicated in 2 to 5 independent experiments with hiPSC-CMs from different differentiations.

### In Vitro Trilineage Differentiation Potential

Trilineage differentiation potential was assessed by induction of endodermal, ectodermal and mesodermal differentiation of hiPSCs using the STEMdiff Trilineage Differentiation Kit (STEMCELL Technologies; 05230) according to the manufacturer's protocol.

### Immunocytochemistry

Cells for immunocytochemistry were plated on 12 mm glass coverslips coated with matrigel. Undifferentiated hiPSCs were cultured in mTeSR-1 for 3 days and differentiated hiPSC-CMs for 1 week in RPMI/B27. Cells were fixed in 4% paraformaldehyde for 15 minutes at room temperature and washed 3 times in PBS. Cells were permeabilized with Triton X-100 in PBS for 8 minutes (0.1% for hiPSC-CMs and 1% for hiPSCs). Unspecific antibody binding was blocked by 20 minutes incubation with 4% goat or 10% horse serum. Primary antibodies (Table 1) were diluted in PBS with 4% goat or 10% horse serum and incubated overnight at 4° C. Cells were washed 3 times in PBST and incubated for 1 hour at room temperature in the dark with 1:250 diluted secondary antibodies (Table 1) in PBS with 4% goat or 10% horse serum. Cells were washed 3 times in PBST. Nuclei were counterstained with DAPI (1:5000) for 5 minutes and mounted in Mowiol (Sigma, 81381).

### Plasmid Generation

For shRNA expression, we used the pLKO.1 backbone either with puromycin as selection marker for shRNA selection (pLKO.1-puro; Addgene 8453) or with dsRED as fluorescent marker for electrophysiology [44]. Cloning of shRNA sequences was similar in the pLKO.1-puro and pLKO.1-dsRED plasmids. Therefore, we designed the following oligonucleotides: forward 5′-CCGGAA-19 bp sense strand-TCAAGAC-19 bp antisense strand-TTTTTTTG-3′ and reverse 5′-AATTCAAAAAAA-19 bp sense strand-GTCTTGA-19 bp antisense strand-TT-3′. The sense strand is exactly the mRNA targeting sequence and the antisense strand its reverse complementary sequence that will eventually bind the mRNA. We annealed 1 nMol of these oligonucleotides and cloned them into Agel and EcoRI restriction sites in the pLKO.1 plasmids. Exact shRNA sequences are detailed in Table 2. A shRNA with a scrambled sequence (shSCR) was used as a negative control shRNA [44]. PLV-CAG-ArcLight was previously described [45]. All plasmid sequences were verified by Sanger sequencing and occurrence of mutations excluded.

### Virus Production

To produce third-generation lentivirus of pLKO.1-puro, pLKO.1-dsRED and pLV-CAG-ArcLight based constructs we co-transfected 4.106 HEK293T cells with 4 μg of the expression plasmid, 2.7 μg pMDLg/pRRE, 1 μg pRSV-Rev and 1.4 μg p VSVG using Genejammer (Agilent; 204130) according to the manufacturer's protocol. The next day, medium was replaced with CDM3 medium. This medium containing the produced lentivirus was collected after 24 hours and either used directly for hiPSC-CM transduction or the amount of transducing units (TU) was first determined. The amount of TU was determined by transducing 250000 HEK293T cells with series of 50/100/200/500/1000 μl of medium with virus of the pLKO.1-dsRED plasmid. Three days after transduction, the cells were trypsinized and analyzed by FACS for the dsRED positive population. The condition with 10-20% positive cells was used to calculate the amount of TU assuming 1 viral copy per cell. The amount of TU for an experimental virus and its corresponding control were determined in the same FACS experiment.

hiPSC-CM Infection

For shRNA selection experiments, hiPSC-CMs were dissociated and replated in 6 well plates 2 to 4 days before lentiviral transduction to ensure homogenous cell populations between conditions. For these selection experiments, 2 ml/well of medium with viruses containing the puromycin resistance cassette were freshly added to the hiPSC-CMs. Medium was refreshed the day after transduction. Five days after transduction, puromycin selection started with 8 μg/ml puromycin for 72 hours after which the cells were harvested for RNA experiments.

For electrophysiology experiments, cells were dissociated and resuspended in 1.5 ml medium containing ArcLight-encoding lentivirus (TU not determined) and 30000 TU of shRNA-encoding lentivirus and then plated in 35 mm optical plates (CELLview; 627860). Starting from day two onwards, medium was refreshed with CDM3 medium every other day. Cells were measured 6-8 days after plating and infection.

### RNA Isolation

Total RNA was isolated using 1 ml TriReagent (Sigma Aldrich; T9424). TriReagent was added directly to live cells growing on a dish. Total RNA isolation was performed according to the manufacturer's protocol.

qRT-PCR

To detect mRNA levels 250 ng to 1 μg RNA was DNAse treated with DNAseI amplification grade (Invitrogen; 18068015) and reverse transcribed using Superscript II reverse transcriptase (Invitrogen; 18064014) with oligo-dT and random hexamer primers according to the manufacturer's protocol. cDNA was diluted 5 times and 2 μl was used as input for qPCR. qPCR was performed using 1 μM primers (Table 3) and LightCycler 480 SYBR Green master 1 (Roche; 04887352001) on a LightCycler 480 system II (Roche) using the following cycling program: 5 minutes pre-incubation at 95° C.; 40 cycles of 10 seconds denaturation at 95° C., 20 seconds annealing (temperatures in Table 3), and 20 seconds elongation at 72° C. Data were analyzed using LinRegPCR quantitative PCR analysis software [46] and the starting concentration of transcripts estimated by this software was corrected for the geometric mean of three reference genes: HPRT, GAPDH and TBP. For allele-specific qRT-PCRs, allele specificity was obtained by allele-specific forward primers with the R243C mutation being the very last nucleotide on the 3′-end of the primer, which were combined with a common reverse primer. Allelic imbalance was assessed by comparing the expression of wild-type and mutant KCNQ1 mRNA as percentages of the total KCNQ1 expression, where the total KCNQ1 is the sum of expression of both alleles.

### ArcLight Measurements

For ArcLight measurements, fluorescence was measured with a Leica TCS SP8 SMD mounted on a Leica DMI6000 inverted confocal microscope with a 40× oil inversion objective. ArcLight was excited with a 488 nm white light laser (WLL) pulsed with pulse picker and light collected with a 2HyD detector. For action potential recordings, fluorescence was recorded in xt line-scan mode with 512 pixels per frame at 1 frame per 2 ms. Only dsRed positive cells were measured in Tyrode's solution containing (in mM): NaCl 140; KCl 5.4; CaCl2 1.8; MgCl2 1; HEPES 10; and glucose 10 (pH 7.4; NaOH), while incubated at 37° C. by an incubator enclosing the microscope. Cells were paced with field stimulation via carbon electrodes (P0003-7, EHT Technologies GmbH) at 1 or 2 Hz with a custom made stimulator.

Xt recordings of fluorescence were converted into comma-separated values files with ImageJ for further analysis. Recordings were further analyzed by custom made Matlab software [45]. First, the fluorescence axis of the ArcLight optical signals was inverted. APD20, APD50 and APD80 were calculated as the median time interval of 5-12 action potentials required to reach 20, 50 and 80% of repolarization starting from 50% maximal upstroke height. For the comparison of arrhythmic events, each cell with arrhythmic morphologies (e.g. double beats, EADs) either at baseline while spontaneous beating or when stimulated was counted as a cell with events.

### Computer Simulations

Functional effects of changes in IKs were assessed by computer simulations using the epicardial, midmyocardial, and endocardial versions of the human ventricular cell model by Ten Tusscher et al. [47], as updated by Ten Tusscher and Panfilov [48]. The numerical reconstruction was carried out on an Intel i7 CPU based workstation using Intel Visual Fortran and employing a simple and efficient Euler-type integration scheme with a time step of 5 μs. Simulations were run for a sufficiently long time to achieve steady-state conditions.

### Statistics

Data obtained from hiPSC-CMs are a combination of 2 to 5 independent experiments on cells from independent differentiations, with at least two biological replicates per experiment. Data of these independent experiments are combined using Factor Correction [49], where the control condition was used as a reference to calculate the correction factor by which all the data points of that experiment were corrected. As a consequence, data shown for continuous variables are a mean±SEM of 6-15 biological replicates derived from 2-5 differentiations. For categorical data the percentage of cells in all groups is depicted per condition. Continuous variables were analyzed with GraphPad Prism Software version 8 and the different groups compared by a Kruskal-Wallis test in combination with Dunn's post-hoc test. For comparisons of the allelic imbalance, the ratio between wild-type and mutant KCNQ1 expression was used a continuous variable. Categorical variables were compared to the shSCR negative control by chi-square tests. P-values smaller than 0.05 were considered significant.

TABLE 1

| Antibody | Supplier | Species | Dilution | Cat. number |
|---|---|---|---|---|
| OCT-4 | STEMCELL | Mouse | 1:200 | 60093 |
| | Technologies | | | |
| SSEA-4 | STEMCELL | Mouse | 1:100 | 60062 |
| | Technologies | | | |
| Tra1-60 | STEMCELL | Mouse | 1:100 | 60064 |
| | Technologies | | | |
| Nanog | Peprotech | Rabbit | 1:200 | 500-P236 |
| α-Actinin | Abcam | Rabbit | 1:500 | 68167 |
| cTnI | HyTest | Goat | 1:400 | 4T21/2 |
| Nestin | STEMCELL | Mouse | 1:1000 | 60091 |
| | Technologies | | | |
| AFP | ThermoFisher | Rabbit | 1:100 | PA5-16658 |
| Desmin | ThermoFisher | Rabbit | 1:100 | PA5-16705 |
| Alexa fluor | Invitrogen | Goat | 1:250 ICC | A32731 |
| 488 Anti- | | | | |
| rabbit | | | | |
| Alexa fluor | Invitrogen | Goat | 1:250 ICC | A31620 |
| 488 Anti- | | | | |
| mouse | | | | |
| Alexa fluor | Invitrogen | Donkey | 1:250 ICC | A10037 |
| 488 Anti- | | | | |
| rabbit | | | | |
| Alexa fluor | Invitrogen | Donkey | 1:250 ICC | A32814 |
| 647 Anti- | | | | |
| goat | | | | |

TABLE 2

| Targeted | Position | | Oligonucleotides for cloning of shRNAs |
|---|---|---|---|
| SNP | mismatch | SEQ ID NO | (upper forward and lower reverse oligonucleotide) |
| rs1057128 | A18 | SEQ ID NO: 5 | ccggaaCACAGGGCCACCTCAACCTtcaagacAGGTTGAGGTGGCCCTGTGtttttttg |
| | | SEQ ID NO: 6 | aattcaaaaaaaCACAGGGCCACCTCAACCTgtcttgaAGGTTGAGGTGGCCCTGTGtt |
| rs1057128 | A10 | SEQ ID NO: 7 | ccggaaGCAGTACTCACAGGGCCACtcaagacGTGGCCCTGTGAGTACTGCtttttttg |
| | | SEQ ID NO: 8 | aattcaaaaaaaGCAGTACTCACAGGGCCACgtcttgaGTGGCCCTGTGAGTACTGCtt |
| rs1057128 | A13 | SEQ ID NO: 9 | ccggaaGTACTCACAGGGCCACCTCtcaagacGAGGTGGCCCTGTGAGTACtttttttg |
| | | SEQ ID NO: 10 | aattcaaaaaaaGTACTCACAGGGCCACCTCgtcttgaGAGGTGGCCCTGTGAGTACtt |
| rs1057128 | A15 | SEQ ID NO: 11 | ccggaaACTCACAGGGCCACCTCAAtcaagacTTGAGGTGGCCCTGTGAGTtttttttg |
| | | SEQ ID NO: 12 | aattcaaaaaaaACTCACAGGGCCACCTCAAgtcttgaTTGAGGTGGCCCTGTGAGTtt |
| rs1057128 | A16 | SEQ ID NO: 13 | ccggaaCTCACAGGGCCACCTCAACtcaagacGTTGAGGTGGCCCTGTGAGtttttttg |
| | | SEQ ID NO: 14 | aattcaaaaaaaCTCACAGGGCCACCTCAACgtcttgaGTTGAGGTGGCCCTGTGAGtt |
| rs1057128 | G18 | SEQ ID NO: 15 | ccggaaCGCAGGGCCACCTCAACCTtcaagacAGGTTGAGGTGGCCCTGCGtttttttg |
| | | SEQ ID NO: 16 | aattcaaaaaaaCGCAGGGCCACCTCAACCTgtcttgaAGGTTGAGGTGGCCCTGCGtt |
| rs1057128 | G10 | SEQ ID NO: 17 | ccggaaGCAGTACTCGCAGGGCCACtcaagacGTGGCCCTGCGAGTACTGCtttttttg |
| | | SEQ ID NO: 18 | aattcaaaaaaaGCAGTACTCGCAGGGCCACgtcttgaGTGGCCCTGCGAGTACTGCtt |
| rs1057128 | G11 | SEQ ID NO: 19 | ccggaaCAGTACTCGCAGGGCCACCtcaagacGGTGGCCCTGCGAGTACTGtttttttg |
| | | SEQ ID NO: 20 | aattcaaaaaaaCAGTACTCGCAGGGCCACCgtcttgaGGTGGCCCTGCGAGTACTGtt |
| rs1057128 | G12 | SEQ ID NO: 21 | ccggaaAGTACTCGCAGGGCCACCTtcaagacAGGTGGCCCTGCGAGTACTtttttttg |
| | | SEQ ID NO: 22 | aattcaaaaaaaAGTACTCGCAGGGCCACCTgtcttgaAGGTGGCCCTGCGAGTACTtt |
| rs1057128 | G16 | SEQ ID NO: 23 | ccggaaCTCGCAGGGCCACCTCAACtcaagacGTTGAGGTGGCCCTGCGAGtttttttg |
| | | SEQ ID NO: 24 | aattcaaaaaaaCTCGCAGGGCCACCTCAACgtcttgaGTTGAGGTGGCCCTGCGAGtt |
| rs1057128 | G11m5 | SEQ ID NO: 25 | ccggaaCAGTACTCGCAGGGGCACCtcaagacGGTGCCCCTGCGAGTACTGtttttttg |
| | | SEQ ID NO: 26 | aattcaaaaaaaCAGTACTCGCAGGGGCACCgtcttgaGGTGCCCCTGCGAGTACTGtt |
| rs1057128 | G11m6 | SEQ ID NO: 27 | ccggaaCAGTACTCGCAGGCCCACCtcaagacGGTGGGCCTGCGAGTACTGtttttttg |
| | | SEQ ID NO: 28 | aattcaaaaaaaCAGTACTCGCAGGCCCACCgtcttgaGGTGGGCCTGCGAGTACTGtt |
| rs1057128 | G11m7 | SEQ ID NO: 29 | ccggaaCAGTACTCGCAGCGCCACCtcaagacGGTGGCGCTGCGAGTACTGtttttttg |
| | | SEQ ID NO: 30 | aattcaaaaaaaCAGTACTCGCAGCGCCACCgtcttgaGGTGGCGCTGCGAGTACTGtt |
| rs1057128 | G11fork | SEQ ID NO: 31 | ccggaaCAGTACTCGCAGGGCCATAtcaagacGGTGGCCCTGCGAGTACTGtttttttg |
| | | SEQ ID NO: 32 | aattcaaaaaaaCAGTACTCGCAGGGCCACCgtcttgaTATGGCCCTGCGAGTACTGtt |
| rs1057128 | G12m5 | SEQ ID NO: 33 | ccggaaAGTACTCGCAGGGCGACCTtcaagacAGGTCGCCCTGCGAGTACTtttttttg |
| | | SEQ ID NO: 34 | aattcaaaaaaaAGTACTCGCAGGGCGACCTgtcttgaAGGTCGCCCTGCGAGTACTtt |
| rs1057128 | G12m6 | SEQ ID NO: 35 | ccggaaAGTACTCGCAGGGGCACCTtcaagacAGGTGCCCCTGCGAGTACTtttttttg |
| | | SEQ ID NO: 36 | aattcaaaaaaaAGTACTCGCAGGGGCACCTgtcttgaAGGTGCCCCTGCGAGTACTtt |
| rs1057128 | G12m7 | SEQ ID NO: 37 | ccggaaAGTACTCGCAGGCCCACCTtcaagacAGGTGGGCCTGCGAGTACTtttttttg |
| | | SEQ ID NO: 38 | aattcaaaaaaaAGTACTCGCAGGCCCACCTgtcttgaAGGTGGGCCTGCGAGTACTtt |
| rs1057128 | G12fork | SEQ ID NO: 39 | ccggaaAGTACTCGCAGGGCCACTAtcaagacAGGTGGCCCTGCGAGTACTtttttttg |
| | | SEQ ID NO: 40 | aattcaaaaaaaAGTACTCGCAGGGCCACCTgtcttgaTAGTGGCCCTGCGAGTACTtt |
| rs1057128 | G18m10 | SEQ ID NO: 41 | ccggaaCGCAGGGCCTCCTCAACCTtcaagacAGGTTGAGGAGGCCCTGCGtttttttg |
| | | SEQ ID NO: 42 | aattcaaaaaaaCGCAGGGCCTCCTCAACCTgtcttgaAGGTTGAGGAGGCCCTGCGtt |
| rs1057128 | G18m11 | SEQ ID NO: 43 | ccggaaCGCAGGGCGACCTCAACCTtcaagacAGGTTGAGGTCGCCCTGCGtttttttg |
| | | SEQ ID NO: 44 | aattcaaaaaaaCGCAGGGCGACCTCAACCTgtcttgaAGGTTGAGGTCGCCCTGCGtt |
| rs1057128 | G18fork | SEQ ID NO: 45 | ccggaaCGCAGGGCCACCTCAACTAtcaagacAGGTTGAGGTGGCCCTGCGtttttttg |
| | | SEQ ID NO: 46 | aattcaaaaaaaCGCAGGGCCACCTCAACCTgtcttgaTAGTTGAGGTGGCCCTGCGtt |
| rs1057128 | A18m11 | SEQ ID NO: 47 | ccggaaCACAGGGCGACCTCAACCTtcaagacAGGTTGAGGTCGCCCTGTGtttttttg |
| | | SEQ ID NO: 48 | aattcaaaaaaaCACAGGGCGACCTCAACCTgtcttgaAGGTTGAGGTCGCCCTGTGtt |
| rs1057128 | A18m10 | SEQ ID NO: 49 | ccggaaCACAGGGCCTCCTCAACCTtcaagacAGGTTGAGGAGGCCCTGTGtttttttg |
| | | SEQ ID NO: 50 | aattcaaaaaaaCACAGGGCCTCCTCAACCTgtcttgaAGGTTGAGGAGGCCCTGTGtt |
| rs1057128 | A18fork | SEQ ID NO: 51 | ccggaaCACAGGGCCACCTCAACTAtcaagacAGGTTGAGGTGGCCCTGTGtttttttg |
| | | SEQ ID NO: 52 | aattcaaaaaaaCACAGGGCCACCTCAACCTgtcttgaTAGTTGAGGTGGCCCTGTGtt |
| rs8234 | A8 | SEQ ID NO: 53 | ccggaaCTGGGCATTACATCGCATAtcaagacTATGCGATGTAATGCCCAGtttttttg |
| | | SEQ ID NO: 54 | aattcaaaaaaaCTGGGCATTACATCGCATAgtcttgaTATGCGATGTAATGCCCAGtt |
| rs8234 | A10 | SEQ ID NO: 55 | ccggaaGGGCATTACATCGCATAGAtcaagacTCTATGCGATGTAATGCCCtttttttg |
| | | SEQ ID NO: 56 | aattcaaaaaaaGGGCATTACATCGCATAGAgtcttgaTCTATGCGATGTAATGCCCtt |
| rs8234 | A11 | SEQ ID NO: 57 | ccggaaGGCATTACATCGCATAGAAtcaagacTTCTATGCGATGTAATGCCtttttttg |
| | | SEQ ID NO: 58 | aattcaaaaaaaGGCATTACATCGCATAGAAgtcttgaTTCTATGCGATGTAATGCCtt |
| rs8234 | A12 | SEQ ID NO: 59 | ccggaaGCATTACATCGCATAGAAAtcaagacTTTCTATGCGATGTAATGCtttttttg |
| | | SEQ ID NO: 60 | aattcaaaaaaaGCATTACATCGCATAGAAAgtcttgaTTTCTATGCGATGTAATGCtt |
| rs8234 | A13 | SEQ ID NO: 61 | ccggaaCATTACATCGCATAGAAATtcaagacATTTCTATGCGATGTAATGtttttttg |
| | | SEQ ID NO: 62 | aattcaaaaaaaCATTACATCGCATAGAAATgtcttgaATTTCTATGCGATGTAATGtt |
| rs8234 | A15 | SEQ ID NO: 63 | ccggaaTTACATCGCATAGAAATCAtcaagacTGATTTCTATGCGATGTAAtttttttg |
| | | SEQ ID NO: 64 | aattcaaaaaaaTTACATCGCATAGAAATCAgtcttgaTGATTTCTATGCGATGTAAtt |
| rs8234 | A16 | SEQ ID NO: 65 | ccggaaTACATCGCATAGAAATCAAtcaagacTTGATTTCTATGCGATGTAtttttttg |
| | | SEQ ID NO: 66 | aattcaaaaaaaTACATCGCATAGAAATCAAgtcttgaTTGATTTCTATGCGATGTAtt |
| rs8234 | A18 | SEQ ID NO: 67 | ccggaaCATCGCATAGAAATCAATAtcaagacTATTGATTTCTATGCGATGtttttttg |
| | | SEQ ID NO: 68 | aattcaaaaaaaCATCGCATAGAAATCAATAgtcttgaTATTGATTTCTATGCGATGtt |
| rs8234 | G8 | SEQ ID NO: 69 | ccggaaCTGGGCATTACGTCGCATAtcaagacTATGCGACGTAATGCCCAGtttttttg |
| | | SEQ ID NO: 70 | aattcaaaaaaaCTGGGCATTACGTCGCATAgtcttgaTATGCGACGTAATGCCCAGtt |
| rs8234 | G10 | SEQ ID NO: 71 | ccggaaGGGCATTACGTCGCATAGAtcaagacTCTATGCGACGTAATGCCCtttttttg |
| | | SEQ ID NO: 72 | aattcaaaaaaaGGGCATTACGTCGCATAGAgtcttgaTCTATGCGACGTAATGCCCtt |
| rs8234 | G11 | SEQ ID NO: 73 | ccggaaGGCATTACGTCGCATAGAAtcaagacTTCTATGCGACGTAATGCCtttttttg |
| | | SEQ ID NO: 74 | aattcaaaaaaaGGCATTACGTCGCATAGAAgtcttgaTTCTATGCGACGTAATGCCtt |
| rs8234 | G12 | SEQ ID NO: 75 | ccggaaGCATTACGTCGCATAGAAAtcaagacTTTCTATGCGACGTAATGCtttttttg |
| | | SEQ ID NO: 76 | aattcaaaaaaaGCATTACGTCGCATAGAAAgtcttgaTTTCTATGCGACGTAATGCtt |
| rs8234 | G13 | SEQ ID NO: 77 | ccggaaCATTACGTCGCATAGAAATtcaagacATTTCTATGCGACGTAATGtttttttg |
| | | SEQ ID NO: 78 | aattcaaaaaaaCATTACGTCGCATAGAAATgtcttgaATTTCTATGCGACGTAATGtt |
| rs8234 | G16 | SEQ ID NO: 79 | ccggaaTACGTCGCATAGAAATCAAtcaagacTTGATTTCTATGCGACGTAtttttttg |
| | | SEQ ID NO: 80 | aattcaaaaaaaTACGTCGCATAGAAATCAAgtcttgaTTGATTTCTATGCGACGTAtt |
| rs8234 | G18 | SEQ ID NO: 81 | ccggaaCGTCGCATAGAAATCAATAtcaagacTATTGATTTCTATGCGACGtttttttg |
| | | SEQ ID NO: 82 | aattcaaaaaaaCGTCGCATAGAAATCAATAgtcttgaTATTGATTTCTATGCGACGtt |

TABLE 3

| | | | Annealing |
|---|---|---|---|
| Gene | Forward | Reverse | temperature |
| KCNQ1_wt | SEQ ID NO: 121 | SEQ ID NO: 122 | 66 |
| KCNQ1_mut | SEQ ID NO: 123 | SEQ ID NO: 124 | 66 |
| KCNQ1_total | SEQ ID NO: 125 | SEQ ID NO: 126 | 66 |
| STAT1 | SEQ ID NO: 127 | SEQ ID NO: 128 | 60 |
| OAS1 | SEQ ID NO: 129 | SEQ ID NO: 130 | 60 |
| GAPDH | SEQ ID NO: 131 | SEQ ID NO: 132 | 60 |
| HPRT | SEQ ID NO: 133 | SEQ ID NO: 134 | 60 |
| TBP | SEQ ID NO: 135 | SEQ ID NO: 136 | 60 |
| NANOG | SEQ ID NO: 137 | SEQ ID NO: 138 | 60 |
| OCT4 | SEQ ID NO: 139 | SEQ ID NO: 140 | 60 |
| OCT4_viral | SEQ ID NO: 141 | SEQ ID NO: 142 | 63 |
| SOX2 | SEQ ID NO: 143 | SEQ ID NO: 144 | 63 |
| SOX2_viral | SEQ ID NO: 145 | SEQ ID NO: 146 | 63 |
| MLC2V | SEQ ID NO: 147 | SEQ ID NO: 148 | 63 |
| NKX2.5 | SEQ ID NO: 149 | SEQ ID NO: 150 | 63 |
| KLF4_viral | SEQ ID NO: 151 | SEQ ID NO: 152 | 63 |
| MYH6 | SEQ ID NO: 153 | SEQ ID NO: 154 | 60 |
| MYH7 | SEQ ID NO: 155 | SEQ ID NO: 156 | 60 |

### REFERENCES

1. Schwartz, P. J.; Stramba-Badiale, M.; Crotti, L.; Pedrazzini, M.; Besana, A.; Bosi, G.; Gabbarini, F.; Goulene, K.; Insolia, R.; Mannarino, S.; et al. Prevalence of the congenital long-QT syndrome. Circulation 2009, 120, 1761-1767, doi: 10.1161/circulationaha.109.863209.
2. Goldenberg, I.; Zareba, W.; Moss, A. J. Long QT Syndrome. Curr Probl Cardiol 2008, 33, 629-694, doi: 10.1016/j.cpcardiol.2008.07.002.
3. Mizusawa, Y.; Horie, M.; Wilde, A. A. Genetic and clinical advances in congenital long QT syndrome. Circ J 2014, 78, 2827-2833, doi: 10.1253/circj.cj-14-0905.
4. Wang, Q.; Curran, M. E.; Splawski, I.; Burn, T. C.; Millholland, J. M.; VanRaay, T. J.; Shen, J.; Timothy, K. W.; Vincent, G. M.; de Jager, T.; et al. Positional cloning of a novel potassium channel gene: KVLQT1 mutations cause cardiac arrhythmias. Nat Genet 1996, 12, 17-23, doi: 10.1038/ng0196-17.
5. Goldenberg, I.; Horr, S.; Moss, A. J.; Lopes, C. M.; Barsheshet, A.; McNitt, S.; Zareba, W.; Andrews, M. L.; Robinson, J. L.; Locati, E. H.; et al. Risk for life-threatening cardiac events in patients with genotype-confirmed long-QT syndrome and normal-range corrected QT intervals. J Am Coll Cardiol 2011, 57, 51-59, doi: 10.1016/j.jacc.2010.07.038.
6. Priori, S. G.; Blomström-Lundqvist, C.; Mazzanti, A.; Blom, N.; Borggrefe, M.; Camm, J.; Elliott, P. M.; Fitzsimons, D.; Hatala, R.; Hindricks, G.; et al. 2015 ESC Guidelines for the management of patients with ventricular arrhythmias and the prevention of sudden cardiac death: The Task Force for the Management of Patients with Ventricular Arrhythmias and the Prevention of Sudden Cardiac Death of the European Society of Cardiology (ESC). Endorsed by: Association for European Paediatric and Congenital Cardiology (AEPC). Eur Heart J 2015, 36, 2793-2867, doi: 10.1093/eurheartj/ehv316.
7. Priori, S. G.; Wilde, A. A.; Horie, M.; Cho, Y.; Behr, E. R.; Berul, C.; Blom, N.; Brugada, J.; Chiang, C. E.; Huikuri, H.; et al. HRS/EHRA/APHRS expert consensus statement on the diagnosis and management of patients with inherited primary arrhythmia syndromes: document endorsed by HRS, EHRA, and APHRS in May 2013 and by ACCF, AHA, PACES, and AEPC in June 2013. Heart Rhythm 2013, 10, 1932-1963, doi: 10.1016/j.hrthm.2013.05.014.
8. Shalaby, F. Y.; Levesque, P. C.; Yang, W. P.; Little, W. A.; Conder, M. L.; Jenkins-West, T.; Blanar, M. A. Dominant-negative KvLQT1 mutations underlie the LQT1 form of long QT syndrome. Circulation 1997, 96, 1733-1736, doi: 10.1161/01.cir.96.6.1733.
9. Huang, H.; Kuenze, G.; Smith, J. A.; Taylor, K. C.; Duran, A. M.; Hadziselimovic, A.; Meiler, J.; Vanoye, C. G.; George, A. L., Jr.; Sanders, C. R. Mechanisms of KCNQ1 channel dysfunction in long QT syndrome involving voltage sensor domain mutations. Sci Adv 2018, 4, eaar2631, doi: 10.1126/sciadv.aar2631.
10. Thomas, D.; Wimmer, A. B.; Karle, C. A.; Licka, M.; Alter, M.; Khalil, M.; Ulmer, H. E.; Kathöfer, S.; Kiehn, J.; Katus, H. A.; et al. Dominant-negative I (Ks) suppression by KCNQ1-deltaF339 potassium channels linked to Romano-Ward syndrome. Cardiovasc Res 2005, 67, 487-497, doi: 10.1016/j.cardiores.2005.05.003.
11. Amin, A. S.; Giudicessi, J. R.; Tijsen, A. J.; Spanjaart, A. M.; Reckman, Y. J.; Klemens, C. A.; Tanck, M. W.; Kapplinger, J. D.; Hofman, N.; Sinner, M. F.; et al. Variants in the 3′ untranslated region of the KCNQ1-encoded Kv7.1 potassium channel modify disease severity in patients with type 1 long QT syndrome in an allele-specific manner. Eur Heart J 2012, 33, 714-723, doi: 10.1093/eurheartj/ehr473.
12. Dainis, A.; Zaleta-Rivera, K.; Ribeiro, A.; Chang, A. C. H.; Shang, C.; Lan, F.; Burridge, P. W.; Liu, W. R.; Wu, J. C.; Chang, A. C. Y.; et al. Silencing of MYH7 ameliorates disease phenotypes in human iPSC-cardiomyocytes. Physiol Genomics 2020, 52, 293-303, doi: 10.1152/physiolgenomics.00021.2020.
13. Zaleta-Rivera, K.; Dainis, A.; Ribeiro, A. J. S.; Cordero, P.; Rubio, G.; Shang, C.; Liu, J.; Finsterbach, T.; Parikh, V. N.; Sutton, S.; et al. Allele-Specific Silencing Ameliorates Restrictive Cardiomyopathy Attributable to a Human Myosin Regulatory Light Chain Mutation. Circulation 2019, 140, 765-778, doi: 10.1161/circulationaha.118.036965.
14. Matsa, E.; Dixon, J. E.; Medway, C.; Georgiou, O.; Patel, M. J.; Morgan, K.; Kemp, P. J.; Staniforth, A.; Mellor, I.; Denning, C. Allele-specific RNA interference rescues the long-QT syndrome phenotype in human-induced pluripotency stem cell cardiomyocytes. Eur Heart J 2014, 35, 1078-1087, doi: 10.1093/eurheartj/eht067.
15. Bongianino, R.; Denegri, M.; Mazzanti, A.; Lodola, F.; Vollero, A.; Boncompagni, S.; Fasciano, S.; Rizzo, G.; Mangione, D.; Barbaro, S.; et al. Allele-Specific Silencing of Mutant mRNA Rescues Ultrastructural and Arrhythmic Phenotype in Mice Carriers of the R4496C Mutation in the Ryanodine Receptor Gene (RYR2). Circ Res 2017, 121, 525-536, doi: 10.1161/circresaha.117.310882.
16. Lu, X.; Yang, X.; Huang, X.; Huang, C.; Sun, H. H.; Jin, L.; Xu, W.; Mao, H.; Guo, J.; Zhou, J.; et al. RNA interference targeting E637K mutation rescues hERG channel currents and restores its kinetic properties. Heart Rhythm 2013, 10, 128-136, doi: 10.1016/j.hrthm.2012.09.124.
17. Dotzler, S. M.; Kim, C. S.; Gendron, W. A. C.; Zhou, W.; Ye, D.; Bos, J. M.; Tester, D. J.; Barry, M. A.; Ackerman, M. J. Suppression-Replacement KCNQ1 Gene Therapy for Type 1 Long QT Syndrome. Circulation 2021, 143, 1411-1425, doi: 10.1161/circulationaha.120.051836.
18. Huang, H.; Qiao, R.; Zhao, D.; Zhang, T.; Li, Y.; Yi, F.; Lai, F.; Hong, J.; Ding, X.; Yang, Z.; et al. Profiling of mismatch discrimination in RNAi enabled rational design of allele-specific siRNAs. Nucleic Acids Res 2009, 37, 7560-7569, doi: 10.1093/nar/gkp835.
19. Ohnishi, Y.; Tokunaga, K.; Hohjoh, H. Influence of assembly of siRNA elements into RNA-induced silencing complex by fork-siRNA duplex carrying nucleotide mismatches at the 3′- or 5′-end of the sense-stranded siRNA element. Biochem Biophys Res Commun 2005, 329, 516-521, doi: 10.1016/j.bbrc.2005.02.012.
20. Ivashkiv, L. B.; Donlin, L. T. Regulation of type I interferon responses. Nat Rev Immunol 2014, 14, 36-49, doi: 10.1038/nri3581.
21. Jin, L.; Han, Z.; Platisa, J.; Wooltorton, J. R.; Cohen, L. B.; Pieribone, V. A. Single action potentials and subthreshold electrical events imaged in neurons with a fluorescent protein voltage probe. Neuron 2012, 75, 779-785, doi: 10.1016/j.neuron.2012.06.040.
22. Mazzanti, A.; Maragna, R.; Vacanti, G.; Monteforte, N.; Bloise, R.; Marino, M.; Braghieri, L.; Gambelli, P.; Memmi, M.; Pagan, E.; et al. Interplay Between Genetic Substrate, QTc Duration, and Arrhythmia Risk in Patients With Long QT Syndrome. J Am Coll Cardiol 2018, 71, 1663-1671, doi: 10.1016/j.jacc.2018.01.078.
23. Vanoye, C. G.; Desai, R. R.; Fabre, K. L.; Gallagher, S. L.; Potet, F.; DeKeyser, J. M.; Macaya, D.; Meiler, J.; Sanders, C. R.; George, A. L., Jr. High-Throughput Functional Evaluation of KCNQ1 Decrypts Variants of Unknown Significance. Circ Genom Precis Med 2018, 11, e002345, doi: 10.1161/circgen.118.002345.
24. Jiang, J.; Wakimoto, H.; Seidman, J. G.; Seidman, C. E. Allele-specific silencing of mutant Myh6 transcripts in mice suppresses hypertrophic cardiomyopathy. Science 2013, 342, 111-114, doi: 10.1126/science. 1236921.
25. Trochet, D.; Prudhon, B.; Beuvin, M.; Peccate, C.; Lorain, S.; Julien, L.; Benkhelifa-Ziyyat, S.; Rabai, A.; Mamchaoui, K.; Ferry, A.; et al. Allele-specific silencing therapy for Dynamin 2-related dominant centronuclear myopathy. EMBO Mol Med 2018, 10, 239-253, doi: 10.15252/emmm.201707988.
26. Moss, A. J.; Shimizu, W.; Wilde, A. A.; Towbin, J. A.; Zareba, W.; Robinson, J. L.; Qi, M.; Vincent, G. M.; Ackerman, M. J.; Kaufman, E. S.; et al. Clinical aspects of type-1 long-QT syndrome by location, coding type, and biophysical function of mutations involving the KCNQ1 gene. Circulation 2007, 115, 2481-2489, doi: 10.1161/circulationaha.106.665406.
27. Ma, D.; Wei, H.; Lu, J.; Huang, D.; Liu, Z.; Loh, L. J.; Islam, O.; Liew, R.; Shim, W.; Cook, S. A. Characterization of a novel KCNQ1 mutation for type 1 long QT syndrome and assessment of the therapeutic potential of a novel IKs activator using patient-specific induced pluripotent stem cell-derived cardiomyocytes. Stem Cell Res Ther 2015, 6, 39, doi: 10.1186/s13287-015-0027-z.
28. Takaki, T.; Inagaki, A.; Chonabayashi, K.; Inoue, K.; Miki, K.; Ohno, S.; Makiyama, T.; Horie, M.; Yoshida, Y. Optical Recording of Action Potentials in Human Induced Pluripotent Stem Cell-Derived Cardiac Single Cells and Monolayers Generated from Long QT Syndrome Type 1 Patients. Stem Cells Int 2019, 2019, 7532657, doi: 10.1155/2019/7532657.
29. Sala, L.; Bellin, M.; Mummery, C. L. Integrating cardiomyocytes from human pluripotent stem cells in safety pharmacology: has the time come? Br J Pharmacol 2017, 174, 3749-3765, doi: 10.1111/bph.13577.
30. Verkerk, A. O.; Wilders, R. Dynamic Clamp in Electrophysiological Studies on Stem Cell-Derived Cardiomyocytes-Why and How? J Cardiovasc Pharmacol 2021, 77, 267-279, doi: 10.1097/fjc.0000000000000955.
31. Ma, J.; Guo, L.; Fiene, S.; Anson, B. D.; Thomson, J. A.; Kamp, T. J.; Kolaja, K. L.; Swanson, B. J.; January, C. T. High purity human-induced pluripotent stem cell-derived cardiomyocytes: electrophysiological properties of action potentials and ionic currents. Am J Physiol Heart Circ Physiol 2011, 301, H2006-2017, doi: 10.1152/ajpheart.00694.2011.
32. Vincent, G. M.; Timothy, K. W.; Leppert, M.; Keating, M. The spectrum of symptoms and QT intervals in carriers of the gene for the long-QT syndrome. N Engl J Med 1992, 327, 846-852, doi: 10.1056/nejm 199209173271204.
33. Harrison, S. M.; Riggs, E. R.; Maglott, D. R.; Lee, J. M.; Azzariti, D. R.; Niehaus, A.; Ramos, E. M.; Martin, C. L.; Landrum, M. J.; Rehm, H. L. Using ClinVar as a Resource to Support Variant Interpretation. Curr Protoc Hum Genet 2016, 89, 8.16.11-18.16.23, doi: 10.1002/0471142905.hg0816s89.
34. Wei, N.; Zhang, L.; Huang, H.; Chen, Y.; Zheng, J.; Zhou, X.; Yi, F.; Du, Q.; Liang, Z. siRNA has greatly elevated mismatch tolerance at 3′-UTR sites. PLOS One 2012, 7, e49309, doi: 10.1371/journal.pone.0049309.
35. Pfister, E. L.; Kennington, L.; Straubhaar, J.; Wagh, S.; Liu, W.; DiFiglia, M.; Landwehrmeyer, B.; Vonsattel, J. P.; Zamore, P. D.; Aronin, N. Five siRNAs targeting three SNPs may provide therapy for three-quarters of Huntington's disease patients. Curr Biol 2009, 19, 774-778, doi: 10.1016/j.cub.2009.03.030.
36. de Jong, A.; Dirven, R. J.; Boender, J.; Atiq, F.; Anvar, S. Y.; Leebeek, F. W. G.; van Vlijmen, B. J. M.; Eikenboom, J. Ex vivo Improvement of a von Willebrand Disease Type 2A Phenotype Using an Allele-Specific Small-Interfering RNA. Thromb Haemost 2020, 120, 1569-1579, doi: 10.1055/s-0040-1715442.
37. Boink, G. J.; Robinson, R. B. Gene therapy for restoring heart rhythm. J Cardiovasc Pharmacol Ther 2014, 19, 426-438, doi: 10.1177/1074248414528575.
38. Lorenzer, C.; Dirin, M.; Winkler, A. M.; Baumann, V.; Winkler, J. Going beyond the liver: progress and challenges of targeted delivery of siRNA therapeutics. J Control Release 2015, 203, 1-15, doi: 10.1016/j.jconrel.2015.02.003.
39. Kuzmin, D. A.; Shutova, M. V.; Johnston, N. R.; Smith, O. P.; Fedorin, V. V.; Kukushkin, Y. S.; van der Loo, J. C. M.; Johnstone, E. C. The clinical landscape for AAV gene therapies. Nat Rev Drug Discov 2021, 20, 173-174, doi: 10.1038/d41573-021-00017-7.
40. Boukens, B. J.; Walton, R.; Meijborg, V. M.; Coronel, R. Transmural electrophysiological heterogeneity, the T-wave and ventricular arrhythmias. Prog Biophys Mol Biol 2016, 122, 202-214, doi: 10.1016/j.pbiomolbio.2016.05.009.
41. Itzhaki, I.; Maizels, L.; Huber, I.; Gepstein, A.; Arbel, G.; Caspi, O.; Miller, L.; Belhassen, B.; Nof, E.; Glikson, M.; et al. Modeling of catecholaminergic polymorphic ventricular tachycardia with patient-specific human-induced pluripotent stem cells. J Am Coll Cardiol 2012, 60, 990-1000, doi: 10.1016/j.jacc.2012.02.066.
42. Burridge, P. W.; Matsa, E.; Shukla, P.; Lin, Z. C.; Churko, J. M.; Ebert, A. D.; Lan, F.; Diecke, S.; Huber, B.; Mordwinkin, N. M.; et al. Chemically defined generation of human cardiomyocytes. Nature Methods 2014, 11, 855-860, doi: 10.1038/nmeth.2999.
43. Tohyama, S.; Hattori, F.; Sano, M.; Hishiki, T.; Nagahata, Y.; Matsuura, T.; Hashimoto, H.; Suzuki, T.; Yamashita, H.; Satoh, Y.; et al. Distinct metabolic flow enables large-scale purification of mouse and human pluripotent stem cell-derived cardiomyocytes. Cell Stem Cell 2013, 12, 127-137, doi: 10.1016/j.stem.2012.09.013.
44. Tijsen, A. J.; Cócera Ortega, L.; Reckman, Y. J.; Zhang, X.; van der Made, I.; Aufiero, S.; Li, J.; Kamps, S. C.; van den Bout, A.; Devalla, H. D.; et al. Titin Circular RNAs Create a Back-Splice Motif Essential for SRSF10 Splicing. Circulation 2021, 143, 1502-1512, doi: 10.1161/circulationaha.120.050455.
45. Shinnawi, R.; Huber, I.; Maizels, L.; Shaheen, N.; Gepstein, A.; Arbel, G.; Tijsen, A. J.; Gepstein, L. Monitoring Human-Induced Pluripotent Stem Cell-Derived Cardiomyocytes with Genetically Encoded Calcium and Voltage Fluorescent Reporters. Stem Cell Reports 2015, 5, 582-596, doi: 10.1016/j.stemcr.2015.08.009.
46. Ruijter, J. M.; Ramakers, C.; Hoogaars, W. M.; Karlen, Y.; Bakker, O.; van den Hoff, M. J.; Moorman, A. F. Amplification efficiency: linking baseline and bias in the analysis of quantitative PCR data. Nucleic Acids Res 2009, 37, e45, doi: 10.1093/nar/gkp045.
47. ten Tusscher, K. H.; Noble, D.; Noble, P. J.; Panfilov, A. V. A model for human ventricular tissue. Am J Physiol Heart Circ Physiol 2004, 286, H1573-1589, doi: 10.1152/ajpheart.00794.2003.
48. ten Tusscher, K. H.; Panfilov, A. V. Alternans and spiral breakup in a human ventricular tissue model. Am J Physiol Heart Circ Physiol 2006, 291, H1088-1100, doi: 10.1152/ajpheart.00109.2006.
49. Ruijter, J. M.; Thygesen, H. H.; Schoneveld, O. J.; Das, A. T.; Berkhout, B.; Lamers, W. H. Factor correction as a tool to eliminate between-session variation in replicate experiments: application to molecular biology and retrovirology. Retrovirology 2006, 3, 2, doi: 10.1186/1742-4690-3-2.

### SEQUENCE LISTING

<110> Academisch Medisch Centrum
<120> ANTISENSE NUCLEIC ACIDS FOR USE IN THE TREATMENT FOR KCNQ1 MUTATION CARRIERS
<130> 2021-101
<160> 173
<170> BISSAP 1.3.6
<210> 1
<211> 37
<212> RNA
<213> Artificial Sequence
<220>
<223> Target sequence
<400> 1
cgucauugag caguacucgc agggccaccu caaccuc 37
<210> 2
<211> 37
<212> RNA
<213> Artificial Sequence
<220>
<223> Target sequence
<400> 2
cgucauugag caguacucac agggccaccu caaccuc 37
<210> 3
<211> 37
<212> RNA
<213> Artificial Sequence
<220>
<223> Target Sequence
<400> 3
gagguugagg uggcccugcg aguacugcuc aaugacg 37
<210> 4
<211> 37
<212> RNA
<213> Artificial Sequence
<220>
<223> Target sequence
<400> 4
gagguugagg uggcccugug aguacugcuc aaugacg 37
<210> 5
<211> 59
<212> DNA
<213> Artificial Sequence
<220>
<223> A18_fw
<400> 5
ccggaacaca gggccacctc aaccttcaag acaggttgag gtggccctgt gtttttttg 59
<210> 6
<211> 59
<212> DNA
<213> Artificial Sequence
<220>
<223> A18_rv
<400> 6
aattcaaaaa aacacagggc cacctcaacc tgtcttgaag gttgaggtgg ccctgtgtt 59
<210> 7
<211> 59
<212> DNA
<213> Artificial Sequence
<220>
<223> A10_fw
<400> 7
ccggaagcag tactcacagg gccactcaag acgtggccct gtgagtactg ctttttttg 59
<210> 8
<211> 59
<223> A10_rv
<400> 8
aattcaaaaa aagcagtact cacagggcca cgtcttgagt ggccctgtga gtactgctt 59
<210> 9
<211> 59
<212> DNA
<213> Artificial Sequence
<220>
<223> A13_fw
<400> 9
ccggaagtac tcacagggcc acctctcaag acgaggtggc cctgtgagta ctttttttg 59
<210> 10
<211> 59
<212> DNA
<213> Artificial Sequence
<220>
<223> A13_rv
<400> 10
aattcaaaaa aagtactcac agggccacct cgtcttgaga ggtggccctg tgagtactt 59
<210> 11
<211> 59
<212> DNA
<213> Artificial Sequence
<220>
<223> A15_fw
<400> 11
ccggaaactc acagggccac ctcaatcaag acttgaggtg gccctgtgag ttttttttg 59
<210> 12
<211> 5
<223> A15_rv
<400> 12
aattcaaaaa aaactcacag ggccacctca agtcttgatt gaggtggccc tgtgagttt 59
<210> 13
<211> 59
<212> DNA
<213> Artificial Sequence
<220>
<223> A16_fw
<400> 13
ccggaactca cagggccacc tcaactcaag acgttgaggt ggccctgtga gtttttttg 59
<210> 14
<211> 59
<212> DNA
<213> Artificial Sequence
<220>
<223> A16_rv
<400> 14
aattcaaaaa aactcacagg gccacctcaa cgtcttgagt tgaggtggcc ctgtgagtt 59
<210> 15
<211> 59
<212> DNA
<213> Artificial Sequence
<220>
<223> G18
<400> 15
ccggaacgca gggccacctc aaccttcaag acaggttgag gtggccctgc gtttttttg 59
<210> 16
<211> 59
<212> DNA
<213> Artificial Sequence
<220>
<223> G18_rv
<400> 16
aattcaaaaa aacgcagggc cacctcaacc tgtcttgaag gttgaggtgg ccctgcgtt 59
<210> 1
<211> 59
<212> DNA
<213> Artificial Sequence
<220>
<223> G10
<400> 17
ccggaagcag tactcgcagg gccactcaag acgtggccct gcgagtactg ctttttttg 59
<210> 18
<211> 59
<212> DNA
<213> Artificial Sequence
<220>
<223> G10_rv
<400> 18
aattcaaaaa aagcagtact cgcagggcca cgtcttgagt ggccctgcga gtactgctt 59
<210> 19
<211> 59
<212> DNA
<213> Artificial Sequence
<220>
<223> G11
<400> 19
ccggaacagt actcgcaggg ccacctcaag acggtggccc tgcgagtact gtttttttg 59
<210> 20
<211> 59
<212> DNA
<213> Artificial Sequence
<220>
<223> G11_rv
<400> 20
aattcaaaaa aacagtactc gcagggccac cgtcttgagg tggccctgcg agtactgtt 59
<210> 21
<211> 59
<212> DNA
<213> Artificial Sequence
<220>
<223> G12_fw
<400> 21
ccggaaagta ctcgcagggc caccttcaag acaggtggcc ctgcgagtac ttttttttg 59
<210> 22
<211> 59
<212> DNA
<213> Artificial Sequence
<220>
<223> G12_rv
<400> 22
aattcaaaaa aaagtactcg cagggccacc tgtcttgaag gtggccctgc gagtacttt 59
<210> 23
<211> 59
<212> DNA
<213> Artificial Sequence
<220>
<223> G16_fw
<400> 23
ccggaactcg cagggccacc tcaactcaag acgttgaggt ggccctgcga gtttttttg 59
<210> 24
<211> 59
<212> DNA
<213> Artificial Sequence
<220>
<223> G16_rv
<400> 24
aattcaaaaa aactcgcagg gccacctcaa cgtcttgagt tgaggtggcc ctgcgagtt 59
<210> 25
<211> 59
<212> DNA
<213> Artificial Sequence
<220>
<223> G11m5_fw
<400> 25
ccggaacagt actcgcaggg gcacctcaag acggtgcccc tgcgagtact gtttttttg 59
<210> 26
<211> 59
<212> DNA
<213> Artificial Sequence
<220>
<223> G11m5_rv
<400> 26
aattcaaaaa aacagtactc gcaggggcac cgtcttgagg tgcccctgcg agtactgtt 59
<210> 27
<211> 59
<212> DNA
<213> Artificial Sequence
<220>
<223> G11m6_fw
<400> 27
ccggaacagt actcgcaggc ccacctcaag acggtgggcc tgcgagtact gtttttttg 59
<210> 28
<211> 59
<212> DNA
<213> Artificial Sequence
<220>
<223> G11m6_rv
<400> 28
aattcaaaaa aacagtactc gcaggcccac cgtcttgagg tgggcctgcg agtactgtt 59
<210> 29
<211> 59
<212> DNA
<213> Artificial Sequence
<220>
<223> G11m7_fw
<400> 29
ccggaacagt actcgcagcg ccacctcaag acggtggcgc tgcgagtact gtttttttg 59
<210> 30
<211> 59
<212> DNA
<213> Artificial Sequence
<220>
<223> G11m7_rv
<400> 30
aattcaaaaa aacagtactc gcagcgccac cgtcttgagg tggcgctgcg agtactgtt 59
<210> 31
<211> 59
<212> DNA
<213> Artificial Sequence
<220>
<223> G11fork_fw
<400> 31
ccggaacagt actcgcaggg ccatatcaag acggtggccc tgcgagtact gtttttttg 59
<210> 32
<211> 59
<212> DNA
<213> Artificial Sequence
<220>
<223> G11fork_rv
<400> 32
aattcaaaaa aacagtactc gcagggccac cgtcttgata tggccctgcg agtactgtt 59
<210> 33
<211> 59
<213> Artificial Sequence
<220>
<223> G12m5_fw
<400> 33
ccggaaagta ctcgcagggc gaccttcaag acaggtcgcc ctgcgagtac ttttttttg 59
<210> 34
<211> 59
<212> DNA
<213> Artificial Sequence
<220>
<223> G12m5_rv
<400> 34
aattcaaaaa aaagtactcg cagggcgacc tgtcttgaag gtcgccctgc gagtacttt 59
<210> 35
<211> 59
<212> DNA
<213> Artificial Sequence
<220>
<223> G12m6_fw
<400> 35
ccggaaagta ctcgcagggg caccttcaag acaggtgccc ctgcgagtac ttttttttg 59
<210> 36
<211> 59
<212> DNA
<213> Artificial Sequence
<220>
<223> G12m6_rv
<400> 36
aattcaaaaa aaagtactcg caggggcacc tgtcttgaag gtgcccctgc gagtacttt 59
<210> 37
<211> 59
<223> G12m7_fw
<400> 37
ccggaaagta ctcgcaggcc caccttcaag acaggtgggc ctgcgagtac ttttttttg 59
<210> 38
<211> 59
<212> DNA
<213> Artificial Sequence
<220>
<223> G12m7_rv
<400> 38
aattcaaaaa aaagtactcg caggcccacc tgtcttgaag gtgggcctgc gagtacttt 59
<210> 39
<211> 59
<212> DNA
<213> Artificial Sequence
<220>
<223> G12fork_fw
<400> 39
ccggaaagta ctcgcagggc cactatcaag acaggtggcc ctgcgagtac ttttttttg 59
<210> 40
<211> 59
<212> DNA
<213> Artificial Sequence
<220>
<223> G12fork_rv
<400> 40
aattcaaaaa aaagtactcg cagggccacc tgtcttgata gtggccctgc gagtacttt 59
<210> 41
<211> 59
<212> DNA
<213> Artificial Sequence
<220>
<223> G18m10_fw
<400> 41
ccggaacgca gggcctcctc aaccttcaag acaggttgag gaggccctgc gtttttttg 59
<210> 42
<211> 59
<212> DNA
<213> Artificial Sequence
<220>
<223> G18m10_rv
<400> 42
aattcaaaaa aacgcagggc ctcctcaacc tgtcttgaag gttgaggagg ccctgcgtt 59
<210> 43
<211> 59
<212> DNA
<213> Artificial Sequence
<220>
<223> G18m11
<400> 43
ccggaacgca gggcgacctc aaccttcaag acaggttgag gtcgccctgc gtttttttg 59
<210> 44
<211> 59
<212> DNA
<213> Artificial Sequence
<220>
<223> G18m11_rv
<400> 44
aattcaaaaa aacgcagggc gacctcaacc tgtcttgaag gttgaggtcg ccctgcgtt 59
<210> 45
<211> 59
<212> DNA
<213> Artificial Sequence
<220>
<223> G18fork_fw
<400> 45
ccggaacgca gggccacctc aactatcaag acaggttgag gtggccctgc gtttttttg 59
<210> 46
<211> 59
<212> DNA
<213> Artificial Sequence
<220>
<223> G18fork_rv
<400> 46
aattcaaaaa aacgcagggc cacctcaacc tgtcttgata gttgaggtgg ccctgcgtt 59
<210> 47
<211> 59
<212> DNA
<213> Artificial Sequence
<220>
<223> A18m11_fw
<400> 47
ccggaacaca gggcgacctc aaccttcaag acaggttgag gtcgccctgt gtttttttg 59
<210> 48
<211> 59
<212> DNA
<213> Artificial Sequence
<220>
<223> A18m11 rv
<400> 48
aattcaaaaa aacacagggc gacctcaacc tgtcttgaag gttgaggtcg ccctgtgtt 59
<210> 49
<211> 59
<212> DNA
<213> Artificial Sequence
<220>
<223> A18m10_fw
<400> 49
ccggaacaca gggcctcctc aaccttcaag acaggttgag gaggccctgt gtttttttg 59
<210> 50
<211> 59
<212> DNA
<213> Artificial Sequence
<220>
<223> A18m10_rv
<400> 50
aattcaaaaa aacacagggc ctcctcaacc tgtcttgaag gttgaggagg ccctgtgtt 59
<210> 51
<211> 59
<212> DNA
<213> Artificial Sequence
<220>
<223> A18fork_fw
<400> 51
ccggaacaca gggccacctc aactatcaag acaggttgag gtggccctgt gtttttttg 59
<210> 52
<211> 59
<212> DNA
<213> Artificial Sequence
<220>
<223> A18fork_rv
<400> 52
aattcaaaaa aacacagggc cacctcaacc tgtcttgata gttgaggtgg ccctgtgtt 59
<210> 53
<211> 59
<212> DNA
<213> Artificial Sequence
<220>
<223> rs8234_A8_fw
<400> 53
ccggaactgg gcattacatc gcatatcaag actatgcgat gtaatgccca gtttttttg 59
<210> 54
<211> 59
<212> DNA
<213> Artificial Sequence
<220>
<223> rs8234_A8_rv
<400> 54
aattcaaaaa aactgggcat tacatcgcat agtcttgata tgcgatgtaa tgcccagtt 59
<210> 55
<211> 59
<212> DNA
<213> Artificial Sequence
<220>
<223> rs8234_A10_fw
<400> 55
ccggaagggc attacatcgc atagatcaag actctatgcg atgtaatgcc ctttttttg 59
<210> 56
<211> 59
<212> DNA
<213> Artificial Sequence
<220>
<223> rs8234_A10_rv
<400> 56
aattcaaaaa aagggcatta catcgcatag agtcttgatc tatgcgatgt aatgccctt 59
<210> 57
<211> 59
<212> DNA
<213> Artificial Sequence
<220>
<223> rs8234_A11_fw
<400> 57
ccggaaggca ttacatcgca tagaatcaag acttctatgc gatgtaatgc ctttttttg 59
<210> 58
<211> 59
<212> DNA
<213> Artificial Sequence
<220>
<223> rs8234_A11_rv
<400> 58
aattcaaaaa aaggcattac atcgcataga agtcttgatt ctatgcgatg taatgcctt 59
<210> 59
<211> 59
<212> DNA
<213> Artificial Sequence
<220>
<223> rs8234_A12_fw
<400> 59
ccggaagcat tacatcgcat agaaatcaag actttctatg cgatgtaatg ctttttttg 59
<210> 60
<211> 59
<212> DNA
<213> Artificial Sequence
<220>
<223> rs8234_A12_rv
<400> 60
aattcaaaaa aagcattaca tcgcatagaa agtcttgatt tctatgcgat gtaatgctt 59
<210> 61
<211> 59
<212> DNA
<213> Artificial Sequence
<220>
<223> rs8234_A13_fw
<400> 61
ccggaacatt acatcgcata gaaattcaag acatttctat gcgatgtaat gtttttttg 59
<210> 62
<211> 59
<223> rs8234_A13_rv
<400> 62
aattcaaaaa aacattacat cgcatagaaa tgtcttgaat ttctatgcga tgtaatgtt 59
<210> 63
<211> 59
<212> DNA
<213> Artificial Sequence
<220>
<223> rs8234_A15_fw
<400> 63
ccggaattac atcgcataga aatcatcaag actgatttct atgcgatgta atttttttg 59
<210> 64
<211> 59
<212> DNA
<213> Artificial Sequence
<220>
<223> rs8234_A15_rv
<400> 64
aattcaaaaa aattacatcg catagaaatc agtcttgatg atttctatgc gatgtaatt 59
<210> 65
<211> 59
<212> DNA
<213> Artificial Sequence
<220>
<223> rs8234_A16_fw
<400> 65
ccggaataca tcgcatagaa atcaatcaag acttgatttc tatgcgatgt atttttttg 59
<210> 66
<211> 59
<223> rs8234_A16_rv
<400> 66
aattcaaaaa aatacatcgc atagaaatca agtcttgatt gatttctatg cgatgtatt 59
<210> 67
<211> 59
<212> DNA
<213> Artificial Sequence
<220>
<223> rs8234_A18_fw
<400> 67
ccggaacatc gcatagaaat caatatcaag actattgatt tctatgcgat gtttttttg 59
<210> 68
<211> 59
<212> DNA
<213> Artificial Sequence
<220>
<223> rs8234_A18_rv
<400> 68
aattcaaaaa aacatcgcat agaaatcaat agtcttgata ttgatttcta tgcgatgtt 59
<210> 69
<211> 59
<212> DNA
<213> Artificial Sequence
<220>
<223> rs8234_G8_fw
<400> 69
ccggaactgg gcattacgtc gcatatcaag actatgcgac gtaatgccca gtttttttg 59
<210> 70
<211> 59
<212> DNA
<213> Artificial Sequence
<220>
<223> rs8234_G8_rv
<400> 70
aattcaaaaa aactgggcat tacgtcgcat agtcttgata tgcgacgtaa tgcccagtt 59
<210> 71
<211> 59
<212> DNA
<213> Artificial Sequence
<220>
<223> rs8234_G10_fw
<400> 71
ccggaagggc attacgtcgc atagatcaag actctatgcg acgtaatgcc ctttttttg 59
<210> 72
<211> 59
<212> DNA
<213> Artificial Sequence
<220>
<223> rs8234_G10_rv
<400> 72
aattcaaaaa aagggcatta cgtcgcatag agtcttgatc tatgcgacgt aatgccctt 59
<210> 73
<211> 59
<212> DNA
<213> Artificial Sequence
<220>
<223> rs8234_G11_fw
<400> 73
ccggaaggca ttacgtcgca tagaatcaag acttctatgc gacgtaatgc ctttttttg 59
<210> 74
<211> 59
<212> DNA
<213> Artificial Sequence
<220>
<223> rs8234_G11_rv
<400> 74
aattcaaaaa aaggcattac gtcgcataga agtcttgatt ctatgcgacg taatgcctt 59
<210> 75
<211> 59
<212> DNA
<213> Artificial Sequence
<220>
<223> rs8234_G12_fw
<400> 75
ccggaagcat tacgtcgcat agaaatcaag actttctatg cgacgtaatg ctttttttg 59
<210> 76
<211> 59
<212> DNA
<213> Artificial Sequence
<220>
<223> rs8234_G12_rv
<400> 76
aattcaaaaa aagcattacg tcgcatagaa agtcttgatt tctatgcgac gtaatgctt 59
<210> 77
<211> 59
<212> DNA
<213> Artificial Sequence
<220>
<223> rs8234_G13_fw
<400> 77
ccggaacatt acgtcgcata gaaattcaag acatttctat gcgacgtaat gtttttttg 59
<210> 78
<211> 59
<212> DNA
<213> Artificial Sequence
<220>
<223> rs8234_G13_rv
<400> 78
aattcaaaaa aacattacgt cgcatagaaa tgtcttgaat ttctatgcga cgtaatgtt 59
<210> 79
<211> 59
<212> DNA
<213> Artificial Sequence
<220>
<223> rs8234_G16_fw
<400> 79
ccggaatacg tcgcatagaa atcaatcaag acttgatttc tatgcgacgt atttttttg 59
<210> 80
<211> 59
<212> DNA
<213> Artificial Sequence
<220>
<223> rs8234_G16_rv
<400> 80
aattcaaaaa aatacgtcgc atagaaatca agtcttgatt gatttctatg cgacgtatt 59
<210> 81
<211> 59
<212> DNA
<213> Artificial Sequence
<220>
<223> rs8234_G18_fw
<400> 81
ccggaacgtc gcatagaaat caatatcaag actattgatt tctatgcgac gtttttttg 59
<210> 82
<211> 59
<212> DNA
<213> Artificial Sequence
<220>
<223> rs8234_G18_rv
<400> 82
aattcaaaaa aacgtcgcat agaaatcaat agtcttgata ttgatttcta tgcgacgtt 59
<210> 83
<211> 19
<212> DNA
<213> Artificial Sequence
<220>
<223> G1 antisense
<400> 83
cgagtactgc tcaatgacg 19
<210> 84
<211> 1
<212> DNA
<213> Artificial Sequence
<220>
<223> G2 antisense
<400> 84
gcgagtactg ctcaatgac 19
<210> 85
<211> 19
<212> DNA
<213> Artificial Sequence
<220>
<223> G3 antisense
<400> 85
tgcgagtact gctcaatga 19
<210> 86
<211> 19
<212> DNA
<213> Artificial Sequence
<220>
<223> G4 antisense
<400> 86
ctgcgagtac tgctcaatg 19
<210> 87
<211> 19
<213> Artificial Sequence
<220>
<223> G5 antisense
<400> 87
cctgcgagta ctgctcaat 19
<210> 88
<211> 19
<212> DNA
<213> Artificial Sequence
<220>
<223> G6 antisense
<400> 88
ccctgcgagt actgctcaa 19
<210> 89
<211> 19
<212> DNA
<213> Artificial Sequence
<220>
<223> G7 antisense
<400> 89
gccctgcgag tactgctca 19
<210> 90
<211> 19
<212> DNA
<213> Artificial Sequence
<220>
<223> G8 antisense
<400> 90
ggccctgcga gtactgctc 19
<210> 91
<211> 19
<212> DNA
<223> G9 antisense
<400> 91
tggccctgcg agtactgct 19
<210> 92
<211> 19
<212> DNA
<213> Artificial Sequence
<220>
<223> G10 antisense
<400> 92
gtggccctgc gagtactgc 19
<210> 93
<211> 19
<212> DNA
<213> Artificial Sequence
<220>
<223> G11 antisense
<400> 93
ggtggccctg cgagtactg 19
<210> 94
<211> 19
<212> DNA
<213> Artificial Sequence
<220>
<223> G12 antisense
<400> 94
aggtggccct gcgagtact 19
<210> 95
<211> 19
<212> DNA
<213> Artificial Sequence
<220>
<223> G13 antisense
<400> 95
gaggtggccc tgcgagtac 19
<210> 96
<211> 19
<212> DNA
<213> Artificial Sequence
<220>
<223> G14 antisense
<400> 96
tgaggtggcc ctgcgagta 19
<210> 97
<211> 19
<212> DNA
<213> Artificial Sequence
<220>
<223> G15 antisense
<400> 97
ttgaggtggc cctgcgagt 19
<210> 98
<211> 19
<212> DNA
<213> Artificial Sequence
<220>
<223> G16 antisense
<400> 98
gttgaggtgg ccctgcgag 19
<210> 99
<211> 19
<212> DNA
<213> Artificial Sequence
<220>
<223> G17 antisense
<400> 99
ggttgaggtg gccctgcga 19
<210> 100
<211> 19
<212> DNA
<213> Artificial Sequence
<220>
<223> G18 antisense
<400> 100
aggttgaggt ggccctgcg 19
<210> 101
<211> 19
<212> DNA
<213> Artificial Sequence
<220>
<223> G19 antisense
<400> 101
gaggttgagg tggccctgc 19
<210> 102
<211> 19
<212> DNA
<213> Artificial Sequence
<220>
<223> A1 antisense
<400> 102
tgagtactgc tcaatgacg 19
<210> 103
<211> 19
<212> DNA
<213> Artificial Sequence
<220>
<223> A2 antisense
<400> 103
gtgagtactg ctcaatgac 19
<210> 104
<211> 19
<212> DNA
<213> Artificial Sequence
<220>
<223> A3 antisense
<400> 104
tgtgagtact gctcaatga 19
<210> 105
<211> 19
<212> DNA
<213> Artificial Sequence
<220>
<223> A4 antisense
<400> 105
ctgtgagtac tgctcaatg 19
<210> 106
<211> 19
<212> DNA
<213> Artificial Sequence
<220>
<223> A5 antisense
<400> 106
cctgtgagta ctgctcaat 19
<210> 107
<211> 19
<212> DNA
<213> Artificial Sequence
<220>
<223> A6 antisense
<400> 107
ccctgtgagt actgctcaa 19
<210> 108
<211> 19
<212> DNA
<213> Artificial Sequence
<220>
<223> A7 antisense
<400> 108
gccctgtgag tactgctca 19
<210> 109
<211> 19
<212> DNA
<213> Artificial Sequence
<220>
<223> A8 antisense
<400> 109
ggccctgtga gtactgctc 19
<210> 110
<211> 19
<212> DNA
<213> Artificial Sequence
<220>
<223> A9 antisense
<400> 110
tggccctgtg agtactgct 19
<210> 111
<211> 19
<212> DNA
<213> Artificial Sequence
<220>
<223> A10 antisense
<400> 111
gtggccctgt gagtactgc 19
<210> 112
<211> 19
<212> DNA
<213> Artificial Sequence
<220>
<223> A11 antisense
<400> 112
ggtggccctg tgagtactg 19
<210> 113
<211> 19
<212> DNA
<213> Artificial Sequence
<220>
<223> A12 antisense
<400> 113
aggtggccct gtgagtact 19
<210> 114
<211> 19
<212> DNA
<213> Artificial Sequence
<220>
<223> A13 antisense
<400> 114
gaggtggccc tgtgagtac 19
<210> 115
<211> 19
<212> DNA
<213> Artificial Sequence
<220>
<223> A14 antisense
<400> 115
tgaggtggcc ctgtgagta 19
<210> 116
<211> 19
<223> A15 antisense
<400> 116
ttgaggtggc cctgtgagt 19
<210> 117
<211> 19
<212> DNA
<213> Artificial Sequence
<220>
<223> A16 antisense
<400> 117
gttgaggtgg ccctgtgag 19
<210> 118
<211> 19
<212> DNA
<213> Artificial Sequence
<220>
<223> A17 antisense
<400> 118
ggttgaggtg gccctgtga 19
<210> 119
<211> 19
<212> DNA
<213> Artificial Sequence
<220>
<223> A18 antisense
<400> 119
aggttgaggt ggccctgtg 19
<210> 120
<211> 19
<223> A19 antisense
<400> 120
gaggttgagg tggccctgt 19
<210> 121
<211> 21
<212> DNA
<213> Artificial Sequence
<220>
<223> KCNQ1_wt fw
<400> 121
tgaggatgct acacgtcgac c 21
<210> 122
<211> 23
<212> DNA
<213> Artificial Sequence
<220>
<223> KCNQ1_wt rev
<400> 122
agccgatgta cagggtggtt atc 23
<210> 123
<211> 21
<212> DNA
<213> Artificial Sequence
<220>
<223> KCNQ1_mut fw
<400> 123
tgaggatgct acacgtcgac t 21
<210> 124
<211> 2
<212> DNA
<213> Artificial Sequence
<220>
<223> KCNQ1_mut rev
<400> 124
agccgatgta cagggtggtt atc 23
<210> 125
<211> 20
<212> DNA
<213> Artificial Sequence
<220>
<223> KCNQ1_total fw
<400> 125
tcctgaggat gctacacgtc 20
<210> 126
<211> 23
<212> DNA
<213> Artificial Sequence
<220>
<223> KCNQ1_total rev
<400> 126
agccgatgta cagggtggtt atc 23
<210> 127
<211> 22
<212> DNA
<213> Artificial Sequence
<220>
<223> STAT1 fw
<400> 127
atcacattca catgggtgga gc 22
<210> 128
<211> 25
<212> DNA
<213> Artificial Sequence
<220>
<223> STAT1 rev
<400> 128
acagatactt caggggattc tcagg 25
<210> 129
<211> 20
<212> DNA
<213> Artificial Sequence
<220>
<223> OAS1 fw
<400> 129
catccgccta gtcaagcact 20
<210> 130
<211> 21
<212> DNA
<213> Artificial Sequence
<220>
<223> OAS1 rev
<400> 130
aagaccgtcc gaaatccctg g 21
<210> 131
<211> 21
<212> DNA
<213> Artificial Sequence
<220>
<223> GAPDH fw
<400> 131
acccactoct ccacctttga c 21
<210> 132
<211> 22
<212> DNA
<213> Artificial Sequence
<220>
<223> GAPDH rev
<400> 132
accctgttgc tgtagccaaa tt 22
<210> 133
<211> 21
<212> DNA
<213> Artificial Sequence
<220>
<223> HPRT fw
<400> 133
tgacactggc aaaacaatgc a 21
<210> 134
<211> 2
<212> DNA
<213> Artificial Sequence
<220>
<223> HPRT rev
<400> 134
ggtccttttc accagcaagc t 21
<210> 135
<211> 21
<212> DNA
<213> Artificial Sequence
<220>
<223> TBP fw
<400> 135
gctcacccac caacaattta g 21
<210> 136
<211> 22
<212> DNA
<213> Artificial Sequence
<220>
<223> TBP rev
<400> 136
tctgctctga ctttagcacc tg 22
<210> 137
<211> 23
<212> DNA
<213> Artificial Sequence
<220>
<223> NANOG fw
<400> 137
agaatagcaa tggtgtgacg cag 23
<210> 138
<211> 2
<212> DNA
<213> Artificial Sequence
<220>
<223> NANOG rev
<400> 138
tggatgttct gggtctggtt gc 22
<210> 139
<211> 20
<212> DNA
<213> Artificial Sequence
<220>
<223> OCT4 fw
<400> 139
tggttggagg gaaggtgaag 20
<210> 140
<211> 21
<212> DNA
<213> Artificial Sequence
<220>
<223> OCT4 rev
<400> 140
tgtctatcta ctgtgtccca g 21
<210> 141
<211> 20
<213> Artificial Sequence
<220>
<223> OCT4_viral fw
<400> 141
tgtactoctc ggtccctttc 20
<210> 142
<211> 19
<212> DNA
<213> Artificial Sequence
<220>
<223> OCT4_viral rev
<400> 142
caggtggggt ctttcattc 19
<210> 143
<211> 21
<212> DNA
<213> Artificial Sequence
<220>
<223> SOX2 fw
<400> 143
accaatccca tccacactca c 21
<210> 144
<211> 22
<212> DNA
<213> Artificial Sequence
<220>
<223> SOX2 rev
<400> 144
tctatacaag gtccattccc cc 22
<210> 145
<211> 19
<212> DNA
<223> SOX2_viral fw
<400> 145
atcccagtgt ggtggtacg 19
<210> 146
<211> 21
<212> DNA
<213> Artificial Sequence
<220>
<223> SOX2_viral rev
<400> 146
aaggcattca tgggccgctt g 21
<210> 147
<211> 21
<212> DNA
<213> Artificial Sequence
<220>
<223> MLC2V fw
<400> 147
acaactgaca ccaacacctg c 21
<210> 148
<211> 22
<212> DNA
<213> Artificial Sequence
<220>
<223> MLC2V rev
<400> 148
agtccaagtt gccagtcacg tc 22
<210> 149
<211> 20
<212> DNA
<213> Artificial Sequence
<220>
<223> NKX2.5 fw
<400> 149
tctatccacg tgcctacagc 20
<210> 150
<211> 21
<212> DNA
<213> Artificial Sequence
<220>
<223> NKX2.5 rev
<400> 1!
agaaagtcag gctggctcaa g 21
<210> 151
<211> 21
<212> DNA
<213> Artificial Sequence
<220>
<223> KLF4_viral fw
<400> 151
ctgcggcaaa acctacacaa a 21
<210> 152
<211> 22
<212> DNA
<213> Artificial Sequence
<220>
<223> KLF4_viral rev
<400> 152
ttatcgtcga ccactgtgct gg 22
<210> 153
<211> 21
<212> DNA
<213> Artificial Sequence
<220>
<223> MYH6 fw
<400> 153
acctgtccaa gttccgcaag g 21
<210> 154
<211> 22
<212> DNA
<213> Artificial Sequence
<220>
<223> MYH6 rev
<400> 154
ttacaggttg gcaagagtga gg 22
<210> 155
<211> 21
<212> DNA
<213> Artificial Sequence
<220>
<223> MYH7 fw
<400> 155
acctgtccaa gttccgcaag g 21
<210> 156
<211> 19
<212> DNA
<213> Artificial Sequence
<220>
<223> MYH7 rev
<400> 156
tttgctggca cctccaggg 19
<210> 157
<211> 35
<212> DNA
<213> Artificial Sequence
<220>
<223> Fig 1 p. 1 Target
<400> 157
gtcattgagc agtactcaca gggccacctc aacct 35
<210>
<211> 35
<212> DNA
<213> Artificial Sequence
<220>
<223> Fig 1 p. 2 Target
<400> 158
gtcattgagc agtactcgca gggccacctc aacct 35
<210> 159
<211> 19
<212> DNA
<213> Artificial Sequence
<220>
<223> A18m11
<400> 159
aggttgaggt cgccctgtg 19
<210> 160
<211> 19
<212> DNA
<213> Artificial Sequence
<220>
<223> A18m10
<400> 160
aggttgagga ggccctgtg 19
<210> 161
<211> 19
<212> DNA
<213> Artificial Sequence
<220>
<223> A18fork
<400> 161
aggttgaggt ggccctgtg 19
<210> 162
<211> 19
<212> DNA
<213> Artificial Sequence
<220>
<223> G11m5
<400> 162
ggtgcccctg cgagtactg 19
<210> 163
<211> 19
<212> DNA
<213> Artificial Sequence
<220>
<223> G11m6
<400> 163
ggtgcgcctg cgagtactg 19
<210> 164
<211> 19
<212> DNA
<213> Artificial Sequence
<220>
<223> G11m7
<400> 164
gtcatgagcg tcgcggtgg 19
<210> 165
<211> 19
<212> DNA
<213> Artificial Sequence
<220>
<223> G11mfork
<400> 165
ggtggccctg cgagtactg 19
<210> 166
<211> 19
<212> DNA
<213> Artificial Sequence
<220>
<223> G12m5
<400> 166
aggtcgccct gcgagtact 19
<210> 167
<211> 19
<212> DNA
<213> Artificial Sequence
<220>
<223> G12m6
<400> 167
aggtgcccct gcgagtact 19
<210> 168
<211> 19
<212> DNA
<213> Artificial Sequence
<220>
<223> G12m7
<400> 168
aggtgggcct gcgagtact 19
<210> 169
<211> 19
<212> DNA
<213> Artificial Sequence
<220>
<223> G12mfork
<400> 169
aggtggccct gcgagtact 19
<210> 170
<211> 19
<223> G18m10
<400> 170
aggttgagga ggccctgcg 19
<210> 171
<211> 19
<212> DNA
<213> Artificial Sequence
<220>
<223> G18m11
<400> 171
aggttgaggt cgccctgcg 19
<210> 172
<211> 19
<212> DNA
<213> Artificial Sequence
<220>
<223> G18fork
<400> 172
aggttgaggt ggccctgcg 19
<210> 173
<211> 12
<212> DNA
<213> Artificial Sequence
<220>
<223> p. 3 and p. 29
<400> 173
aattcaaaaa aa 12

1. An isolated antisense molecule capable of inhibiting the expression of an allele of a KCNQ1 gene in a mammalian cell, wherein said antisense molecule comprises an antisense nucleic acid strand which is substantially complementary to a target region of a transcript encoded by the KCNQ1 gene, wherein said antisense nucleic acid strand is at least complementary to SNP rs1057128, rs8234 or rs17215465 in said target region.

2. The isolated antisense molecule of claim 1 selected from:
a. a double stranded nucleic acid (dsNA) or a chemically modified version thereof, or
b. an antisense oligonucleotide (ASO)

3. The isolated antisense molecule according to claim 1, wherein said target region comprises the nucleic acid sequence a. CGUCAUUGAGCAGUACUCGCAGGGCCACCUCAACCUC (SEQ ID NO:1), or b. CGUCAUUGAGCAGUACUCACAGGGCCACCUCAACCUC (SEQ ID NO:2), or a sequence being at least 80% homologous thereto.

4. The isolated antisense molecule according to claim 1, wherein said antisense nucleic acid strand comprises a nucleic acid sequence having a consecutive strand of at least 12 nucleotides selected from SEQ ID NO:3 or SEQ ID NO:4 or a nucleic acid analogue sequence thereof.

5. The isolated antisense molecule of claim 1, comprising the antisense nucleic acid selected from one of SEQ ID NO: 83-120 or a nucleic acid analogue sequence thereof.

6. The isolated antisense molecule of claim 1, comprising the antisense nucleic acid according to SEQ ID NO: 93 or 119 or a nucleic acid analogue sequence thereof.

7. The isolated antisense molecule according to claim 1, wherein said antisense molecule comprises a sense nucleic acid strand of 15-30 nucleotides in length; and wherein the sense nucleic acid strand is complementary to said antisense region and wherein the sense and antisense nucleic acid strands form a duplex region.

8. The isolated antisense molecule according to claim 1, wherein the antisense strand and the sense strand are operably linked by means of an RNA loop strand to form a hairpin structure comprising a duplex structure and a loop structure.

9. A nucleic acid encoding the isolated antisense molecule of claim 1.

10. An expression cassette comprising a nucleic acid encoding said antisense molecule of claim 1.

11. An expression vector comprising the expression cassette of claim 10.

12. A pharmaceutical composition comprising the isolated antisense molecule of claim 1, and a pharmaceutically acceptable carrier.

13. A method of treating an individual in need thereof comprising administering to the individual the isolated antisense molecule according to claim 1.

14. A method of treating an individual for cardiac arrhythmia comprising administering to the individual the isolated antisense molecule according to claim 1.

15. An in vitro method for inhibiting the expression of an allele of a KCNQ1 gene in a cell, comprising the following steps:
a. introducing into the cell expressing a KCNQ1 mutant an antisense molecule of claim 1 which inhibits expression of the KCNQ1 mutant allele; and
b. maintaining the cell produced in step (a) for a time sufficient to obtain degradation of the mRNA transcript of the KCNQ1 mutant allele, thereby inhibiting expression of the KCNQ1 mutant allele in the cell.

16. A pharmaceutical composition comprising the expression cassette of claim 10, and a pharmaceutically acceptable carrier.

17. A pharmaceutical composition comprising the expression vector of claim 11, and a pharmaceutically acceptable carrier.

18. A method of treating an individual in need thereof comprising administering to the individual the expression cassette of claim 10.

19. A method of treating an individual in need thereof comprising administering to the individual the expression vector of claim 11.

20. A method of treating an individual in need thereof comprising administering to the individual the pharmaceutical composition of claim 16.

21. A method of treating an individual in need thereof comprising administering to the individual the pharmaceutical composition of claim 17.
